(19) United States
(12) Patent Application Publication
(10) Pub. No.: US 2024/0199535 A1
(43) Pub. Date: Jun. 20, 2024

(54) DEUTERATED DHODH INHIBITORS

(71) Applicant: IMMUNIC AG, Gräfelfing (DE)

(72) Inventors: Christian GEGE, Ehingen (DE); Hella KOHLHOF, München (DE); Andreas MÜHLER, München (DE); Daniel VITT, Germering (DE)

(73) Assignee: IMMUNIC AG, Gräfelfing (DE)

(21) Appl. No.: 18/554,365

(22) PCT Filed: Apr. 8, 2022
(86) PCT No.: PCT/EP2022/059527

§ 371 Date: Oct. 6, 2023

(30) Foreign Application Priority Data
Apr. 9, 2021 (EP) 21167690.3
Jun. 23, 2021 (EP) 21181134.4

(51) Int. Cl. C07C 233/75; A61K 31/16; A61K 31/167; A61K 31/341; A61K 31/381; A61K 31/426; A61K 31/437; A61K 31/44; A61K 31/472; A61K 31/5375; A61P 31/14; C07C 233/74; C07D 213/68; C07D 213/73; C07D 217/04; C07D 265/30; C07D 277/34; C07D 307/30; C07D 333/40; C07D 471/04

(52) U.S. Cl. CPC C07C 233/75; A61K 31/16; A61K 31/167; A61K 31/341; A61K 31/381; A61K 31/426; A61K 31/437; A61K 31/44; A61K 31/472; A61K 31/5375; A61P 31/14; C07C 233/74; C07D 213/68; C07D 213/73; C07D 217/04; C07D 265/30; C07D 277/34; C07D 307/30; C07D 333/40; C07D 471/04

(57) ABSTRACT

The invention relates to novel deuterated compounds of Formula (I)

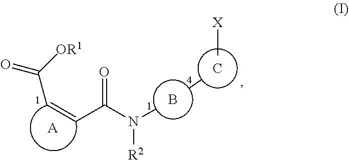

and their use as medicaments.

### SUMMARY OF THE INVENTION

The present disclosure relates to novel deuterated dihydroorotate dehydrogenase (DHODH) inhibitors, pharmaceutical formulations comprising them, a process for their preparation and their use as medicament, alone or in combination with one or more additional agents, for treating of various diseases, wherein the inhibition of DHODH is desirable.

### BACKGROUND OF THE INVENTION

Vidofludimus calcium (IMU-838) is a selective and potent second-generation dihydroorotate dehydrogenase (DHODH) oral immunomodulator being developed for the treatment of several chronic inflammatory diseases, including relapsing-remitting Multiple Sclerosis (rrMS):

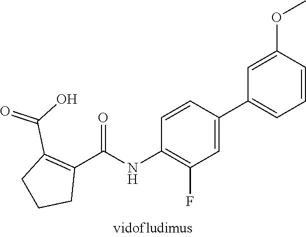 [SMILES: COc1cccc(-c2ccc(NC(=O)C3=C(C(=O)O)CCC3)c(F)c2)c1]

The mechanism of action of vidofludimus calcium, a small molecule selective immune modulator, is the inhibition of the intracellular metabolism of activated immune T- and B-cells by blocking the enzyme DHODH. The inhibition of the DHODH enzyme leads to metabolic stress in metabolically activated lymphocytes resulting in reduction in proinflammatory cytokines and subsequently to apoptosis of activated immune cells. Blocking of the DHODH enzyme activity has a selective effect to metabolically activated immune cells, to malignant cells and to virus-infected cells. Thus, DHODH inhibition should therefore not lead to general antiproliferative effects in other cells. IMU-838 as a second-generation DHODH inhibitor is being developed to separate the desired immunomodulatory effects from an undesirable side effect profile caused by off-target effects like neutropenia, alopecia and diarrhea. An additional benefit of DHODH inhibitors such as IMU-838 is their direct antiviral effect. During long-term treatment with immunosuppressive drugs, the reactivation of latent viruses has been observed. This can lead to serious infections, such as progressive multifocal leukoencephalopathy which can have a lethal outcome.

PP-001 is another DHODH inhibitor within the same structural class for the treatment of retinal diseases like uveitis, diabetic macular edema and retinal vein occlusion currently in clinical trials. In animal models the high effectiveness to treat dry eye disease and viral conjunctivitis has already been demonstrated.

There is a need to develop novel DHODH inhibitors. In particular, there is a need to develop DHODH inhibitors with improved pharmacokinetic properties. The covalent C—H bond is weaker than an otherwise identical C-D bond due to the kinetic isotope effect. The breaking of C—H bonds is a common feature of drug metabolism and breaking of an analogous C-D bond can be more difficult and so decreases the rate of metabolism. Replacement of H with D in small molecules can lead to significant reductions in metabolism leading to beneficial changes in the biological effects of drugs. Replacement may also have the effect of lowering toxicity by reducing the formation of a toxic metabolite (J. Med. Chem. 2019; 62:5276). Deuterated analogs share the beneficial mechanism of action, however are expected to be metabolized slower and with less variability between patients compared with the non-deuterated matched pair. It is generally believed that a differentiated pharmacokinetic profile could enable potentially improved efficacy, less frequent dosing, improved tolerability, reduced interpatient variability in drug metabolism and reduced drug-drug interactions.

### PRIOR ART

Non-deuterated compounds of Formula (I) are described in WO2004/056746, WO2004/056747, WO2004/056797, WO2010/052027, WO2010/128050, WO2012/001148, WO2012/001151, WO2015/169944, WO2015/154820, WO2019/170848, WO2019/101888, WO2019/175396 as well as in Bioorg. Med. Chem. Lett. 2004; 14:55, Bioorg. Med. Chem. Lett. 2005; 15:4854, Bioorg. Med. Chem. Lett. 2006; 16:267 and J. Med. Chem. 2006; 49:1239. Deuterated compounds of Formula (I) have not yet been described.

### BRIEF DESCRIPTION OF THE DRAWING

FIG. 1 depicts a representative result of an experiment wherein Example 9 is combined with the nucleoside analogue EIDD-1931 (CAS: 3258-02-4). The data shows a synergistic antiviral effect on SARS-CoV-2 at different doses.

### SUMMARY OF THE INVENTION

The present invention relates to compounds according to Formula (I)

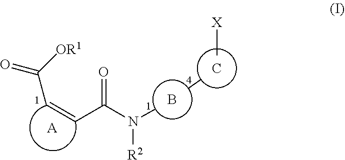

or an enantiomer, diastereomer, tautomer, prodrug, solvate, or pharmaceutically acceptable salt thereof, wherein cycle A, cycle B, cycle C, X, R¹ and R² are defined as in claim 1,
provided that at least one hydrogen in A, B, C, R² and/or X is replaced by deuterium and provided, that the level of deuterium incorporation at each substituent designated as deuterium is at least 52.5%.

The compounds of the present invention have a similar or better DHODH inhibitory activity compared to the known DHODH inhibitors. Furthermore, the compounds of the present invention exhibit an advantageous stability or pharmacokinetic profile when used as medicament due to the replacement of hydrogen to deuterium.

Thus, the present invention further relates to a pharmaceutical composition comprising a compound according to Formula (I) and at least one pharmaceutically acceptable carrier or excipient.

The present invention is further directed to compounds according to Formula (I) for use in the prophylaxis and/or treatment of diseases mediated by DHODH.

Accordingly, the present invention relates to the prophylaxis and/or treatment of the disease, disorder, therapeutic indication or medical condition which is selected from the group comprising rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation or arthropathy. More specifically, the disease, disorder or therapeutic indication is selected from the group comprising graft versus host and host versus graft reactions, rheumatoid arthritis, multiple sclerosis, amyotrophic lateral sclerosis, lupus erythematosus, inflammatory bowel disease, cancer, COVID-19, ulcerative colitis, Crohn's disease, primary sclerosing cholangitis and psoriasis.

### DETAILED DESCRIPTION OF THE INVENTION

Compound 2-((3-fluoro-3′-methoxy-[1,1′-biphenyl]-4-yl)carbamoyl)cyclopent-1-ene-1-carboxylic acid, also known as vidofludimus is an orally administered DHODH inhibitor. The calcium salt of vidofludimus is known as IMU-838. IMU-838 is currently in a Phase 2 clinical trial for the treatment of rrMS, ulcerative colitis, primary sclerosing cholangitis and COVID-19.

Compound 3-((2,3,5,6-tetrafluoro-3′-(trifluoromethoxy)-[1,1′-biphenyl]-4-yl)carbamoyl)thiophene-2-carboxylic acid, also known as PP-001 is a topically administered DHODH inhibitor. PP-001 is currently in clinical trials for the treatment of keratoconjunctivitis and non-infectious uveitis.

Vidofludimus, IMU-838 and PP-001 has generally been well-tolerated in several clinical trials. Despite the potential beneficial activities of vidofludimus, IMU-838 and PP-001, there is a continuing need for new compounds to treat the aforementioned diseases and conditions that have improved drug metabolism and pharmacokinetic (DMPK) properties. Improved DMPK properties have the potential to result in positive changes in safety profile, efficacy and tolerability of compounds.

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but include any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls.

The desired properties of a DHODH inhibitor can be yielded with compounds that follow the structural pattern represented by Formula (I):

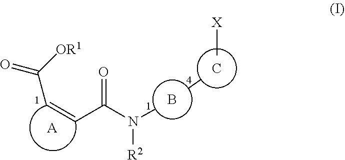

or an enantiomer, diastereomer, tautomer, prodrug, solvate, or pharmaceutically acceptable salt thereof, wherein
A is selected from a 5-membered heteroaryl, cyclopentenyl and heterocyclopentenyl, having one or more hydrogen atoms optionally replaced by deuterium,
said A is unsubstituted or substituted with 1 to 5 substituents independently selected from the group consisting of halogen, CN, NO₂, oxo, OH, C₁₋₄-alkyl, O—C₁₋₄-alkyl, fluoro-C₁₋₄-alkyl and O-fluoro-C₁₋₄-alkyl, CO₂H and SO₃H, having one or more hydrogen atoms in alkyl optionally replaced by deuterium;
B is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S,
   wherein cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR²⁷, C₀₋₆-alkylene-(3- to 6-membered cycloalkyl), C₀₋₆-alkylene-(3- to 6-membered heterocycloalkyl), C₀₋₆-alkylene-S(═O)ₙ(═NR²⁹)ₘR²⁷, C₀₋₆-alkylene-NR²⁷S(═O)ₓ(═NR²⁹)_{y}R²⁷, C₀₋₆-alkylene-S(═O)ₓ(═NR²⁹)_{y}NR²⁷R²⁸, C₀₋₆-alkylene-NR²⁷ S(═O)(═NR²⁹)_{y}NR²⁷R²⁸, C₀₋₆-alkylene-CO₂R²⁷, C₀₋₆-alkylene-O—COR²⁷, C₀₋₆-alkylene-CONR²⁷R²⁸, C₀₋₆-alkylene-NR²⁷—COR²⁷, C₀₋₆-alkylene-NR²⁷—CONR²⁷R²⁸, C₀₋₆-alkylene-O—CONR²⁷R²⁸, C₀₋₆-alkylene-NR²⁷—CO₂R²⁷, C₀₋₆-alkylene-NR²⁷R²⁸,
      wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl;
      and wherein optionally two adjacent substituents in the aryl or heteroaryl moiety form a 5- to 8-membered partially unsaturated cycle optionally containing 1 to 3 heteroatoms independently selected from O, S or N,
         wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
and wherein the residue —NR² on ring B is in a 1,4-orientation with respect to ring C, ring B or its substituents having one or more hydrogen atoms optionally replaced by deuterium;
C is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S,
   wherein cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR³³)ₘR³¹, C₀₋₆-alkylene-NR³¹S(═O)ₓ(═NR³³)_{y}R³¹, C₀₋₆-alkylene-S(═O)ₓ(═NR³³)_{y}NR³¹R³², C₀₋₆-alkylene-NR³¹ S(═O)ₓ(═NR³³)_{y}NR³¹R³², C₀₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹, C₀₋₆-alkylene-NR³¹R³²
      wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl;
   and wherein optionally two adjacent substituents in the aryl or heteroaryl moiety form a 5- to 8-membered partially unsaturated cycle optionally containing 1 to 3 heteroatoms independently selected from O, S or N,
      wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
ring C or its substituents having one or more hydrogen atoms optionally replaced by deuterium;
X is selected from H, D, halogen, —CN, —NO₂, C₁₋₆-alkyl, —O—C₁₋₆-alkyl, O-halo-C₁₋₆-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR⁴³)ₘR⁴¹, C₀₋₆-alkylene-NR⁴¹S(═O)ₓ(═NR⁴³)_{y}R⁴¹, C₀₋₆-alkylene-S(═O)ₓ(═NR⁴³)_{y}NR⁴¹R⁴², C₀₋₆-alkylene-NR⁴¹S(═O)ₓ(═NR⁴³)_{y}NR⁴¹R⁴², C₀₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹, C₀₋₆-alkylene-NR⁴¹R⁴², wherein heterocycloalkyl comprises 1, 2, 3 or 4 heteroatoms independently selected from N, O, or S,
   wherein alkyl, alkylene, cycloalkyl and heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
X or its substituents having one or more hydrogen atoms optionally replaced by deuterium;
R¹ is selected from H and D;
R² is selected from H and C₁₋₆-alkyl,
   wherein alkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R² or its substituents having one or more hydrogen atoms optionally replaced by deuterium;
R²⁷, R²⁸, R³¹, R³², R⁴¹, R⁴² are independently selected from H, C₁₋₆-alkyl, 3- to 6-membered cycloalkyl or 3- to 6-membered heterocycloalkyl,
   wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R²⁷ and/or R²⁸ and/or R³¹ and/or R³² and/or R⁴¹ and/or R⁴² or its substituents, respectively, having one or more hydrogen atoms optionally replaced by deuterium;
or R²⁷ and R²⁸, R³¹ and R³², R⁴¹ and R⁴², respectively, when taken together with the nitrogen to which they are attached complete a 3- to 6-membered cycle containing carbon atoms and optionally containing 1 or 2 heteroatoms selected from O, S or N; and
   wherein this cycle is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl,
R²⁷ and/or R^{2′} and/or R³¹ and/or R³² and/or R⁴¹ and/or R⁴² or its substituents, respectively, having one or more hydrogen atoms optionally replaced by deuterium;
R²⁹, R³³, R⁴³ are independently selected from H, —CN, —NO₂, C₁₋₆-alkyl, —CO—O—C₁₋₆-alkyl, 3- to 6-membered cycloalkyl or 3- to 6-membered heterocycloalkyl,
   wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R²⁹ and/or R³³ and/or R⁴³ or its substituents, respectively, having one or more hydrogen atoms optionally replaced by deuterium;
n, m, x, y are independently selected from 0 to 2;
   with the proviso that the sum of integer m and n for the residue linked to the same sulfur atom is independently selected from 0 to 2;
   with the proviso that the sum of integer x and y for the residue linked to the same sulfur atom is independently selected from 1 or 2;
provided that at least one hydrogen in A, B, C, R², R²⁷, R²⁸, R²⁹, R³¹, R³², R³³, R⁴¹, R⁴², R⁴³ and/or X is replaced by deuterium;
provided, that the level of deuterium incorporation at each substituent designated as deuterium is at least 52.5%.

In a more particular embodiment the compound is represented by Formula (I), a solvate or pharmaceutically acceptable salt thereof, wherein
R¹ is H and R² is H.

In a more particular embodiment in combination with any of the above or below embodiments the compound is represented by Formula (I), wherein

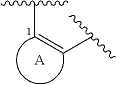

is selected from

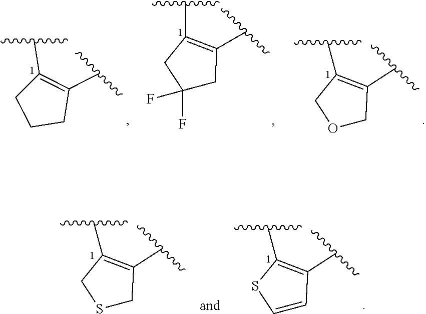 [SMILES: *C1=C(*)CCC1 ; *C1=C(*)CC(F)(F)C1 ; *C1=C(*)COC1 ; *C1=C(*)CSC1 ; *c1ccsc1*]

In a similar particular embodiment in combination with any of the above or below embodiments the compound is represented by Formula (I), wherein

—NR²B is selected from

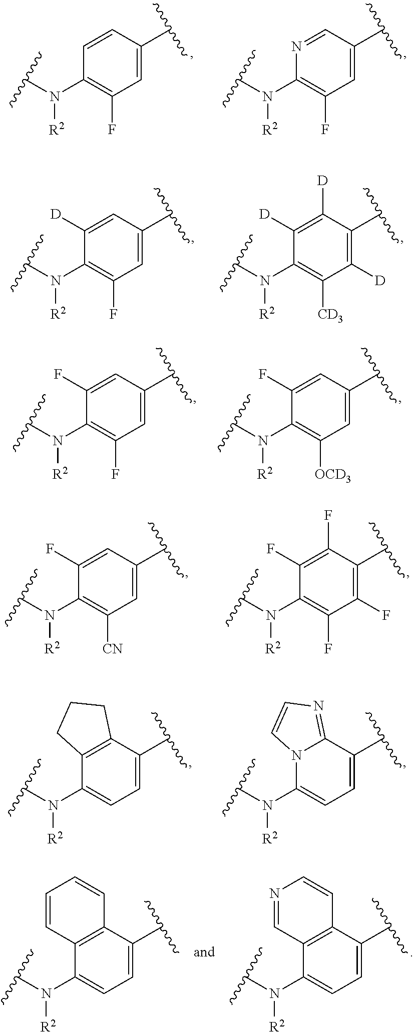

In a similar particular embodiment in combination with any of the above or below embodiments the compound is represented by Formula (I), wherein
C is phenyl, pyridyl or thiazolyl,
   wherein phenyl, pyridyl or thiazolyl is unsubstituted or substituted with 1 to 4 substituents independently selected from the group consisting of D and F;
X is selected from D, F, Cl, —CN, OH, C₁₋₄-alkyl, O—C₁₋₄-alkyl, fluoro-C₁₋₄-alkyl, O-fluoro-C₁₋₄-alkyl, having one or more hydrogen atoms optionally replaced by deuterium.

In a more particular embodiment in combination with any of the above or below embodiments the compound is represented by Formula (I), wherein

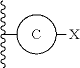

is selected from

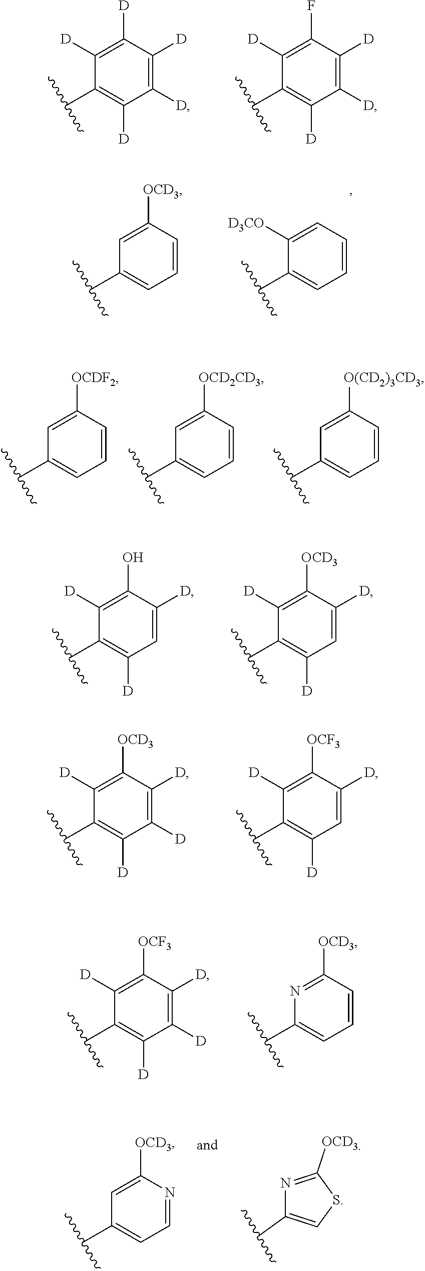

In a more particular embodiment in combination with any of the above or below embodiments the compound is represented by Formula (I), wherein

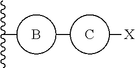

is selected from

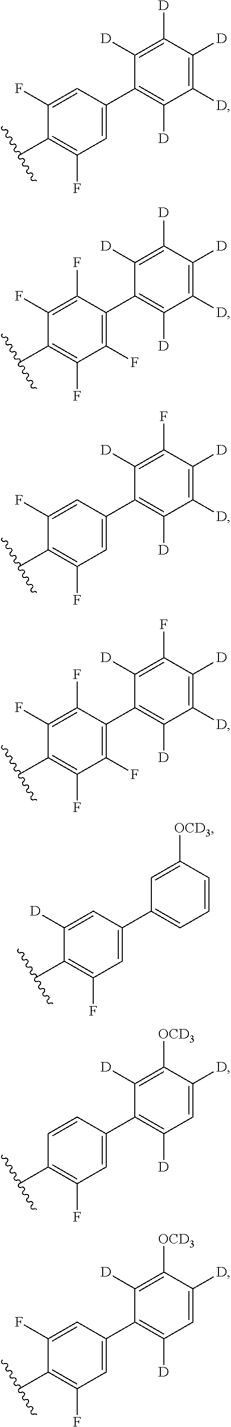

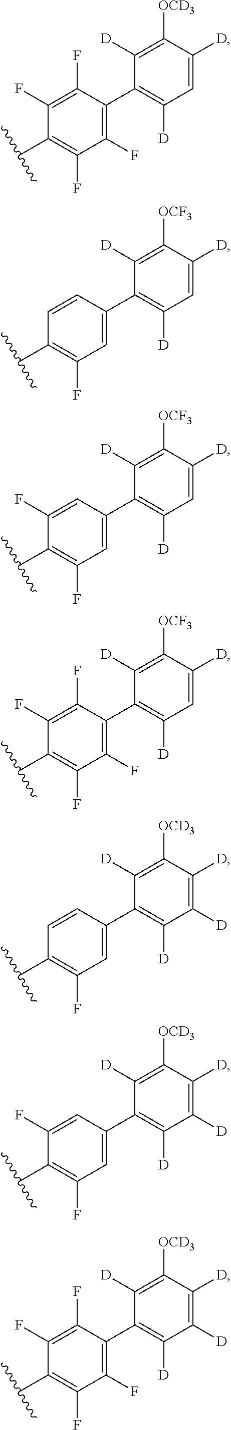

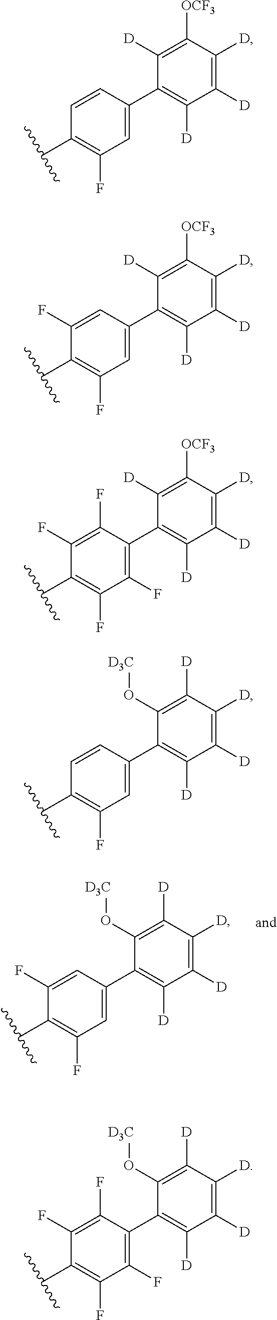

In a more particular embodiment in combination with any of the above or below embodiments the compound is represented by Formula (I), wherein
R¹ is H and R² is H;

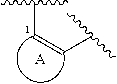

is selected from

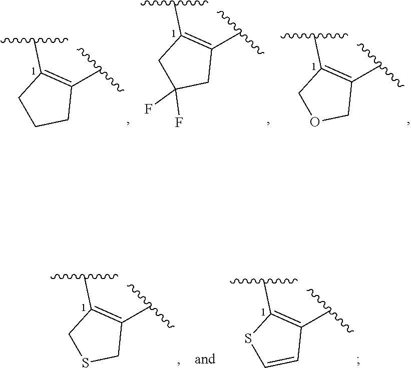 [SMILES: *C1=C(*)CCC1 ; *C1=C(*)CC(F)(F)C1 ; *C1=C(*)COC1 ; *C1=C(*)CSC1 ; *c1ccsc1*]

—NR²B is selected from

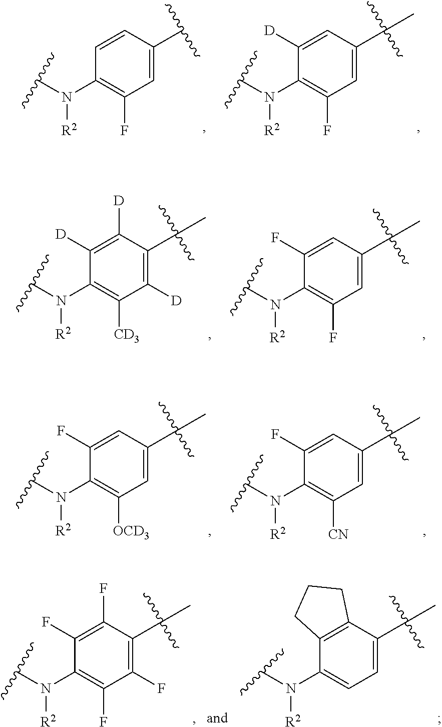

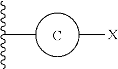

is selected from

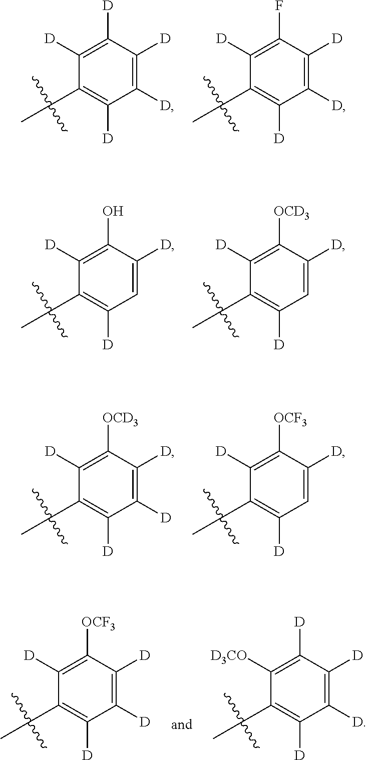

In a most particular embodiment the compound is selected from

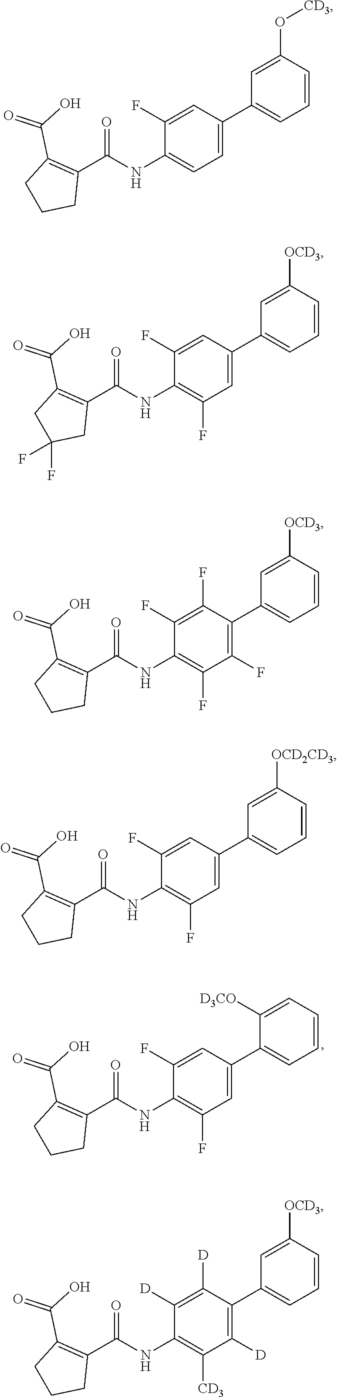

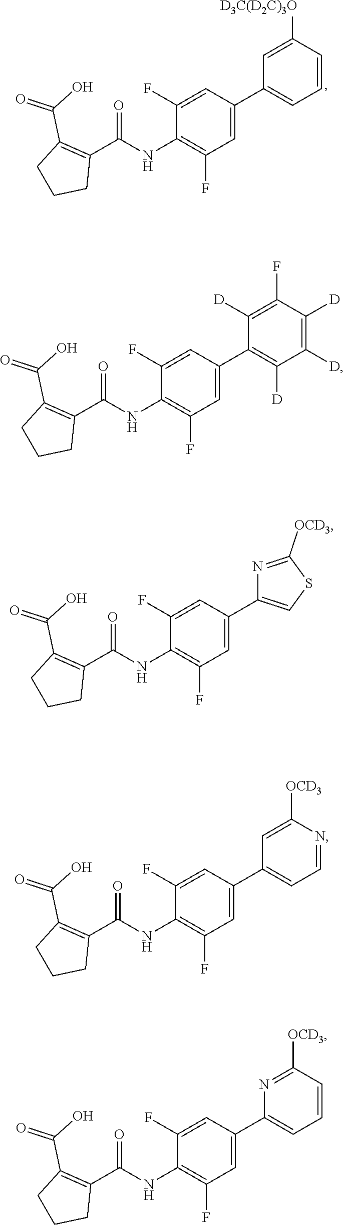

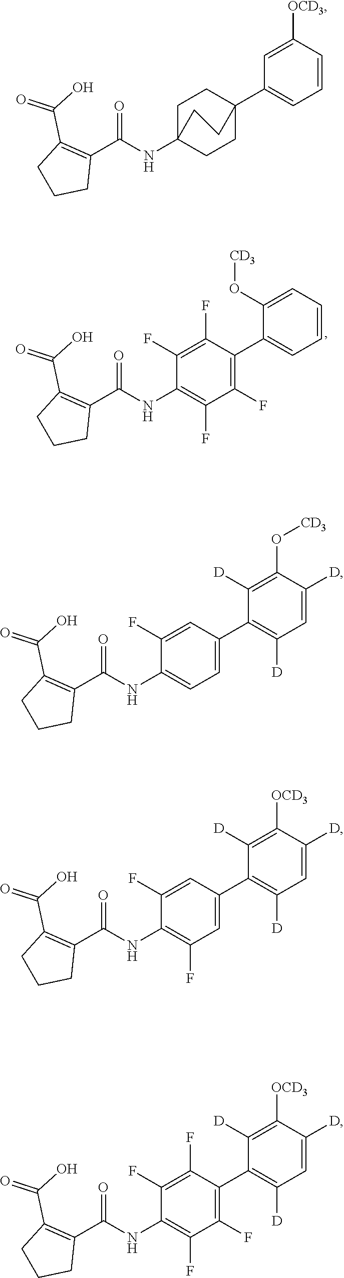

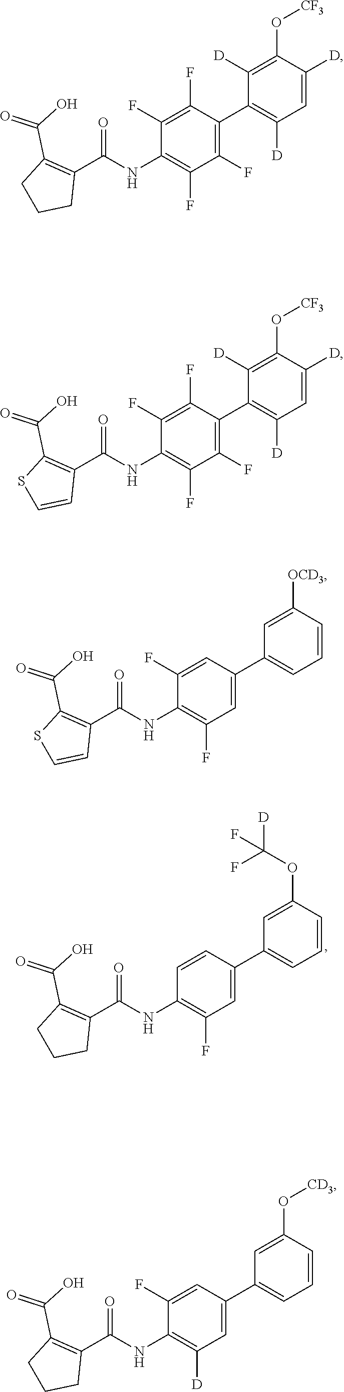

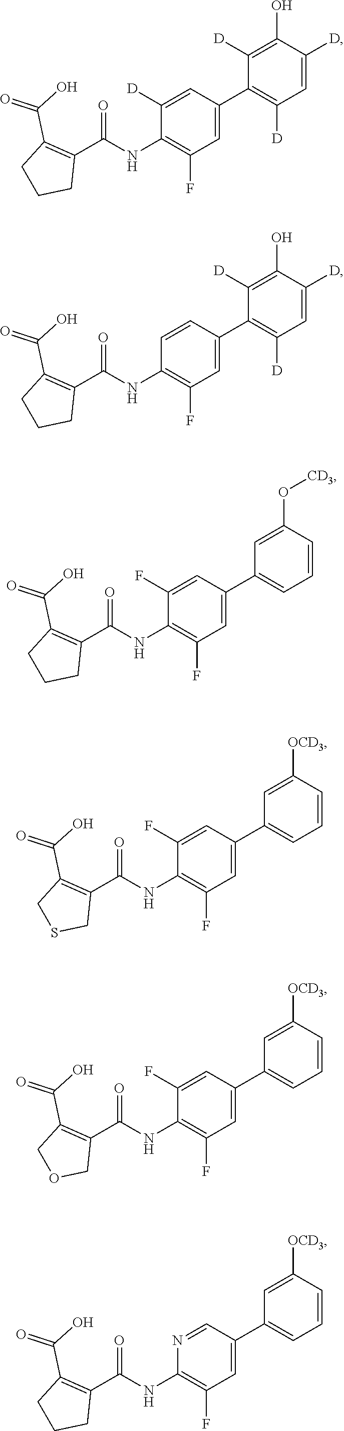

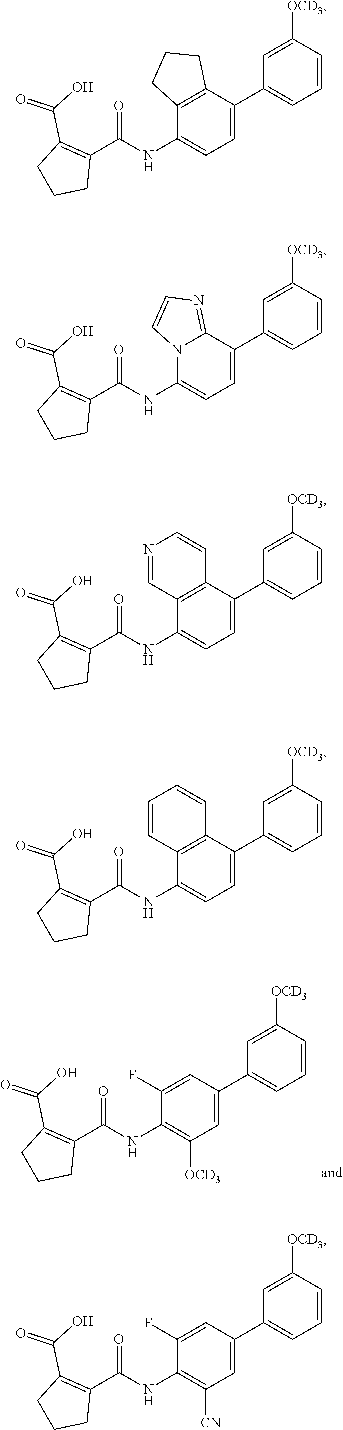

or a solvate or pharmaceutically acceptable salt thereof.

The invention also provides the compound of the present invention for the use as a medicament. Also provides is the compound of the present invention for use in the prophylaxis and/or treatment of diseases, disorders, therapeutic indications or medical conditions amenable for treatment with DHODH inhibitors.

Also provided is the compound of the present invention for use in the prophylaxis and/or treatment of a DHODH mediated disease selected from rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation or arthropathy.

More specifically, the invention relates to a compound of the present invention for use wherein the disease, disorder or therapeutic indication is selected from the group comprising graft versus host and host versus graft reactions, rheumatoid arthritis, multiple sclerosis, amyotrophic lateral sclerosis, lupus erythematosus, inflammatory bowel disease, cancer, COVID-19, influenza, ulcerative colitis, Crohn's disease, primary sclerosing cholangitis and psoriasis.

Also provided is a pharmaceutical composition comprising a compound of the present invention and a pharmaceutically acceptable carrier or excipient.

Also provided is a pharmaceutical composition comprising a compound of the present invention and a pharmaceutically acceptable carrier or excipient and further comprising one or more additional therapeutic agents selected from antiviral agents, anti-inflammatory agents, immunosuppressive and/or immunomodulatory agents, steroids, non-steroidal anti-inflammatory agents, antihistamines, analgesics and suitable mixtures thereof.

In addition, the desired properties of a DHODH inhibitor can be yielded with compounds that follow the structural pattern represented by Formula (I):

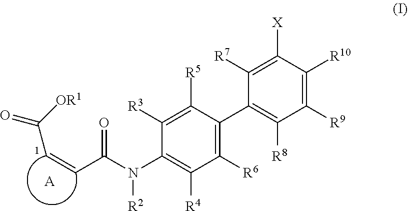

or an enantiomer, diastereomer, tautomer, prodrug, solvate, or pharmaceutically acceptable salt thereof, wherein
R¹ and R² are independently selected from H and D;
R³, R⁴, R⁵ and R⁶ are independently selected from H, D, halogen, CN, C₁₋₄-alkyl, O—C₁₋₄-alkyl, fluoro-C₁₋₄-alkyl and O-fluoro-C₁₋₄-alkyl, having one or more hydrogen atoms in alkyl optionally replaced by deuterium;
R⁷, R¹, R⁹ and R¹⁰ are independently selected from H, D, halogen, CN, C₁₋₄-alkyl, O—C₁₋₄-alkyl, fluoro-C₁₋₄-alkyl and O-fluoro-C₁₋₄-alkyl, having one or more hydrogen atoms in alkyl optionally replaced by deuterium;
X is selected from H, D, OH, OD, S(═O)_{y}R¹¹ and OR¹¹;
R¹¹ is selected from C₁₋₄-alkyl, C₃₋₄-cycloalkyl and fluoro-C₁₋₄-alkyl, having one or more hydrogen atoms in alkyl optionally replaced by deuterium;
y is 0 to 2;

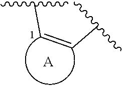

is selected from a 5-membered heteroaryl, cyclopentenyl and heterocyclopentenyl, having one or more hydrogen atoms optionally replaced by deuterium,
said A is unsubstituted or substituted with 1 to 5 substituents independently selected from the group consisting of halogen, CN, NO₂, oxo, OH, C₁₋₄-alkyl, O—C₁₋₄-alkyl, fluoro-C₁₋₄-alkyl and O-fluoro-C₁₋₄-alkyl, CO₂H and SO₃H, having one or more hydrogen atoms in alkyl optionally replaced by deuterium; provided that at least one hydrogen in R³, R⁴, R⁵, R⁶, R⁷, R⁸, R⁹, R¹⁰, R¹¹, X and/or A is replaced by deuterium.

In a special embodiment the compound is represented by Formula (I), or a prodrug, pharmaceutically acceptable salt or solvate thereof, wherein
R¹ and R² are independently selected from H and D;
R³, R⁴, R⁵ and R⁶ are independently selected from H, D, F and Cl;
R⁷, R⁸, R⁹ and R¹⁰ are independently selected from H, D and F;
X is selected from H, D, OH, OD and OR¹¹;
R¹¹ is selected from C₁₋₄-alkyl and fluoro-C₁₋₄-alkyl, having one or more hydrogen atoms in alkyl optionally replaced by deuterium;

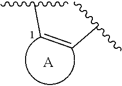

is selected from

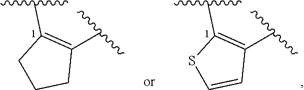 [SMILES: *C1=C(*)CCC1 ; *c1ccsc1*]

having one or more hydrogen atoms optionally replaced by deuterium;
provided that at least one hydrogen in R³, R⁴, R⁵, R⁶, R⁷, R⁸, R⁹, R¹⁰, R¹¹ and/or A is replaced by deuterium.

In a particular embodiment the compound is represented by Formula (I), or a prodrug, pharmaceutically acceptable salt or solvate thereof, wherein
R³ is F;
R⁴, R⁵ and R⁶ are independently selected from H, D and F;
R⁷, R⁸, R⁹ and R¹⁰ are independently selected from H and D;
X is selected from OH, OD and OR¹¹;
R¹¹ is selected from CH₃, CD₃, CHF₂, CDF₂ and CF₃;

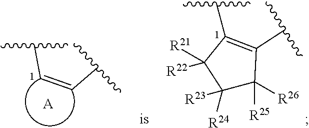

R²¹, R²², R²³, R²⁴, R²⁵ and R²⁶ are independently selected from H and D;
provided that at least one hydrogen in R⁴, R⁵, R⁶, R⁷, R⁸, R⁹, R¹⁰, R¹¹, R²¹, R²², R²³, R²⁴, R²⁵ and/or R²⁶ is replaced by deuterium.

In a more particular embodiment the compound is represented by Formula (I), or a prodrug, pharmaceutically acceptable salt or solvate thereof, wherein
R⁴, R⁵ and R⁶ are independently selected from H and D;
X is OR¹¹;
R¹¹ is selected from CH₃ and CD₃;
provided that at least one hydrogen in R⁴, R⁵, R⁶, R⁷, R⁸, R⁹, R¹⁰, R¹¹, R²¹, R²², R²³, R²⁴, R²⁵ and/or R²⁶ is replaced by deuterium.

In a more particular embodiment in combination with any of the above or below embodiments, R¹¹ is CD₃.

In a more particular embodiment, the compound is selected from

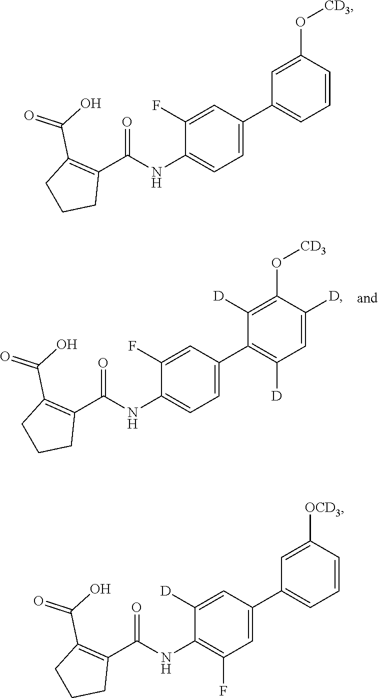

or a solvate or pharmaceutically acceptable salt thereof.

In a most particular embodiment, the compound is selected from

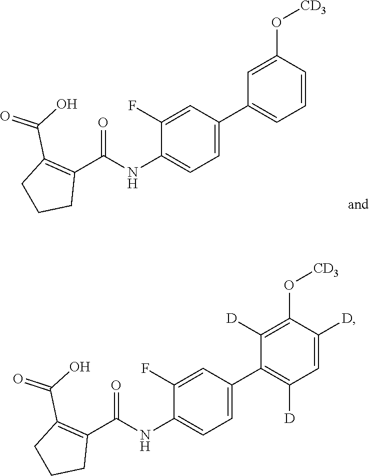

or a solvate or pharmaceutically acceptable salt thereof.

In a similar particular embodiment the compound is represented by Formula (I), or a prodrug, pharmaceutically acceptable salt or solvate thereof, wherein
R¹ and R² are independently selected from H and D;
R³, R⁴, R⁵ and R⁶ are independently selected from H, D, F and Cl;
R⁷, R⁸, R⁹ and R¹⁰ are independently selected from H, D and F;
X is OR¹¹;
R¹¹ is selected from C₁₋₄-alkyl and fluoro-C₁₋₄-alkyl, having one or more hydrogen atoms in alkyl optionally replaced by deuterium;

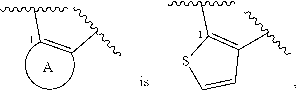

having one or more hydrogen atoms optionally replaced by deuterium; provided that at least one hydrogen in R³, R⁴, R⁵, R⁶, R⁷, R⁸, R⁹, R¹⁰, R¹¹ and/or A is replaced by deuterium.

In a similar most particular embodiment, the compound is

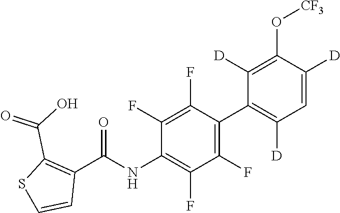 [SMILES: [2H]c1cc([2H])c(-c2c(F)c(F)c(NC(=O)c3ccsc3C(=O)O)c(F)c2F)c([2H])c1OC(F)(F)F]

or a solvate or pharmaceutically acceptable salt thereof.

The invention also provides the compound of the present invention for the use as a medicament.

Also provides is the compound of the present invention for use in the prophylaxis and/or treatment of diseases, disorders, therapeutic indications or medical conditions amenable for treatment with DHODH inhibitors.

Also provided is the compound of the present invention for use in the prophylaxis and/or treatment of a DHODH mediated disease selected from rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation or arthropathy.

More specifically, the invention relates to a compound of the present invention for use wherein the disease, disorder or therapeutic indication is selected from the group comprising graft versus host and host versus graft reactions, rheumatoid arthritis, multiple sclerosis, amyotrophic lateral sclerosis, lupus erythematosus, inflammatory bowel disease, cancer, COVID-19, ulcerative colitis, Crohn's disease, primary sclerosing cholangitis and psoriasis.

Also provided is a pharmaceutical composition comprising a compound of the present invention and a pharmaceutically acceptable carrier or excipient.

Also provided is a pharmaceutical composition comprising a compound of the present invention and a pharmaceutically acceptable carrier or excipient and further comprising one or more additional therapeutic agents selected from anti-inflammatory agents, immunosuppressive and/or immunomodulatory agents, steroids, non-steroidal anti-inflammatory agents, antihistamines, analgesics and suitable mixtures thereof.

In certain embodiments the present invention relates to a compound of Formula (I) as described in the following items:
1. A compound according to Formula (I):

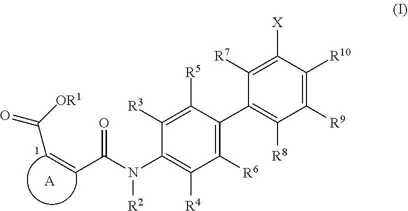

or an enantiomer, diastereomer, tautomer, prodrug, solvate, or pharmaceutically acceptable salt thereof, wherein
R¹ and R² are independently selected from H and D;
R³, R⁴, R⁵ and R⁶ are independently selected from H, D, halogen, CN, C₁₋₄-alkyl, O—C₁₋₄-alkyl, fluoro-C₁₋₄-alkyl and O-fluoro-C₁₋₄-alkyl, having one or more hydrogen atoms in alkyl optionally replaced by deuterium;
R⁷, R⁸, R⁹ and R¹⁰ are independently selected from H, D, halogen, CN, C₁₋₄-alkyl, O—C₁₋₄-alkyl, fluoro-C₁₋₄-alkyl and O-fluoro-C₁₋₄-alkyl, having one or more hydrogen atoms in alkyl optionally replaced by deuterium;
X is selected from H, D, OH, OD, S(═O)_{y}R¹¹ and OR¹¹;
R¹¹ is selected from C₁₋₄-alkyl, C₃₋₄-cycloalkyl and fluoro-C₁₋₄-alkyl, having one or more hydrogen atoms in alkyl optionally replaced by deuterium;
y is 0 to 2;

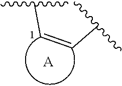

is selected from a 5-membered heteroaryl, cyclopentenyl and heterocyclopentenyl, having one or more hydrogen atoms optionally replaced by deuterium,
said A is unsubstituted or substituted with 1 to 5 substituents independently selected from the group consisting of halogen, CN, NO₂, oxo, OH, C₁₋₄-alkyl, O—C₁₋₄-alkyl, fluoro-C₁₋₄-alkyl and O-fluoro-C₁₋₄-alkyl, CO₂H and SO₃H, having one or more hydrogen atoms in alkyl optionally replaced by deuterium; provided that at least one hydrogen in R³, R⁴, R⁵, R⁶, R⁷, R⁸, R⁹, R¹⁰, R¹¹, X and/or A is replaced by deuterium.
2. A compound according to item 1 with Formula (I), or a prodrug, pharmaceutically acceptable salt or solvate thereof, wherein
R¹ and R² are independently selected from H and D;
R³, R⁴, R⁵ and R⁶ are independently selected from H, D, F and Cl;
R⁷, R¹, R⁹ and R¹⁰ are independently selected from H, D and F;
X is selected from H, D, OH, OD and OR¹¹;
R¹¹ is selected from C₁₋₄-alkyl and fluoro-C₁₋₄-alkyl, having one or more hydrogen atoms in alkyl optionally replaced by deuterium;

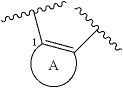

is selected from

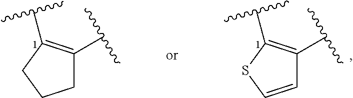 [SMILES: *C1=C(*)CCC1 ; *c1ccsc1*]

having one or more hydrogen atoms optionally replaced by deuterium;
provided that at least one hydrogen in R³, R⁴, R⁵, R⁶, R⁷, R⁸, R⁹, R¹⁰, R¹¹ and/or A is replaced by deuterium.
3. A compound according to item 2 with Formula (I), or a prodrug, pharmaceutically acceptable salt or solvate thereof, wherein
R³ is F;
R⁴, R⁵ and R⁶ are independently selected from H, D and F;
R⁷, R⁸, R⁹ and R¹⁰ are independently selected from H and D;
X is selected from OH, OD and OR¹¹;
R¹¹ is selected from CH₃, CD₃, CHF₂, CDF₂ and CF₃;

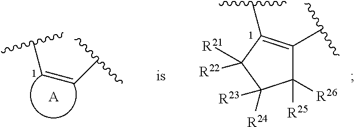

R²¹, R²², R²³, R²⁴, R²⁵ and R²⁶ are independently selected from H and D;
provided that at least one hydrogen in R⁴, R⁵, R⁶, R⁷, R⁸, R⁹, R¹⁰, R¹¹, R²¹, R²², R²³, R²⁴, R²⁵ and/or R²⁶ is replaced by deuterium.
4. A compound according to item 3 with Formula (I), or a prodrug, pharmaceutically acceptable salt or solvate thereof, wherein
R⁴, R⁵ and R⁶ are independently selected from H and D;
X is OR¹¹;
R¹¹ is selected from CH₃ and CD₃;
provided that at least one hydrogen in R⁴, R⁵, R⁶, R⁷, R⁸, R⁹, R¹⁰, R¹¹, R²¹, R²², R²³, R²⁴, R²⁵ and/or R²⁶ is replaced by deuterium.
5. A compound according to item 1 to 4, wherein R¹¹ is CD₃.
6. A compound according to any one of the preceding items, which is selected from

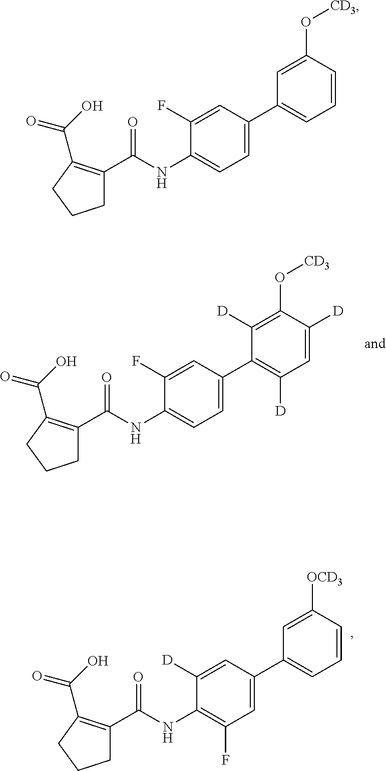

or a solvate or pharmaceutically acceptable salt thereof.
7. A compound according to item 1 or 2 with the Formula (I) or a prodrug, pharmaceutically acceptable salt or solvate thereof, wherein
R¹ and R² are independently selected from H and D;
R³, R⁴, R⁵ and R⁶ are independently selected from H, D, F and Cl;
R⁷, R¹, R⁹ and R¹⁰ are independently selected from H, D and F;
X is OR¹¹;
R¹¹ is selected from C₁₋₄-alkyl and fluoro-C₁₋₄-alkyl, having one or more hydrogen atoms in alkyl optionally replaced by deuterium;

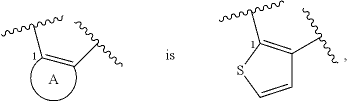

having one or more hydrogen atoms optionally replaced by deuterium;
provided that at least one hydrogen in R³, R⁴, R⁵, R⁶, R⁷, R⁸, R⁹, R¹⁰, R¹¹ and/or A is replaced by deuterium.
8. A compound according to any one of the preceding items, which is

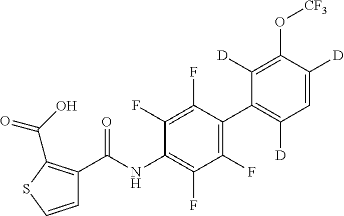 [SMILES: [2H]c1cc([2H])c(-c2c(F)c(F)c(NC(=O)c3ccsc3C(=O)O)c(F)c2F)c([2H])c1OC(F)(F)F]

or a solvate or pharmaceutically acceptable salt thereof.
9. A compound according to any one of the preceding items for the use as a medicament.
10. A compound according to any one of item 1 to 9 for use in the prophylaxis and/or treatment of diseases, disorders, therapeutic indications or medical conditions amenable for treatment with DHODH inhibitors.
11. A compound for use according to item 10 wherein the disease, disorder, therapeutic indication or medical condition is selected from the group comprising rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation or arthropathy.
12. A compound for use according to item 11 wherein the disease, disorder or therapeutic indication is selected from the group comprising graft versus host and host versus graft reactions, rheumatoid arthritis, multiple sclerosis, amyotrophic lateral sclerosis, lupus erythematosus, inflammatory bowel disease, cancer, COVID-19, ulcerative colitis, Crohn's disease, primary sclerosing cholangitis and psoriasis.
13. A pharmaceutical composition comprising a compound according to any one of item 1 to 8 and a pharmaceutically acceptable carrier or excipient.
14. A pharmaceutical composition of item 13, further comprising one or more additional therapeutic agents selected from anti-inflammatory agents, immunosuppressive and/or immunomodulatory agents, steroids, non-steroidal anti-inflammatory agents, antihistamines, analgesics and suitable mixtures thereof.
15. A compound according to Formula (I):

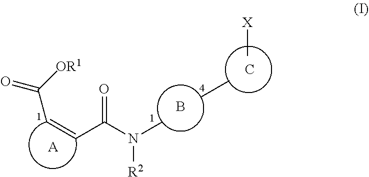

or an enantiomer, diastereomer, tautomer, prodrug, solvate, or pharmaceutically acceptable salt thereof, wherein
A is selected from a 5-membered heteroaryl, cyclopentenyl and heterocyclopentenyl, having one or more hydrogen atoms optionally replaced by deuterium,
said A is unsubstituted or substituted with 1 to 5 substituents independently selected from the group consisting of halogen, CN, NO₂, oxo, OH, C₁₋₄-alkyl, O—C₁₋₄-alkyl, fluoro-C₁₋₄-alkyl and O-fluoro-C₁₋₄-alkyl, CO₂H and SO₃H, having one or more hydrogen atoms in alkyl optionally replaced by deuterium;
B is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S,
   wherein cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR²⁷, C₀₋₆-alkylene-(3- to 6-membered cycloalkyl), C₀₋₆-alkylene-(3- to 6-membered heterocycloalkyl), C₀₋₆-alkylene-S(═O)ₙ(═NR²⁹)ₘR²⁷, C₀₋₆-alkylene-NR²¹S(═O)ₓ(═NR²⁹)_{y}R²¹, C₀₋₆-alkylene-S(═O)ₓ(═NR²⁹)_{y}NR²⁷R²⁸, C₀₋₆-alkylene-NR²⁷ S(═O)ₓ(═NR²⁹)_{y}NR²⁷R²⁸, C₀₋₆-alkylene-CO₂R²⁷, C₀₋₆-alkylene-O—COR²⁷, C₀₋₆-alkylene-CONR²⁷R²⁸, C₀₋₆-alkylene-NR²⁷—COR²⁷, C₀₋₆-alkylene-NR²⁷—CONR²⁷R²⁸, C₀₋₆-alkylene-O—CONR²⁷R²⁸, C₀₋₆-alkylene-NR²⁷—CO₂R²⁷, C₀₋₆-alkylene-NR²⁷R²⁸,
      wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl;
   and wherein optionally two adjacent substituents in the aryl or heteroaryl moiety form a 5- to 8-membered partially unsaturated cycle optionally containing 1 to 3 heteroatoms independently selected from O, S or N,
      wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
and wherein the residue —NR² on ring B is in a 1,4-orientation with respect to ring C,
ring B or its substituents having one or more hydrogen atoms optionally replaced by deuterium;
C is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S,
   wherein cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR³³)ₘR³¹, C₀₋₆-alkylene-NR³¹S(═O)ₓ(═NR³³)_{y}R³¹, C₀₋₆-alkylene-S(═O)ₓ(═NR³³)_{y}NR³¹R³², C₀₋₆-alkylene-NR³¹ S(═O)ₓ(═NR³³)_{y}NR³¹R³², C₀₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹, C₀₋₆-alkylene-NR³¹R³²
      wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl;
   and wherein optionally two adjacent substituents in the aryl or heteroaryl moiety form a 5- to 8-membered partially unsaturated cycle optionally containing 1 to 3 heteroatoms independently selected from O, S or N,
      wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
ring C or its substituents having one or more hydrogen atoms optionally replaced by deuterium;
X is selected from H, D, halogen, —CN, —NO₂, C₁₋₆-alkyl, —O—C₁₋₆-alkyl, O-halo-C₁₋₆-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR⁴³)ₘR⁴¹, C₀₋₆-alkylene-NR⁴¹S(═O)ₓ(═NR⁴³)_{y}R⁴¹, C₀₋₆-alkylene-S(═O)ₓ(═NR⁴³)_{y}NR⁴¹R⁴², C₀₋₆-alkylene-NR⁴¹S(═O)ₓ(═NR⁴³)_{y}NR⁴¹R⁴², C₀₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹, C₀₋₆-alkylene-NR⁴¹R⁴², wherein heterocycloalkyl comprises 1, 2, 3 or 4 heteroatoms independently selected from N, O, or S,
   wherein alkyl, alkylene, cycloalkyl and heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
X or its substituents having one or more hydrogen atoms optionally replaced by deuterium;
R¹ is selected from H and D;
R² is selected from H and C₁₋₆-alkyl,
   wherein alkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R² or its substituents having one or more hydrogen atoms optionally replaced by deuterium;
R²⁷, R²⁸, R³¹, R³², R⁴¹, R⁴² are independently selected from H, C₁₋₆-alkyl, 3- to 6-membered cycloalkyl or 3- to 6-membered heterocycloalkyl,
   wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R²⁷ and/or R²⁸ and/or R³¹ and/or R³² and/or R⁴¹ and/or R⁴² or its substituents, respectively, having one or more hydrogen atoms optionally replaced by deuterium;
or R²⁷ and R²⁸, R³¹ and R³², R⁴¹ and R⁴², respectively, when taken together with the nitrogen to which they are attached complete a 3- to 6-membered cycle containing carbon atoms and optionally containing 1 or 2 heteroatoms selected from O, S or N; and
   wherein this cycle is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl,
R²⁷ and/or R²⁸ and/or R³¹ and/or R³² and/or R⁴¹ and/or R⁴² or its substituents, respectively, having one or more hydrogen atoms optionally replaced by deuterium;
R²⁹, R³³, R⁴³ are independently selected from H, —CN, —NO₂, C₁₋₆-alkyl, —CO—O—C₁₋₆-alkyl, 3- to 6-membered cycloalkyl or 3- to 6-membered heterocycloalkyl,
   wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl,
   wherein heterocycloalkyl comprises 1, 2, 3 or 4 heteroatoms independently selected from N, O, or S,
R²⁹ and/or R³³ and/or R⁴³ or its substituents, respectively, having one or more hydrogen atoms optionally replaced by deuterium;
n, m, x, y are independently selected from 0 to 2;
   with the proviso that the sum of integer m and n for the residue linked to the same sulfur atom is independently selected from 0 to 2;
   with the proviso that the sum of integer x and y for the residue linked to the same sulfur atom is independently selected from 1 or 2;
provided that at least one hydrogen in A, B, C, R², R²⁷, R²⁸, R²⁹, R³¹, R³², R³³, R⁴¹, R⁴², R⁴³ and/or X is replaced by deuterium;
provided, that the level of deuterium incorporation at each substituent designated as deuterium is at least 52.5%.
16. A compound of Formula (I) according to item 15, or a solvate or pharmaceutically acceptable salt thereof, wherein
R¹ is H and R² is H.
17. A compound of Formula (I) according to item 15 or 16, wherein

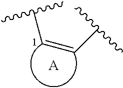

is selected from

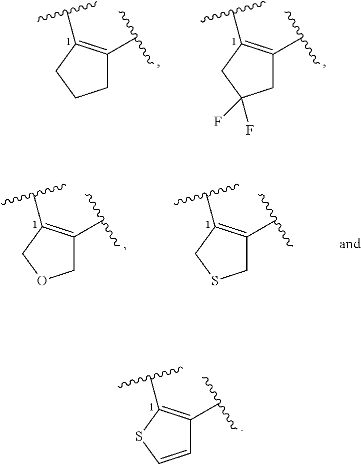 [SMILES: *C1=C(*)CCC1 ; *C1=C(*)CC(F)(F)C1 ; *C1=C(*)COC1 ; *C1=C(*)CSC1 ; *c1ccsc1*]

18. A compound of Formula (I) according to any of item 15 to 17, wherein
—NR²B is selected from

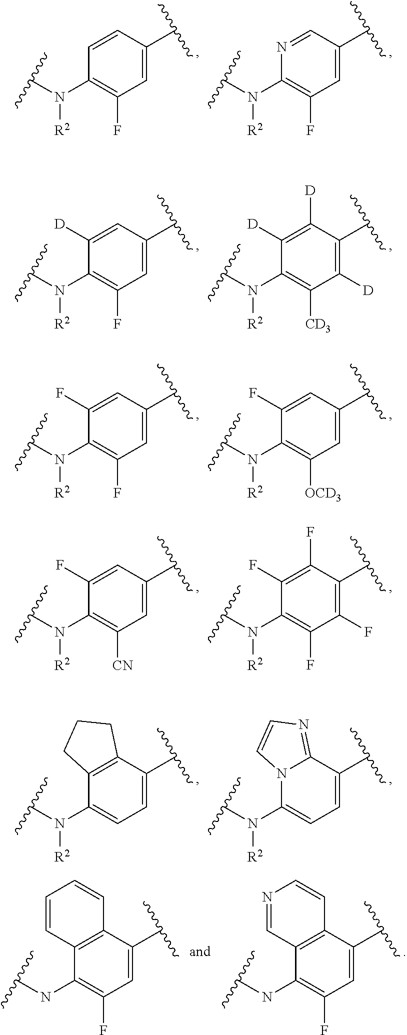

19. A compound of Formula (I) according to any of item 15 to 18, wherein C is phenyl, pyridyl or thiazolyl,
   wherein phenyl, pyridyl or thiazolyl is unsubstituted or substituted with 1 to 4 substituents independently selected from the group consisting of D and F;
X is selected from D, F, Cl, —CN, OH, C₁₋₄-alkyl, O—C₁₋₄-alkyl, fluoro-C₁₋₄-alkyl, O-fluoro-C₁₋₄-alkyl, having one or more hydrogen atoms optionally replaced by deuterium.
20. A compound of Formula (I) according to any of item 15 to 19, wherein

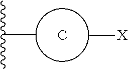

is selected from

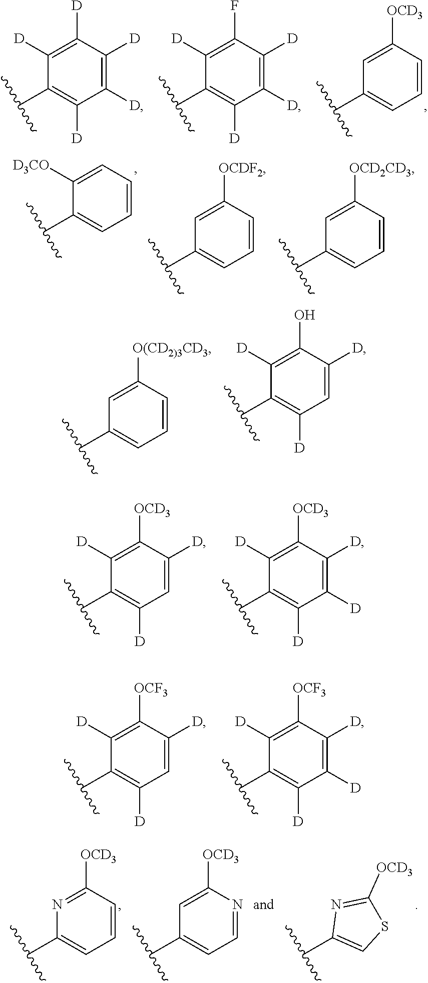

21. A compound of Formula (I) according to any of item 15 to 20, wherein

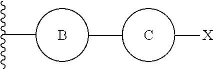

is selected from

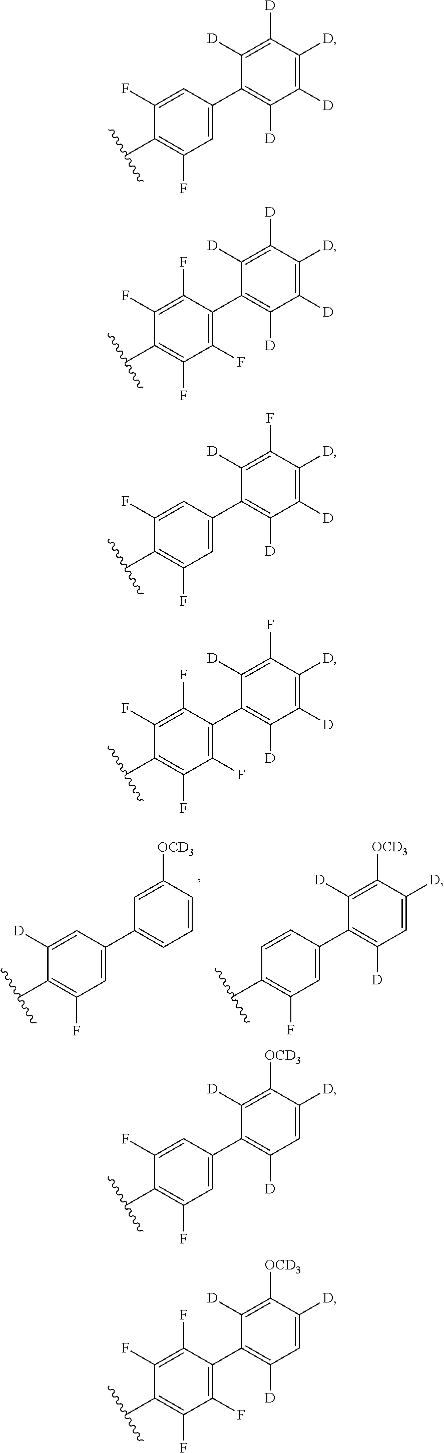

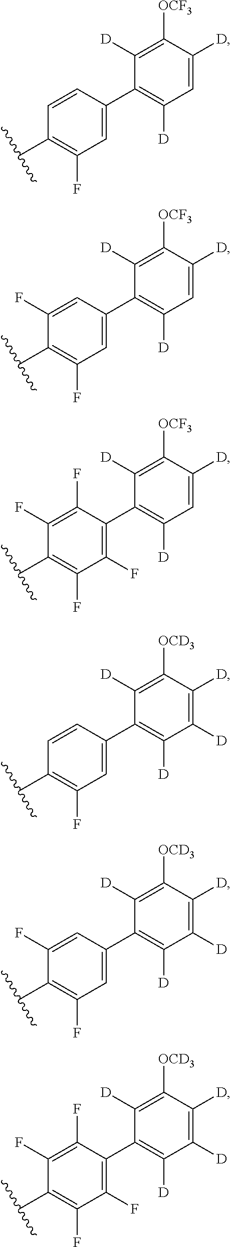

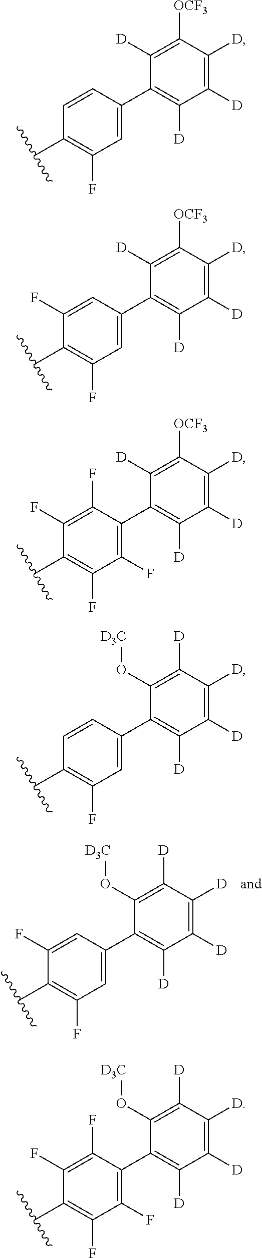

22. A compound of Formula (I) according to any of item 15 to 21, wherein
R¹ is H and R² is H;

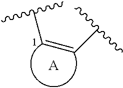

is selected from

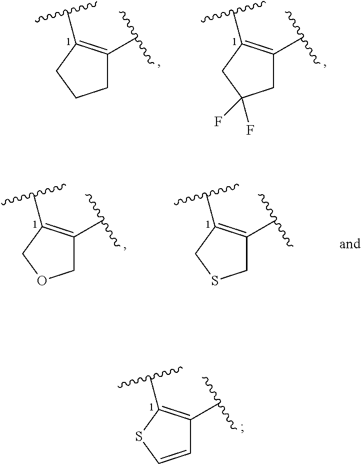 [SMILES: *C1=C(*)CCC1 ; *C1=C(*)CC(F)(F)C1 ; *C1=C(*)COC1 ; *C1=C(*)CSC1 ; *c1ccsc1*]

—NR²B is selected from

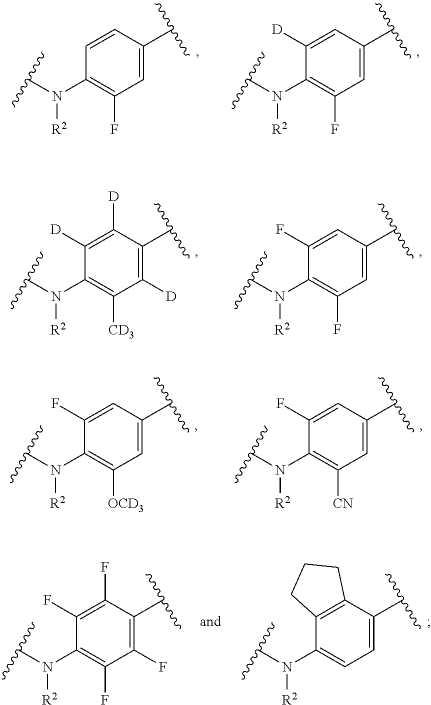

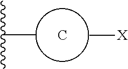

is selected from

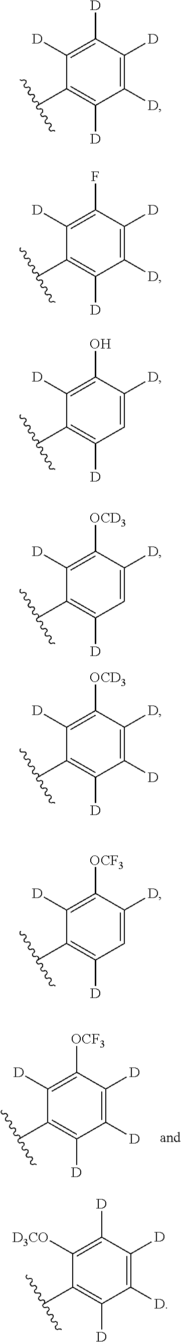

23. A compound of Formula (I) according to any of item 15 to 22, which is selected from

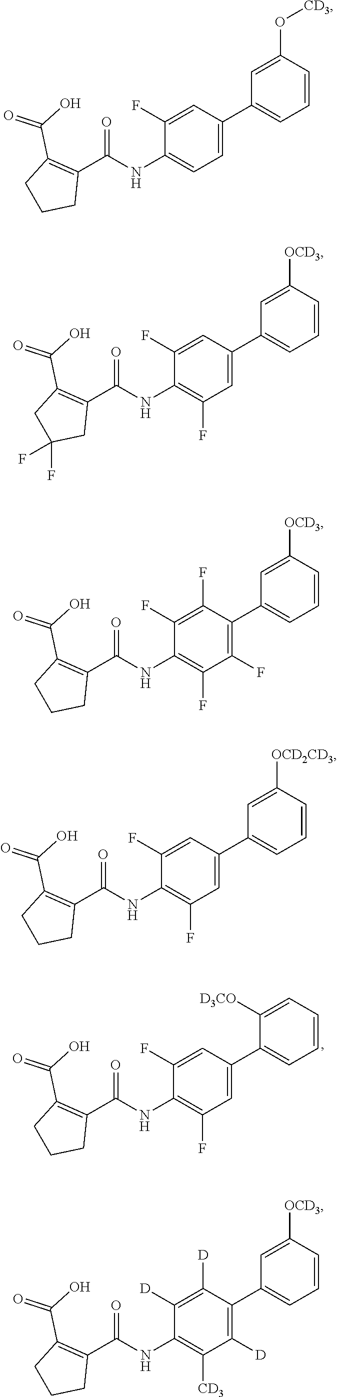

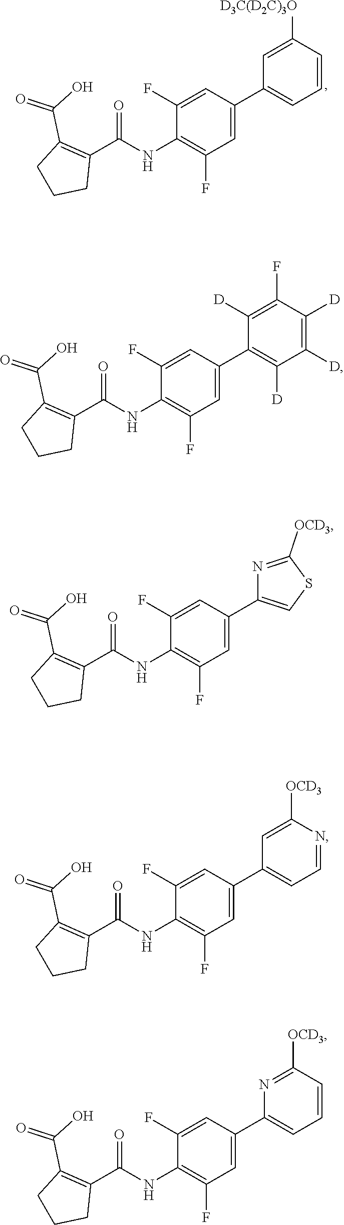

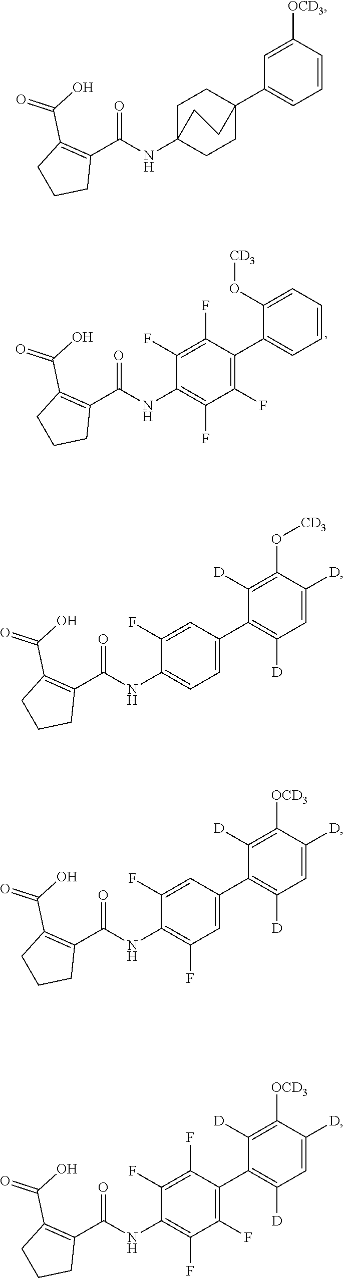

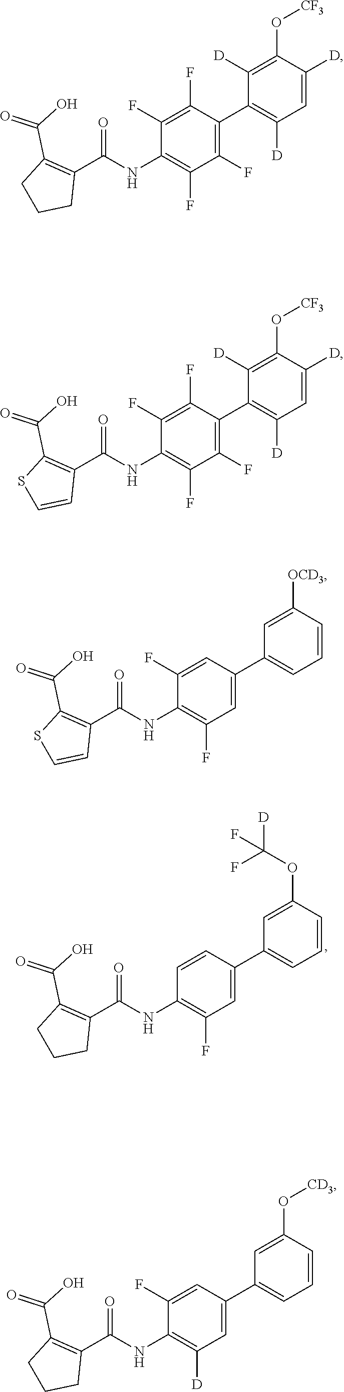

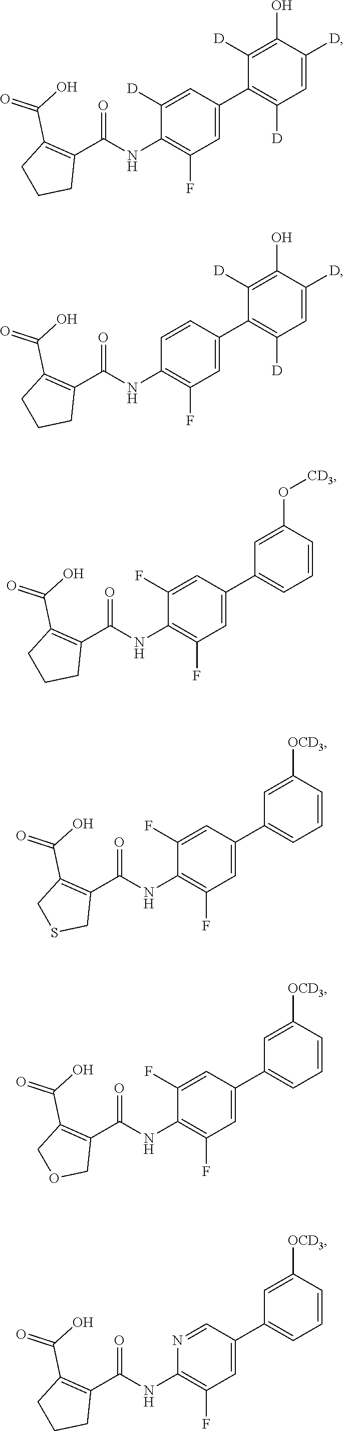

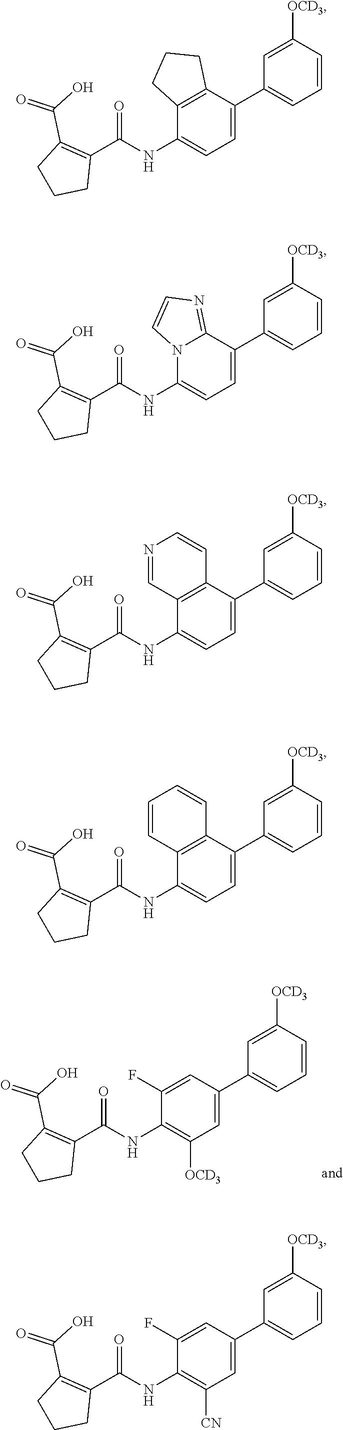

or a solvate or pharmaceutically acceptable salt thereof.
24. A compound according to any one of item 15 to 23 for the use as a medicament.
25. A compound according to any one of item 15 to 24 for use in the prophylaxis and/or treatment of diseases, disorders, therapeutic indications or medical conditions amenable for treatment with DHODH inhibitors.
26. A compound for use according to item 25 wherein the disease, disorder, therapeutic indication or medical condition is selected from the group comprising rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation or arthropathy.
27. A compound for use according to item 26 wherein the disease, disorder or therapeutic indication is selected from the group comprising graft versus host and host versus graft reactions, rheumatoid arthritis, multiple sclerosis, amyotrophic lateral sclerosis, lupus erythematosus, inflammatory bowel disease, cancer, COVID-19, influenza, ulcerative colitis, Crohn's disease, primary sclerosing cholangitis and psoriasis.
28. A pharmaceutical composition comprising a compound according to any one of item 15 to 23 and a pharmaceutically acceptable carrier or excipient.
29. A pharmaceutical composition of item 28, further comprising one or more additional therapeutic agents selected from antiviral agents, anti-inflammatory agents, immunosuppressive and/or immunomodulatory agents, steroids, non-steroidal anti-inflammatory agents, antihistamines, analgesics and suitable mixtures thereof.
30. A pharmaceutical composition of item 29, further comprising the additional therapeutic agent molnupiravir.

In particular embodiments, as used herein, wherein when any one of R¹ and R² is deuterium, the level of deuterium incorporation at each of R¹ and R² designated as deuterium is at least 52.5%, at least 75%, at least 82.5%, at least 90%, at least 95%, at least 97%, at least 98%, or at least 99%; when any one of R³, R⁴, R⁵ and R⁶ is deuterium, the level of deuterium incorporation at each of R³, R⁴, R⁵ and R⁶ designated as deuterium is at least 52.5%, at least 75%, at least 82.5%, at least 90%, at least 95%, at least 97%, at least 98%, or at least 99%; when any one of R⁷, R⁸, R⁹ and R¹⁰ is deuterium, the level of deuterium incorporation at each of R⁷, R⁸, R⁹ and R¹⁰ is at least 52.5%, at least 75%, at least 82.5%, at least 90%, at least 95%, at least 97%, at least 98%, or at least 99%; when any residue in R¹¹ contains one or more deuterium, the level of deuterium incorporation at each position in R¹¹ is at least 52.5%, at least 75%, at least 82.5%, at least 90%, at least 95%, at least 97%, at least 98%, or at least 99%; when any residue in ring A contains one or more deuterium, the level of deuterium incorporation at each position in ring A is at least 52.5%, at least 75%, at least 82.5%, at least 90%, at least 95%, at least 97%, at least 98%, or at least 99%; when any one of R²¹, R²², R²³, R²⁴, R²⁵ and R²⁶ is deuterium, the level of deuterium incorporation at each of R²¹, R²², R²³, R²⁴, R²¹ and R²⁶ is at least 52.5%, at least 75%, at least 82.5%, at least 90%, at least 95%, at least 97%, at least 98%, or at least 99%.

Quantitative analysis of specifically deuterated compounds can be achieved by a number of conventional methods, such as mass spectroscopy (peak area) or by quantifying the remaining residual ¹H-NMR signals of the specific deuteration site compared to signals from internal standards or other, non-deuterated ¹H signals in the compound.

In particular embodiments, the level of deuterium incorporation at each substituent designated as deuterium is at least 52.5%. More particularly, the level of deuterium incorporation at each substituent designated as deuterium is at least 90%. Even more particularly, the level of deuterium incorporation at each substituent designated as deuterium is at least 95%. Most particularly, the level of deuterium incorporation at each substituent designated as deuterium is at least 98%.

In particular embodiments, as used herein, ring A denotes a 5-membered heteroaryl, cyclopentenyl and heterocyclopentenyl, having one or more hydrogen atoms optionally replaced by deuterium, said ring A is unsubstituted or substituted with 1 to 5 substituents independently selected from the group consisting of halogen, CN, NO₂, oxo, OH, C₁₋₄-alkyl, O—C₁₋₄-alkyl, fluoro-C₁₋₄-alkyl and O-fluoro-C₁₋₄-alkyl, CO₂H and SO₃H, having one or more hydrogen atoms in alkyl optionally replaced by deuterium. More particularly, ring A denotes an unsubstituted 5-membered heteroaryl, cyclopentenyl and heterocyclopentenyl, having one or more hydrogen atoms optionally replaced by deuterium. More particularly,
ring

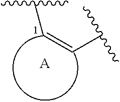

denotes to

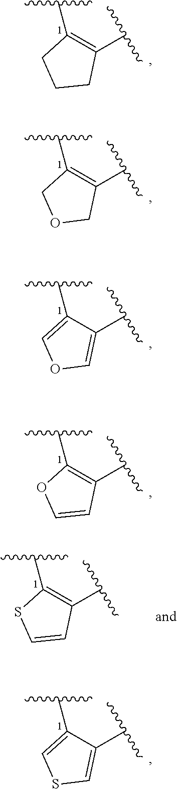

having one or more hydrogen atoms optionally replaced by deuterium.

More particularly, ring A denotes to

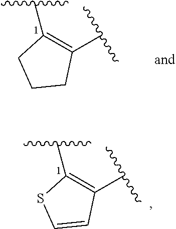 [SMILES: *C1=C(*)CCC1 ; *c1ccsc1*]

having one or more hydrogen atoms optionally replaced by deuterium. Even more particularly, ring A denotes to

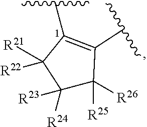

wherein R²¹, R²², R²³, R²⁴, R²⁵ and R²⁶ are independently selected from H and D. Most particularly, ring A denotes to

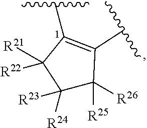

wherein R²¹, R²², R²³, R²⁴, R²⁵ and R²⁶ are H.

In particular embodiments, as used herein, ring A denotes a 5-membered heteroaryl, cyclopentenyl and heterocyclopentenyl, having one or more hydrogen atoms optionally replaced by deuterium, said ring A is unsubstituted or substituted with 1 to 5 substituents independently selected from the group consisting of halogen, CN, NO₂, oxo, OH, C₁₋₄-alkyl, O—C₁₋₄-alkyl, fluoro-C₁₋₄-alkyl and O-fluoro-C₁₋₄-alkyl, CO₂H and SO₃H, having one or more hydrogen atoms in alkyl optionally replaced by deuterium. More particularly, ring A denotes an unsubstituted 5-membered heteroaryl, cyclopentenyl and heterocyclopentenyl, having one or more hydrogen atoms optionally replaced by fluoro or deuterium.

More particularly,
ring

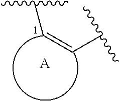

denotes to

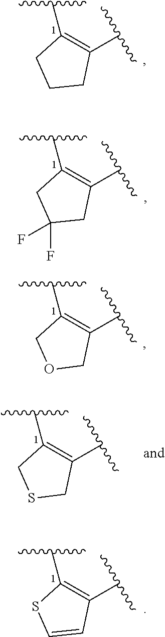

Most particularly, ring

A denotes to

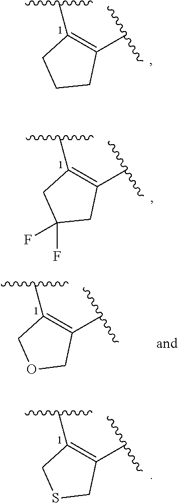

In particular embodiments of the present invention, R¹ is independently selected from H and D or a prodrug of the acid moiety. More particularly, R¹ is H.

In particular embodiments of the present invention, R² is selected from H and C₁₋₆-alkyl, said alkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S, and wherein R² or its substituents having one or more hydrogen atoms optionally replaced by deuterium. More particularly, R² is H, D or methyl.

In particular embodiments of the present invention, R² is independently selected from H and D. More particularly, R² is H.

In particular embodiments of the present invention, R¹ is H and R² is H.

In particular embodiments of the present invention, R³ is selected from H, D, halogen, CN, C₁₋₄-alkyl, O—C₁₋₄-alkyl, fluoro-C₁₋₄-alkyl and O-fluoro-C₁₋₄-alkyl, having one or more hydrogen atoms in alkyl optionally replaced by deuterium. More particularly, R³ is selected from H, D, F, Cl, CH₃, CHF₂, CF₃, CD₃, OCH₃, OCD₃, OCHF₂ and OCF₃. More particularly, R³ is selected from H, D, F and Cl. Most particularly, R³ is F.

In particular embodiments of the present invention, R⁴ is selected from H, D, halogen, CN, C₁₋₄-alkyl, O—C₁₋₄-alkyl, fluoro-C₁₋₄-alkyl and O-fluoro-C₁₋₄-alkyl, having one or more hydrogen atoms in alkyl optionally replaced by deuterium. More particularly, R⁴ is selected from H, D, F, Cl, CH₃, CHF₂, CF₃, CD₃, OCH₃, OCD₃, OCHF₂ and OCF₃. More particularly, R⁴ is selected from H, D and F. Most particularly, R⁴ is H.

In particular embodiments of the present invention, R⁵ is selected from H, D, halogen, CN, C₁₋₄-alkyl, O—C₁₋₄-alkyl, fluoro-C₁₋₄-alkyl and O-fluoro-C₁₋₄-alkyl, having one or more hydrogen atoms in alkyl optionally replaced by deuterium. More particularly, R⁵ is selected from H, D, F, Cl, CH₃, CHF₂, CF₃, CD₃, OCH₃, OCD₃, OCHF₂ and OCF₃. More particularly, R⁵ is selected from H, D and F. Most particularly, R⁵ is H.

In particular embodiments of the present invention, R⁶ is selected from H, D, halogen, CN, C₁₋₄-alkyl, O—C₁₋₄-alkyl, fluoro-C₁₋₄-alkyl and O-fluoro-C₁₋₄-alkyl, having one or more hydrogen atoms in alkyl optionally replaced by deuterium. More particularly, R⁶ is selected from H, D, F, Cl, CH₃, CHF₂, CF₃, CD₃, OCH₃, OCD₃, OCHF₂ and OCF₃. More particularly, R⁵ is selected from H, D and F. Most particularly, R⁶ is H.

In particular embodiments of the present invention,

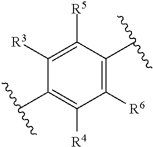

is selected from

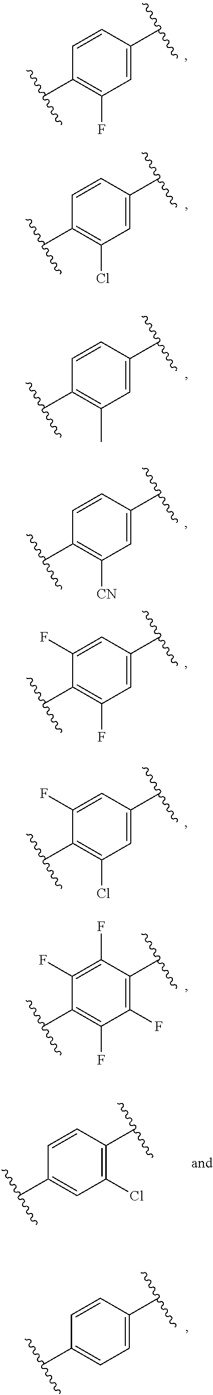

having one or more hydrogen atoms optionally replaced by deuterium. More particularly,

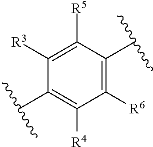

is selected from

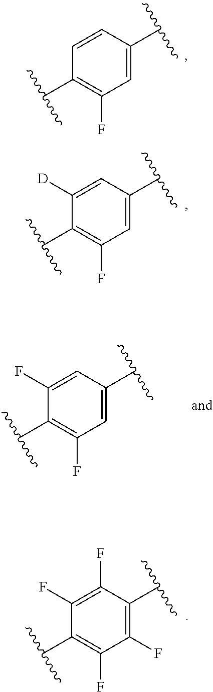 [SMILES: *c1ccc(*)c(F)c1 ; *c1cc([2H])c(*)c(F)c1 ; *c1cc(F)c(*)c(F)c1 ; *c1c(F)c(F)c(*)c(F)c1F]

Most particularly,

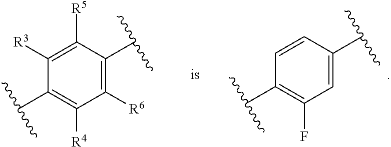

In particular embodiments of the present invention, ring B is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S, wherein said cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR²⁷, C₀₋₆-alkylene-(3- to 6-membered cycloalkyl), C₀₋₆-alkylene-(3- to 6-membered heterocycloalkyl), C₀₋₆-alkylene-S(═O)ₙ(═NR²⁹)ₘR²⁷, C₀₋₆-alkylene-NR²⁷S(═O)(═NR²⁹)_{y}R²⁷, C₀₋₆-alkylene-S(═O)(═NR²⁹)_{y}NR²⁷R²⁸, C₀₋₆-alkylene-NR²⁷ S(═O)ₓ(═NR²⁹)_{y}NR²⁷R²⁸, C₀₋₆-alkylene-CO₂R²⁷, C₀₋₆-alkylene-O—COR²⁷, C₀₋₆-alkylene-CONR²⁷R²⁸, C₀₋₆-alkylene-NR²⁷—COR²⁷, C₀₋₆-alkylene-NR²⁷—CONR²⁷R²⁸, C₀₋₆-alkylene-O—CONR²⁷R²⁸, C₀₋₆-alkylene-NR²⁷—CO₂R²⁷, C₀₋₆-alkylene-NR²⁷R²⁸, wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl;

and wherein optionally two adjacent substituents in the aryl or heteroaryl moiety form a 5- to 8-membered partially unsaturated cycle optionally containing 1 to 3 heteroatoms independently selected from O, S or N, wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
and wherein the residue —NR² on ring B is in a 1,4-orientation with respect to ring C, and ring B or its substituents having one or more hydrogen atoms optionally replaced by deuterium.

In more particular embodiments of the present invention, —NR²B is selected from

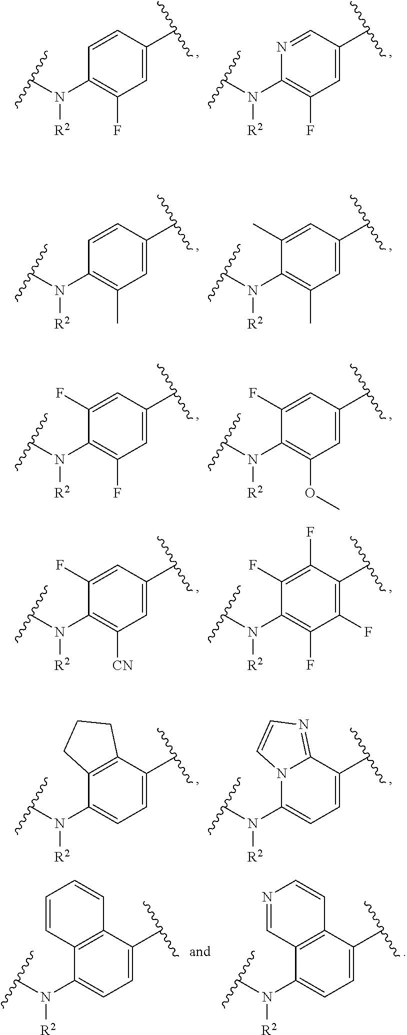

having one or more hydrogen atoms optionally replaced by deuterium.

In more particular embodiments of the present invention, —NR²B is selected from

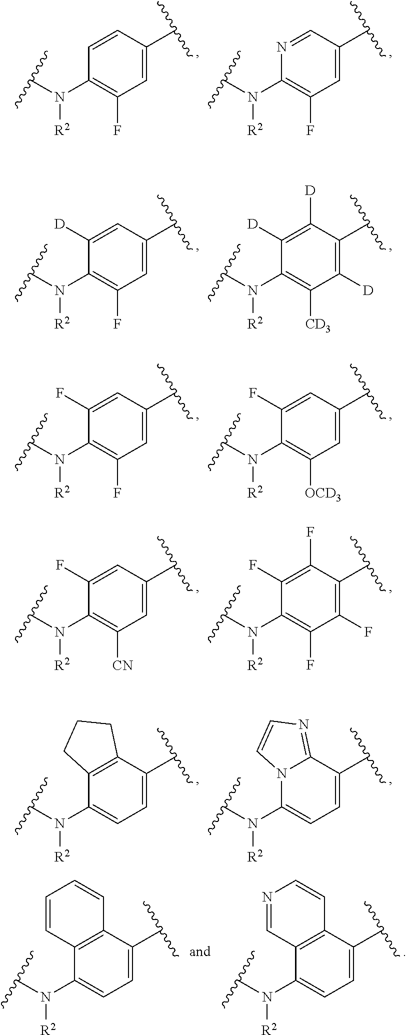

In even more particular embodiments of the present invention, —NR²B is selected from

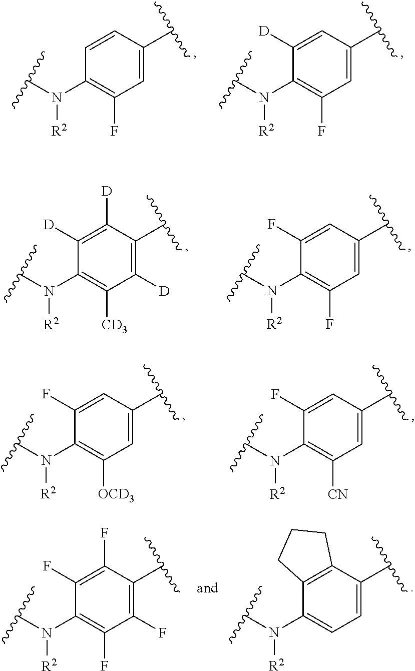

In most particular embodiments of the present invention, —NR²B is selected from

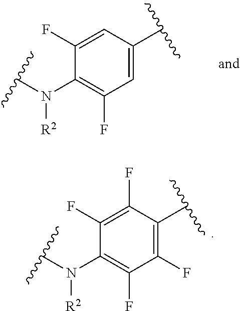

In particular embodiments of the present invention, R⁷ is selected from H, D, halogen, CN, C₁₋₄-alkyl, O—C₁₋₄-alkyl, fluoro-C₁₋₄-alkyl and O-fluoro-C₁₋₄-alkyl, having one or more hydrogen atoms in alkyl optionally replaced by deuterium. More particularly, R⁷ is selected from H and D. Most particularly, R⁷ is H.

In particular embodiments of the present invention, R⁸ is selected from H, D, halogen, CN, C₁₋₄-alkyl, O—C₁₋₄-alkyl, fluoro-C₁₋₄-alkyl and O-fluoro-C₁₋₄-alkyl, having one or more hydrogen atoms in alkyl optionally replaced by deuterium. More particularly, R⁸ is selected from H and D. Most particularly, R⁸ is H.

In particular embodiments of the present invention, R⁹ is selected from H, D, halogen, CN, C₁₋₄-alkyl, O—C₁₋₄-alkyl, fluoro-C₁₋₄-alkyl and O-fluoro-C₁₋₄-alkyl, having one or more hydrogen atoms in alkyl optionally replaced by deuterium. More particularly, R⁹ is selected from H and D. Most particularly, R⁹ is H.

In particular embodiments of the present invention, R¹⁰ is selected from H, D, halogen, CN, C₁₋₄-alkyl, O—C₁₋₄-alkyl, fluoro-C₁₋₄-alkyl and O-fluoro-C₁₋₄-alkyl, having one or more hydrogen atoms in alkyl optionally replaced by deuterium. More particularly, R¹⁰ is selected from H and D. Most particularly, R¹⁰ is H.

In particular embodiments of the present invention,

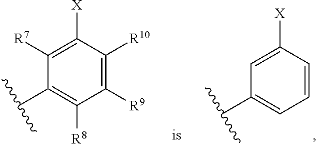

having one or more hydrogen atoms optionally replaced by deuterium. More particularly,

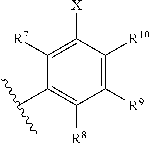

is selected from

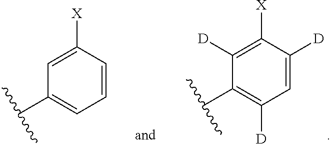

Most particularly,

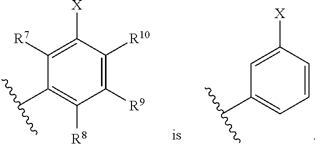

In particular embodiments of the present invention, X is selected from H, D, OH, OD, S(═O)_{y}R¹¹ and OR¹¹. More particularly, X is selected form OH and OR¹¹. Most particularly, X is OR¹¹.

In particular embodiments of the present invention, R¹¹ is selected from C₁₋₄-alkyl, C₃₋₄-cycloalkyl and fluoro-C₁₋₄-alkyl, having one or more hydrogen atoms in alkyl optionally replaced by deuterium. More particularly, R¹¹ is selected from CH₃, CD₃, CHF₂, CDF₂ and CF₃. Most particularly, R¹¹ is CD₃.

In particular embodiments of the present invention,

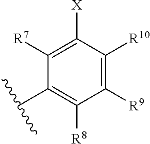

is selected from

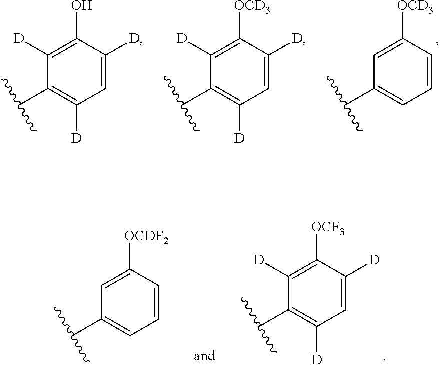

Most particularly,

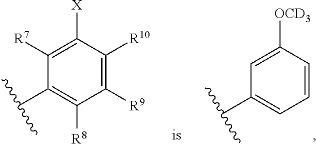

In particular embodiments of the present invention, ring C is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S, wherein said cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR³³)ₘR³¹, C₀₋₆-alkylene-NR³¹S(═O)ₓ(═NR³³)_{y}R³¹, C₀₋₆-alkylene-S(═O)ₓ(═NR³³)_{y}NR³¹R³², C₀₋₆-alkylene-NR³¹ S(═O)ₓ(═NR³³)_{y}NR³¹R³², C₀₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹, C₀₋₆-alkylene-NR³¹R³², wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl;

and wherein optionally two adjacent substituents in the aryl or heteroaryl moiety form a 5- to 8-membered partially unsaturated cycle optionally containing 1 to 3 heteroatoms independently selected from O, S or N, wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
and ring C or its substituents having one or more hydrogen atoms optionally replaced by deuterium;

In more particular embodiments of the present invention, ring C is phenyl, pyridyl or thiazolyl, wherein phenyl, pyridyl or thiazolyl is unsubstituted or substituted with 1 to 4 substituents independently selected from the group consisting of D and F; and X is selected from D, F, Cl, —CN, OH, C₁₋₄-alkyl, O—C₁₋₄-alkyl, fluoro-C₁₋₄-alkyl, O-fluoro-C₁₋₄-alkyl, having one or more hydrogen atoms optionally replaced by deuterium.

In more particular embodiments of the present invention, ring C is selected from

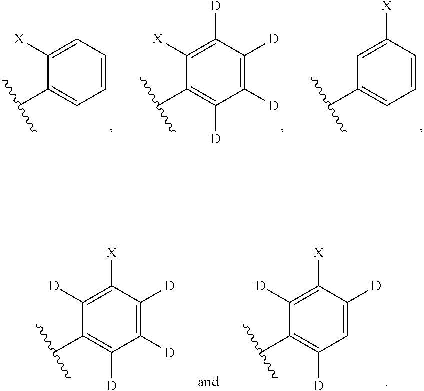

In more particular embodiments of the present invention, ring C is selected from

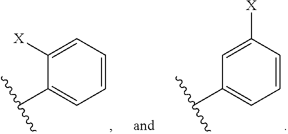

In more particular embodiments of the present invention, ring C is selected from

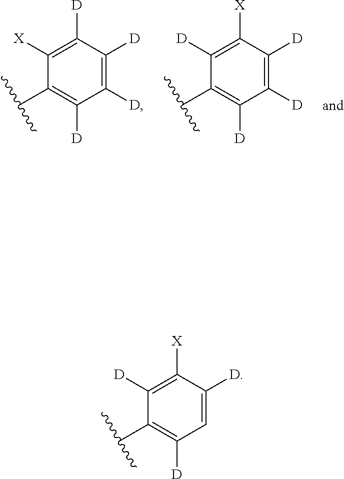

More particularly, ring C is

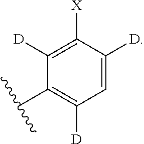

In particular embodiments of the present invention, X is selected from H, D, halogen, —CN, —NO₂, C₁₋₆-alkyl, —O—C₁₋₆-alkyl, O-halo-C₁₋₆-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR⁴³)ₘR⁴¹, C₀₋₆-alkylene-NR⁴¹S(═O)ₓ(═NR⁴³)_{y}R⁴¹, C₀₋₆-alkylene-S(═O)ₓ(═NR⁴³)_{y}NR⁴¹R⁴², C₀₋₆-alkylene-NR⁴¹S(═O)ₓ(═NR⁴³)_{y}NR⁴¹R⁴², C₀₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴² C₀₋₆-alkylene-NR⁴¹—CO₂R⁴¹, C₀₋₆-alkylene-NR⁴¹R⁴², wherein heterocycloalkyl comprises 1, 2, 3 or 4 heteroatoms independently selected from N, O, or S, wherein alkyl, alkylene, cycloalkyl and heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl,

and X or its substituents having one or more hydrogen atoms optionally replaced by deuterium.

In more particular embodiments of the present invention, X is selected from D, F, Cl, —CN, OH, C₁₋₄-alkyl, O—C₁₋₄-alkyl, fluoro-C₁₋₄-alkyl, O-fluoro-C₁₋₄-alkyl, having one or more hydrogen atoms optionally replaced by deuterium.

In more particular embodiments of the present invention, X is selected from D, F, OH, C₁₋₄-alkyl, O—C₁₋₄-alkyl, fluoro-C₁₋₄-alkyl, O-fluoro-C₁₋₄-alkyl, having one or more hydrogen atoms optionally replaced by deuterium.

In more particular embodiments of the present invention, X is selected from D, F, CD₃, CD₂CD₃, OH, OCD₃, OCD₂CD₃, O(CD₂)₃CD₃, OCF₃, OCDF₂ and OCHF₂. Most particularly, X is OCD₃.

In particular embodiments of the present invention, ring C is phenyl, pyridyl or thiazolyl, wherein phenyl, pyridyl or thiazolyl is unsubstituted or substituted with 1 to 4 substituents independently selected from the group consisting of D and F; and X is selected from D, F, Cl, —CN, OH, C₁₋₄-alkyl, O—C₁₋₄-alkyl, fluoro-C₁₋₄-alkyl, O-fluoro-C₁₋₄-alkyl, having one or more hydrogen atoms optionally replaced by deuterium.

In particular embodiments of the present invention,

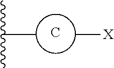

is selected from

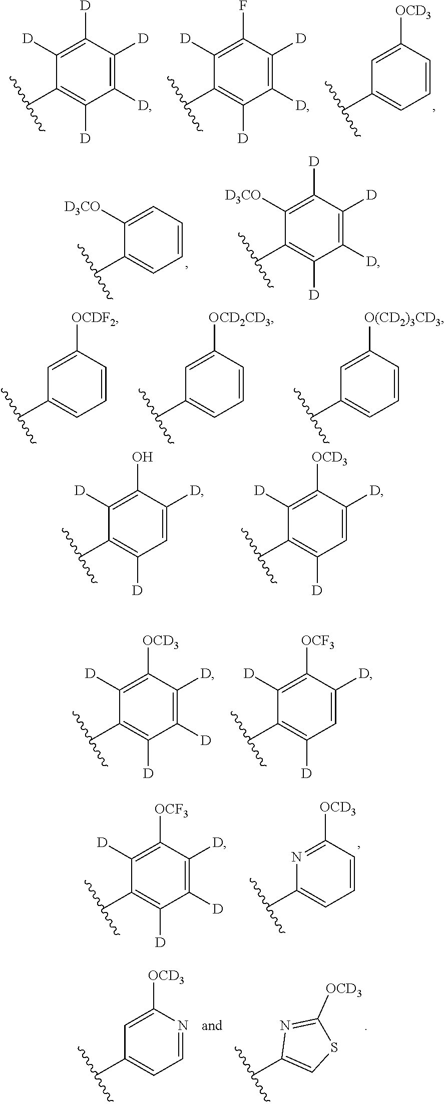

Most particularly,

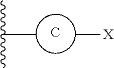

is selected from

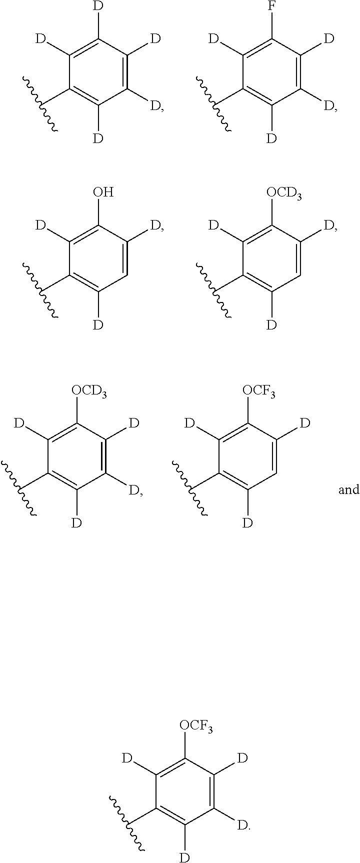

particularly,

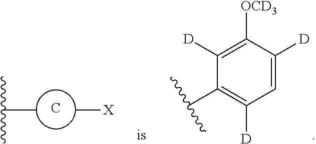

In particular embodiments of the present invention,

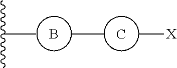

is selected from

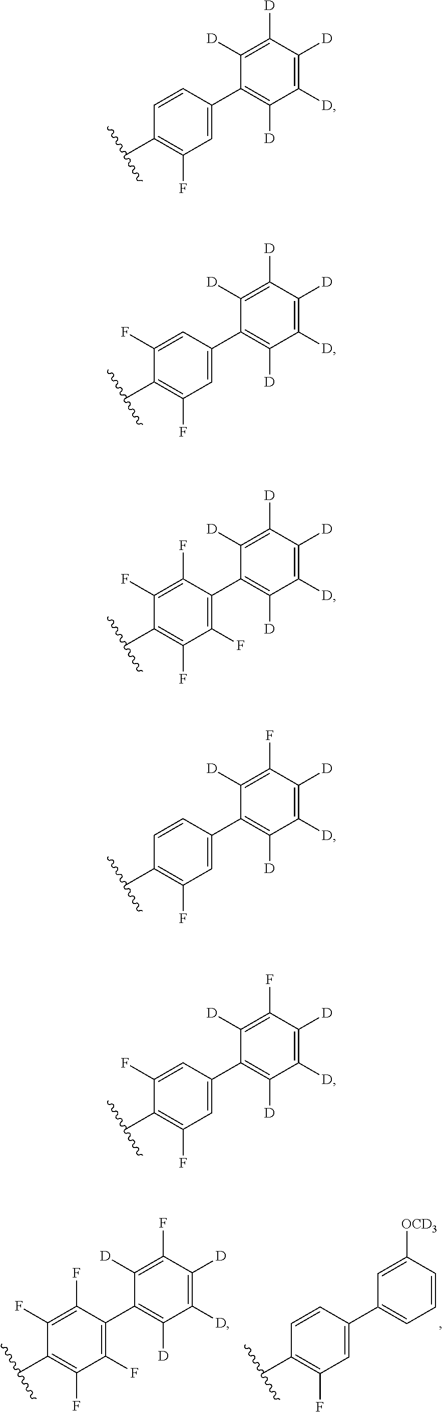

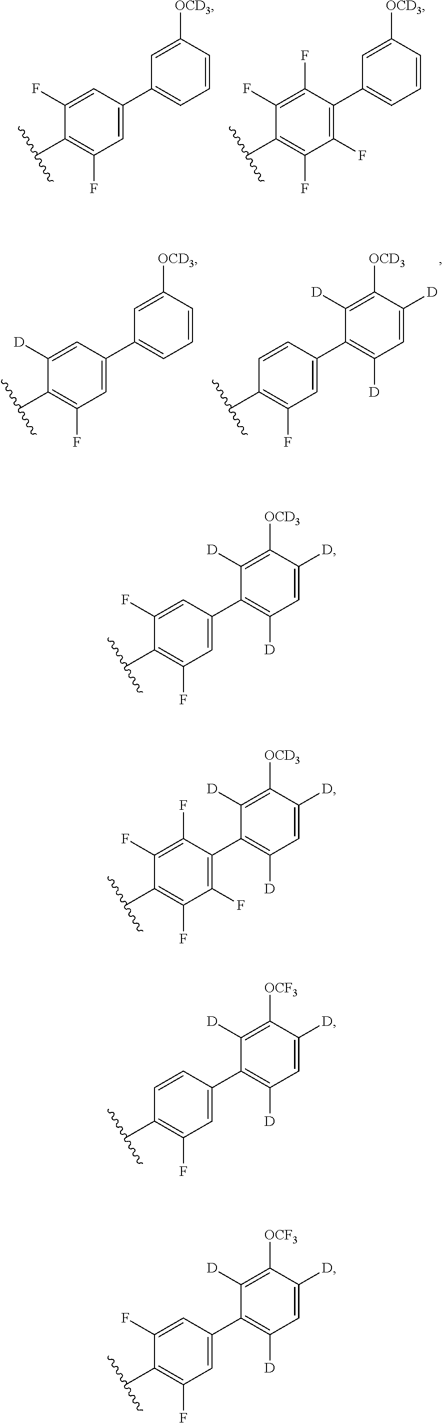

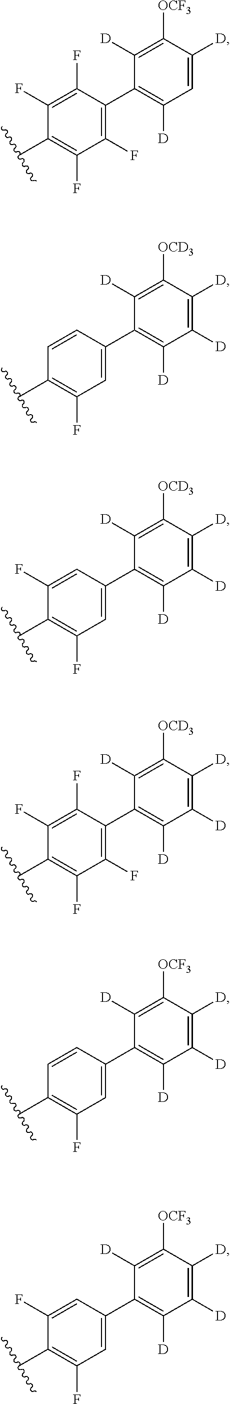

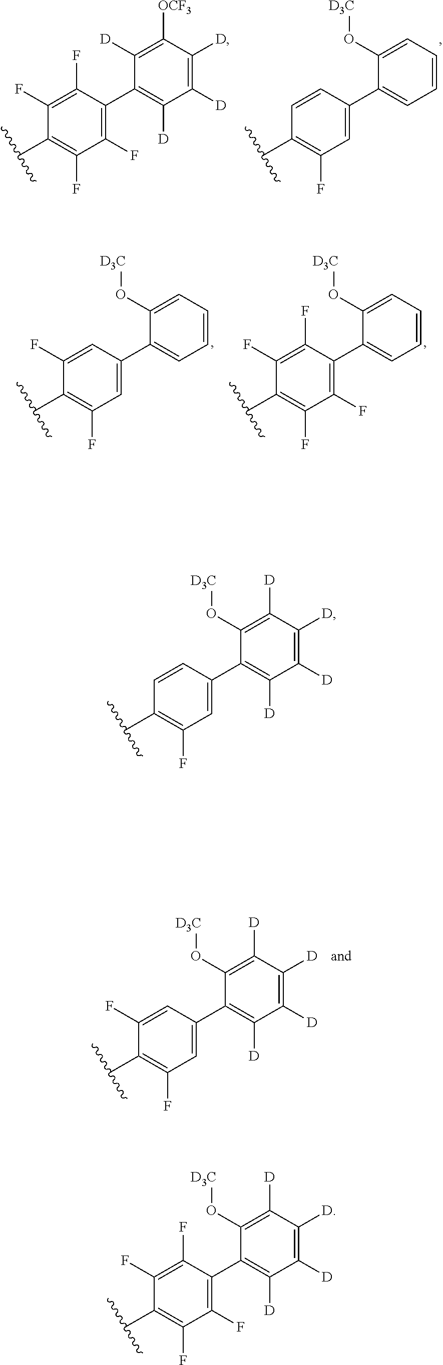

In more particular embodiments of the present invention,

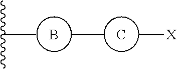

is selected from

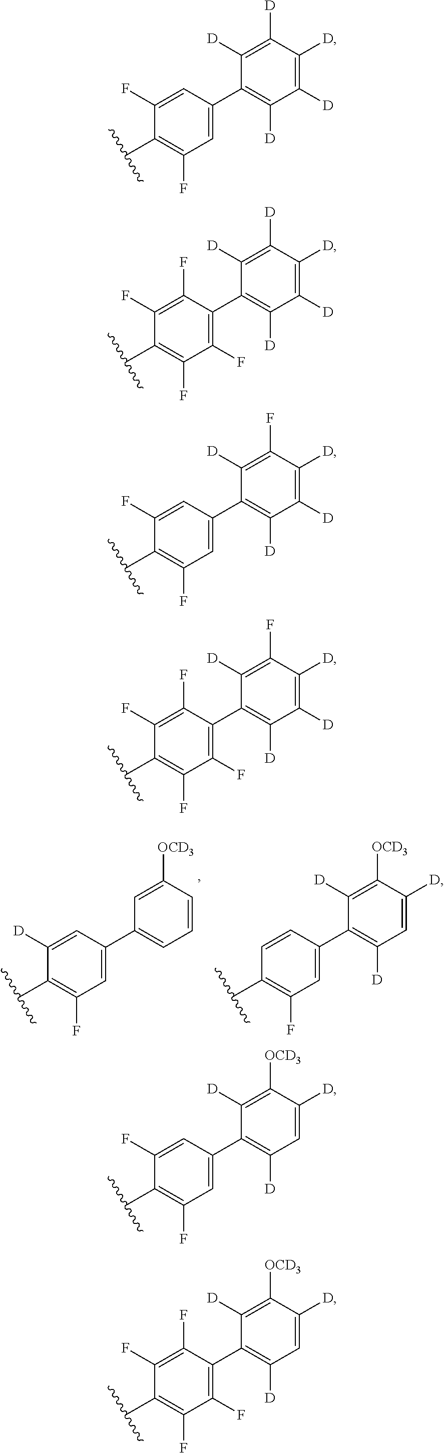

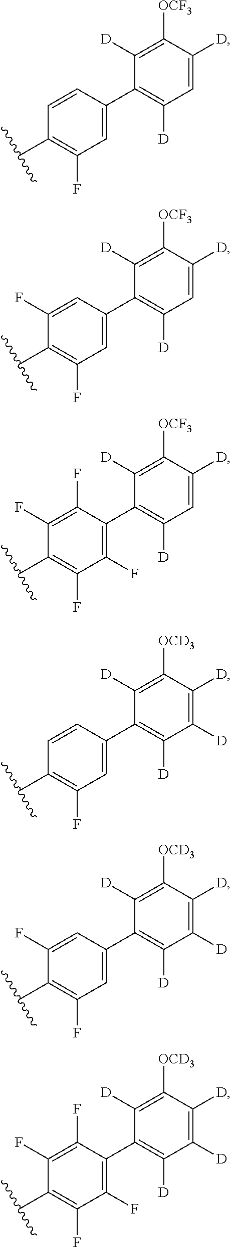

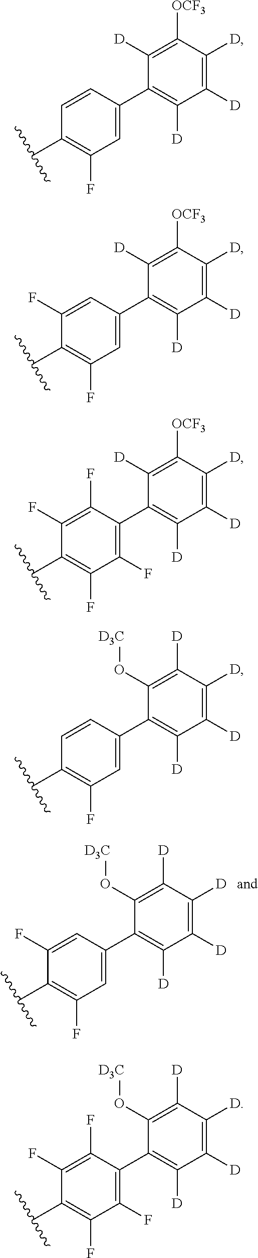

More particularly,

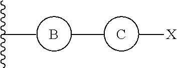

is selected from

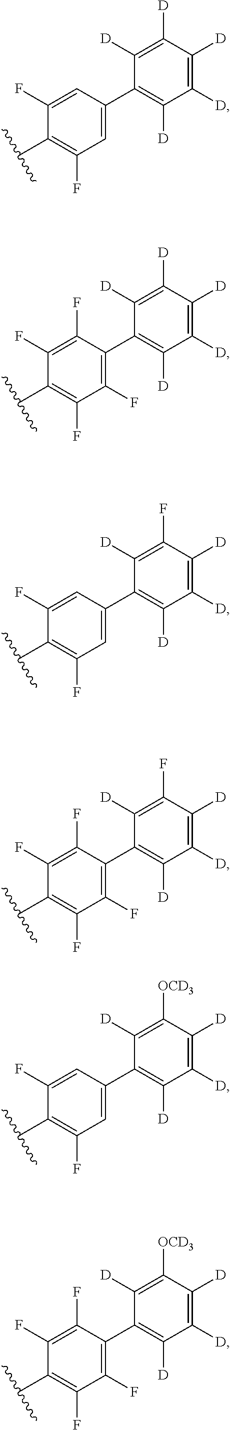

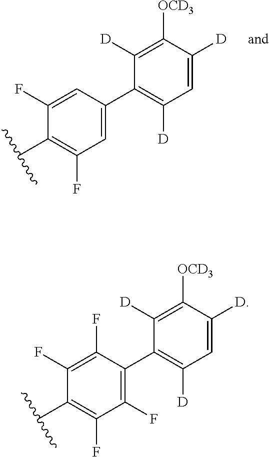

More particularly,

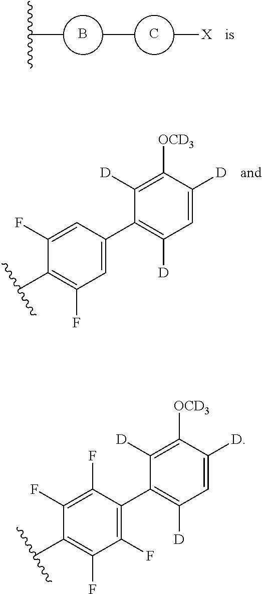

In particular embodiments of the present invention, R²⁷, R²⁸, R³¹, R³², R⁴¹, R⁴² are independently selected from H, C₁₋₆-alkyl, 3- to 6-membered cycloalkyl or 3- to 6-membered heterocycloalkyl, wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S; and R²⁷ and/or R²⁸ and/or R³¹ and/or R³² and/or R⁴¹ and/or R⁴² or its substituents, respectively, having one or more hydrogen atoms optionally replaced by deuterium;

or R²⁷ and R²⁸, R³¹ and R³², R⁴¹ and R⁴², respectively, when taken together with the nitrogen to which they are attached complete a 3- to 6-membered cycle containing carbon atoms and optionally containing 1 or 2 heteroatoms selected from O, S or N; and wherein this cycle is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl; R²⁷ and/or R²⁸ and/or R³¹ and/or R³² and/or R⁴¹ and/or R⁴² or its substituents, respectively, having one or more hydrogen atoms optionally replaced by deuterium.

In particular embodiments of the present invention, R²⁷, R²⁸, R³¹, R³², R⁴¹, R⁴² are independently selected from H, CH₃ and CD₃.

In particular embodiments of the present invention, R²⁹, R³³, R⁴³ are independently selected from H, —CN, —NO₂, C₁₋₆-alkyl, —CO—O—C₁₋₆-alkyl, 3- to 6-membered cycloalkyl or 3- to 6-membered heterocycloalkyl, wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S; R²⁹ and/or R³³ and/or R⁴³ or its substituents, respectively, having one or more hydrogen atoms optionally replaced by deuterium; In particular embodiments of the present invention, R²⁹, R³³, R⁴³ are independently selected from H, CH₃ and CD₃.

In particular embodiments of the present invention, n, m, x, y are independently selected from 0 to 2; with the proviso that the sum of integer m and n for the residue linked to the same sulfur atom is independently selected from 0 to 2; and with the proviso that the sum of integer x and y for the residue linked to the same sulfur atom is independently selected from 1 or 2.

In particular embodiments of the present invention, at least one hydrogen in ring A, ring B, ring C, R², R²⁷, R²⁸, R²⁹, R³¹, R³², R³³, R⁴¹, R⁴², R⁴³ and/or X is replaced by deuterium.

In particular embodiments of the present invention, at least one hydrogen in ring C and X is replaced by deuterium. More particularly, at least three hydrogen in ring C and X is replaced by deuterium. Most particularly, at least four hydrogen in ring C and X is replaced by deuterium.

In particular embodiments of the present invention, R¹ is H and R² is H;

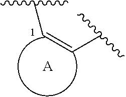

is selected from

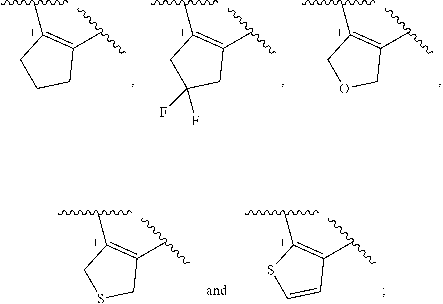 [SMILES: *C1=C(*)CCC1 ; *C1=C(*)CC(F)(F)C1 ; *C1=C(*)COC1 ; *C1=C(*)CSC1 ; *c1ccsc1*]

—NR²B is selected from

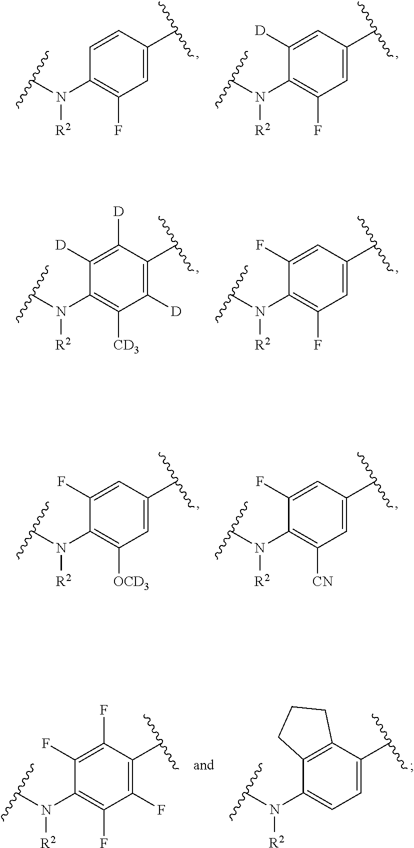

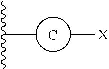

is selected from

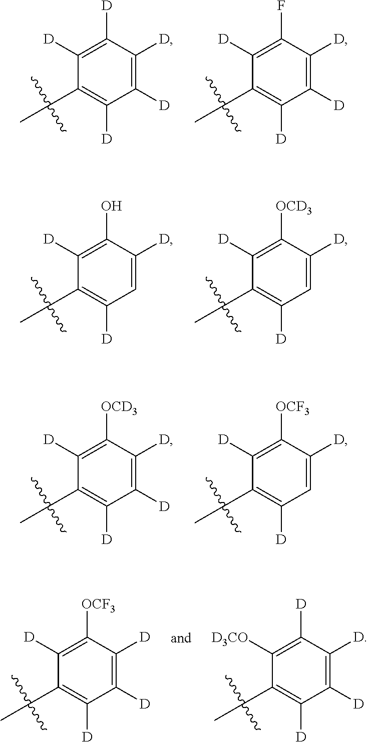

In more particular embodiments of the present invention, R¹ is H and R² is H;

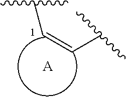

is selected from

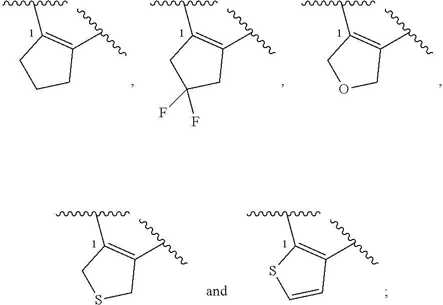 [SMILES: *C1=C(*)CCC1 ; *C1=C(*)CC(F)(F)C1 ; *C1=C(*)COC1 ; *C1=C(*)CSC1 ; *c1ccsc1*]

—NR²B is selected from

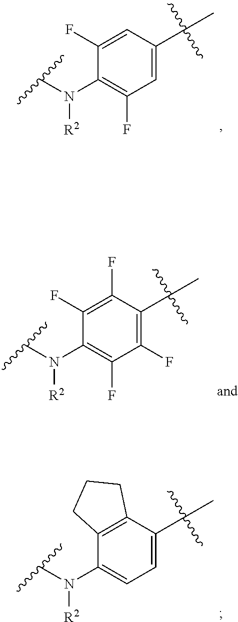

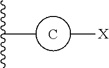

is selected from

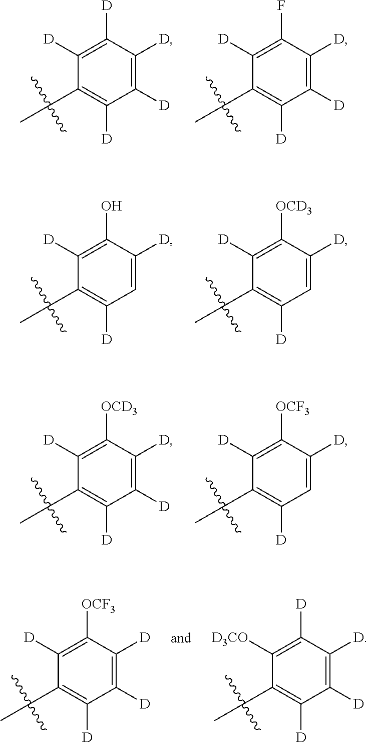

In most particular embodiments of the present invention, R¹ is H and R² is H;

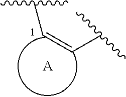

is selected from

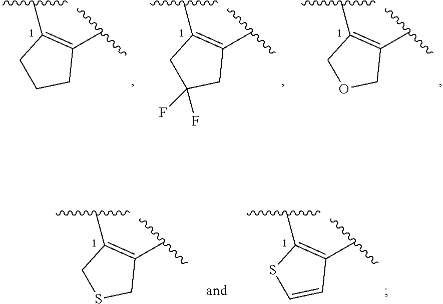 [SMILES: *C1=C(*)CCC1 ; *C1=C(*)CC(F)(F)C1 ; *C1=C(*)COC1 ; *C1=C(*)CSC1 ; *c1ccsc1*]

—NR²B is selected from

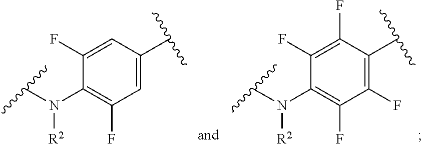

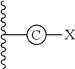

is selected from

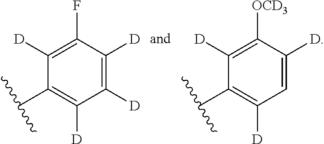

Particular compounds of the present invention are the compounds of the below examples of the present invention, more particularly the compounds of below examples 1, 2 and 6.

Particular compounds of the present invention are selected from

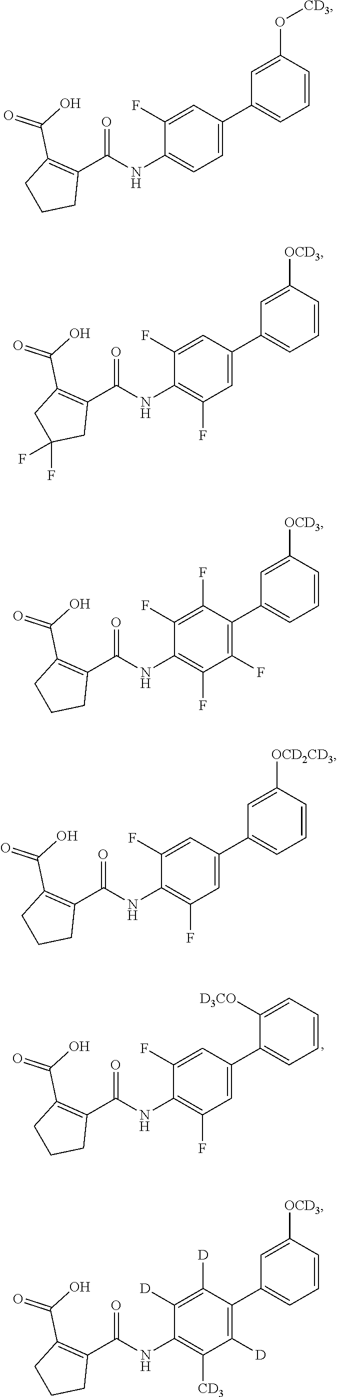

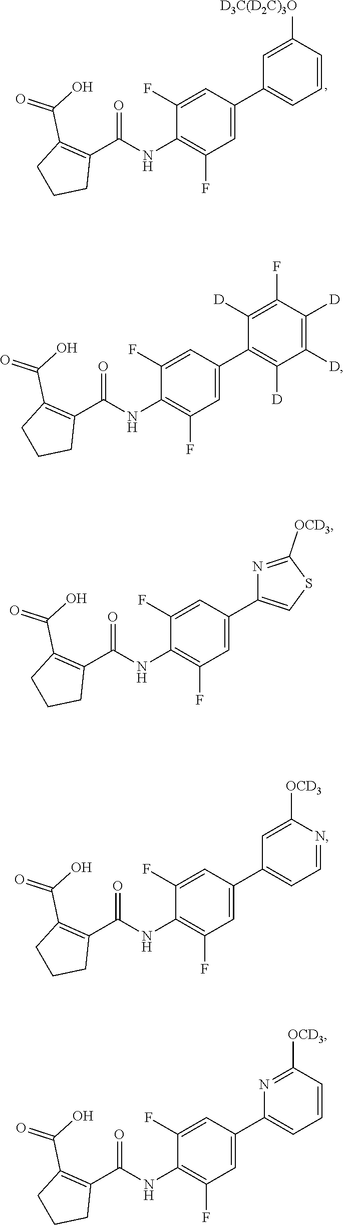

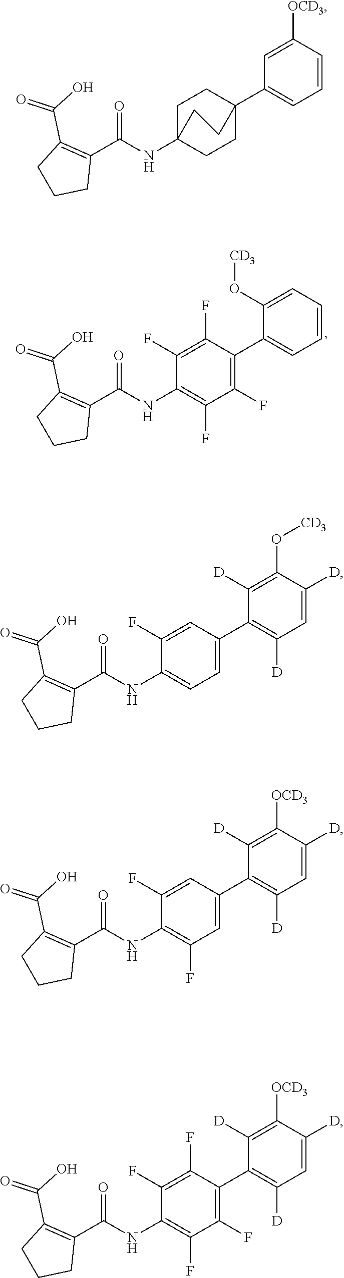

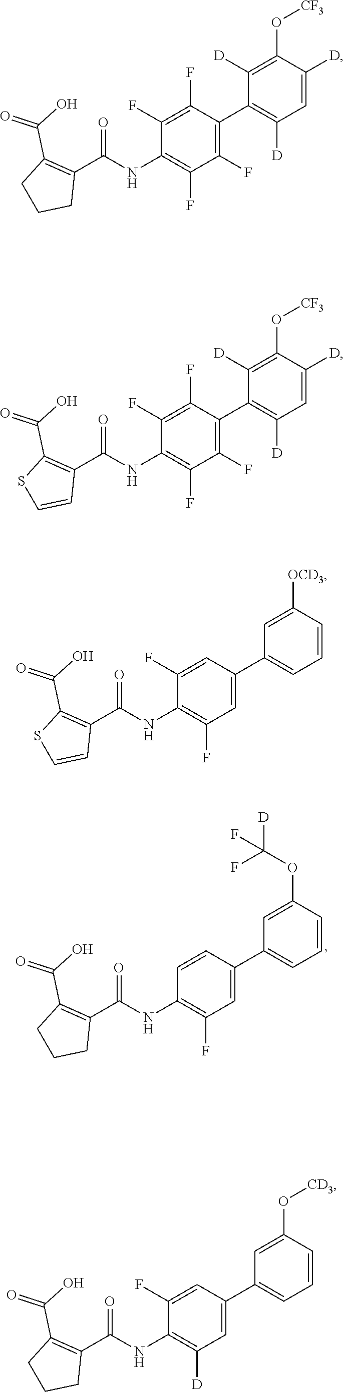

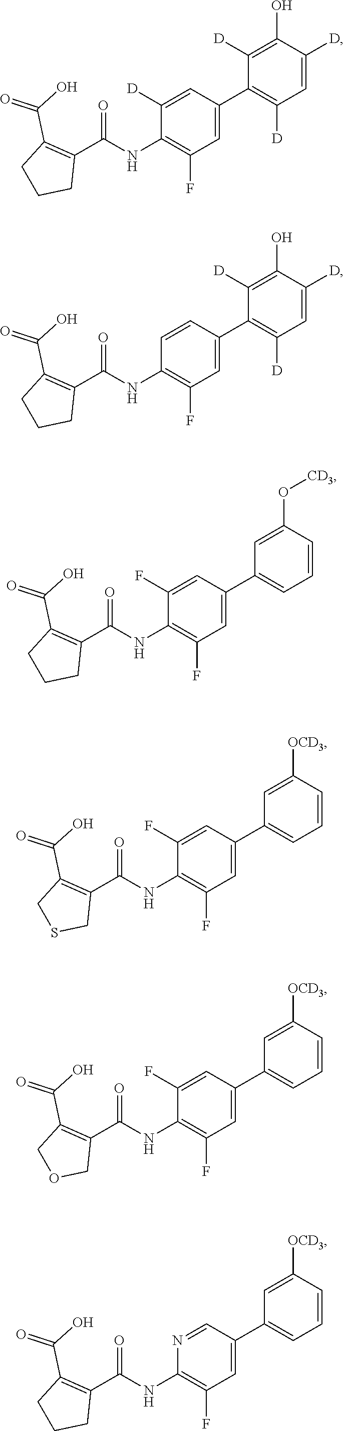

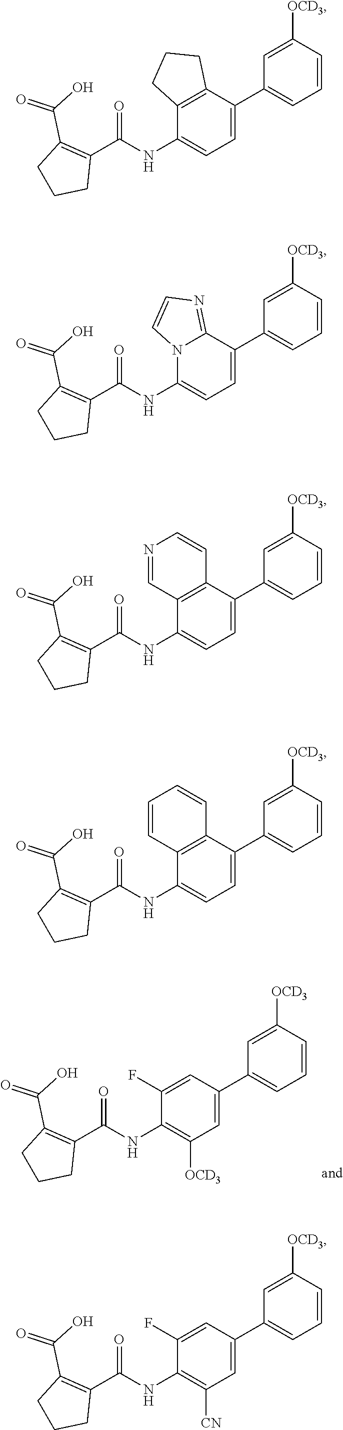

or a solvate or pharmaceutically acceptable salt thereof.

Particular compounds of the present invention are selected from

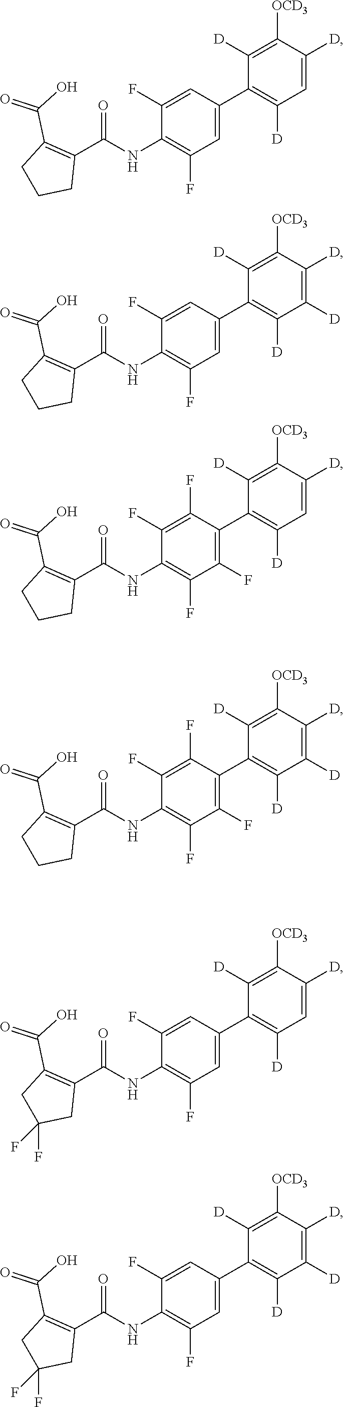

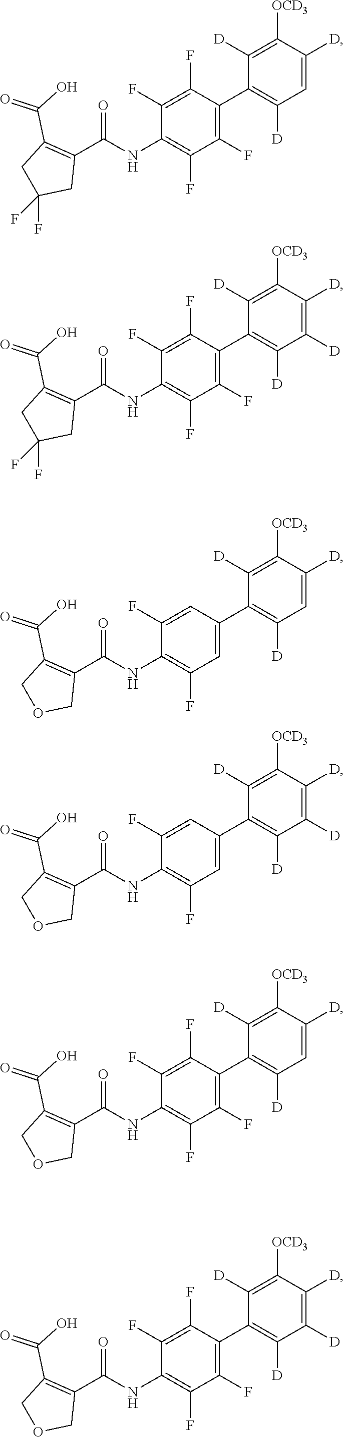

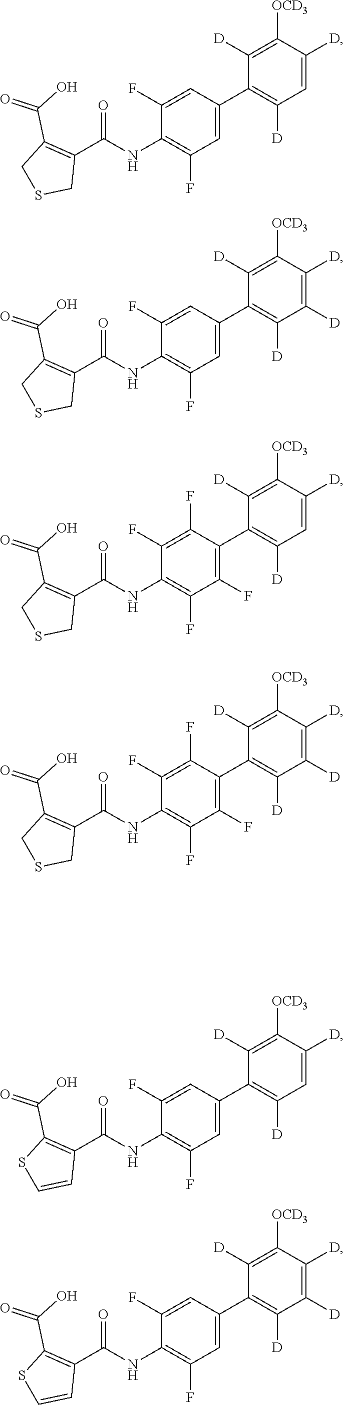

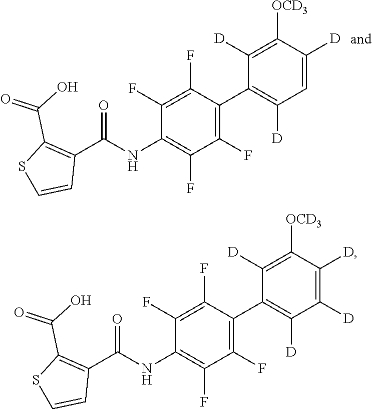

or a solvate or pharmaceutically acceptable salt thereof.

More particular compounds of the present invention are selected from

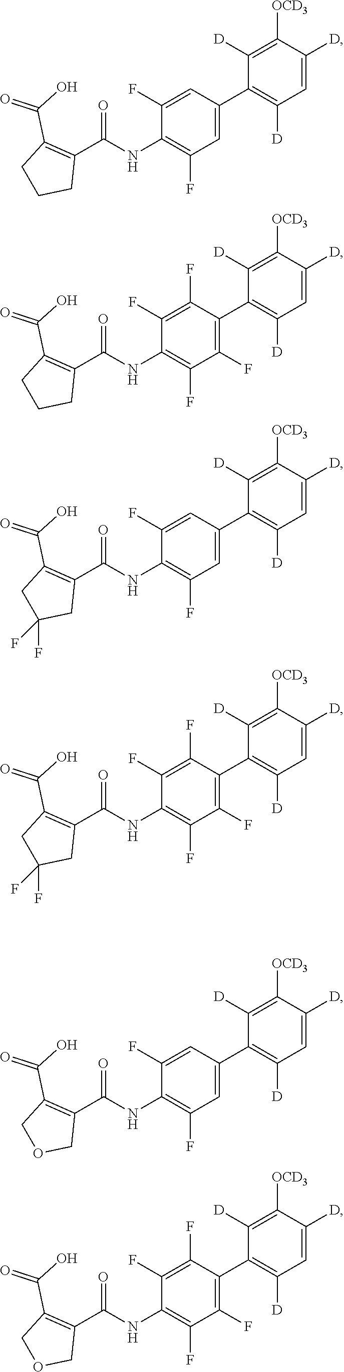

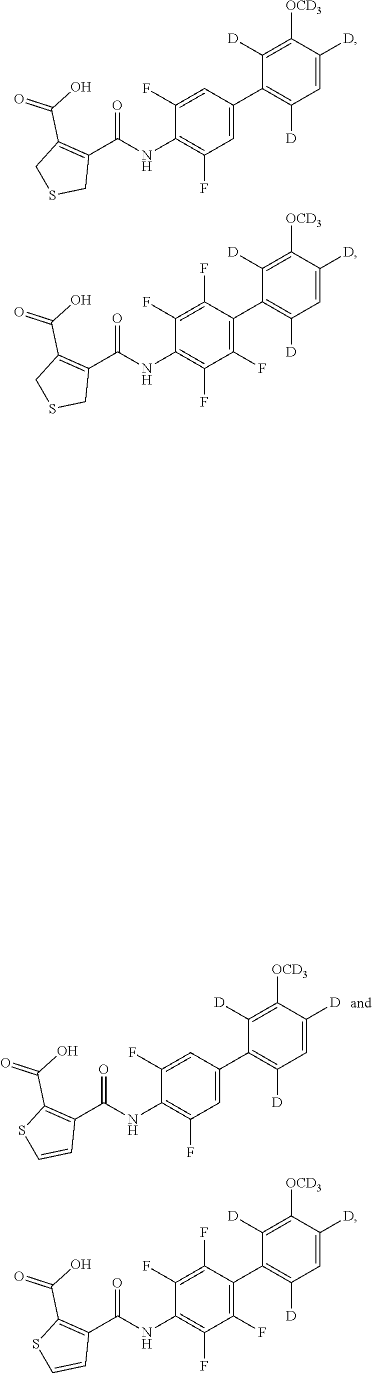

or a solvate or pharmaceutically acceptable salt thereof.

Most particular compounds of the present invention are selected from

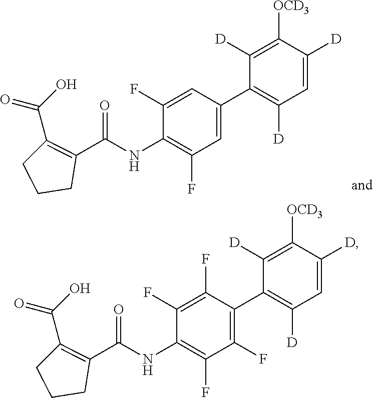

or a solvate or pharmaceutically acceptable salt thereof.

According to expert's knowledge the compounds of the invention as well as their salts may contain, e.g. when isolated in crystalline form, varying amounts of solvents. Included within the scope of the invention are therefore all solvates and in particular all hydrates of the compounds of Formula (I) as well as all solvates and in particular all hydrates of the salts of the compounds of Formula (I).

The present invention further relates to methods of prophylaxis and/or treatment of diseases, disorders, therapeutic indications or medical conditions which are described herein, particularly a disease or medical condition in which the inhibition of DHODH is beneficial, more particularly a disease or medical condition selected from the group comprising rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation or arthropathy, said method comprising administering to a subject in need thereof an effective amount of a compound of Formula (I) as described herein. Analogously, the present invention further relates to methods as the one described above, which encompass the further embodiments described herein, in particular the medical uses and compounds for use in medical treatments as described herein.

The present invention further relates to methods of prophylaxis and/or treatment of diseases, disorders, therapeutic indications or medical conditions which are described herein, particularly a disease or medical condition in which the inhibition of DHODH is beneficial, more particularly a disease or medical condition selected from graft versus host and host versus graft reactions, rheumatoid arthritis, multiple sclerosis, amyotrophic lateral sclerosis, lupus erythematosus, inflammatory bowel disease, cancer, COVID-19, ulcerative colitis, Crohn's disease, primary sclerosing cholangitis and psoriasis, said method comprising administering to a subject in need thereof an effective amount of a compound of Formula (I) as described herein.

The present invention further relates to pharmaceutical compositions, kits and kits-of parts comprising the compounds according to the present invention.

The present invention further relates to the use of the compounds according to the present invention for the production of pharmaceutical compositions which are employed for the treatment and/or prophylaxis of the diseases, disorders, illnesses and/or conditions as mentioned herein.

The present invention further relates to the methods and medical uses described herein, encompassing the pharmaceutical compositions as described herein.

The pharmaceutical compositions as described herein comprise one or more of the compounds according to this invention and a pharmaceutically acceptable carrier or excipient.

The pharmaceutical compositions as described herein comprise one or more of the compounds according to this invention and a pharmaceutically acceptable carrier or excipient, further comprising one or more additional therapeutic agents selected from antiviral agents, anti-inflammatory agents, immunosuppressive and/or immunomodulatory agents, steroids, non-steroidal anti-inflammatory agents, antihistamines, analgesics and suitable mixtures thereof.

Additionally, the invention relates to an article of manufacture, which comprises packaging material and a pharmaceutical agent contained within said packaging material, wherein the pharmaceutical agent is therapeutically effective against the medical conditions as described herein, and wherein the packaging material comprises a label or package insert which indicates that the pharmaceutical agent is useful for preventing or treating said medical conditions, and wherein said pharmaceutical agent comprises one or more compounds of Formula (I) according to the invention. The packaging material, label and package insert otherwise parallel or resemble what is generally regarded as standard packaging material, labels and package inserts for pharmaceuticals having related utilities.

The pharmaceutical compositions according to this invention are prepared by processes which are known per se and familiar to the person skilled in the art. As pharmaceutical compositions, the compounds of the invention (=active compounds) are either employed as such, or particularly in combination with suitable pharmaceutical auxiliaries and/or excipients, e.g. in the form of tablets, coated tablets, capsules, caplets, suppositories, patches (e.g. as TTS), emulsions, suspensions, gels or solutions, the active compound content advantageously being between 0.1 and 95% and where, by the appropriate choice of the auxiliaries and/or excipients, a pharmaceutical administration form (e.g. a delayed release form or an enteric form) exactly suited to the active compound and/or to the desired onset of action can be achieved.

The person skilled in the art is familiar with auxiliaries, vehicles, excipients, diluents, carriers or adjuvants which are suitable for the desired pharmaceutical formulations, preparations or compositions on account of his/her expert knowledge. In addition to solvents, gel formers, ointment bases and other active compound excipients, for example antioxidants, dispersants, emulsifiers, preservatives, solubilizers, colorants, complexing agents or permeation promoters, can be used.

Depending upon the particular disease, to be treated or prevented, additional therapeutic active agents, which are normally administered to treat or prevent that disease, may optionally be coadministered with the compounds according to the present invention. As used herein, additional therapeutic agents that are normally administered to treat or prevent a particular disease are known as appropriate for the disease being treated.

In a further aspect of the present invention, the compounds according to this invention or the salts or solvates of said compounds of Formula (I) may be combined with standard therapeutic agents which are commonly used for the treatment of the medical conditions as described herein.

The person skilled in the art is aware on the base of his/her expert knowledge of the total daily dosage(s) and administration form(s) of the additional therapeutic agent(s) coadministered. Said total daily dosage(s) can vary within a wide range. In practicing the present invention and depending on the details, characteristics or purposes of their uses mentioned above, the compounds according to the present invention may be administered in combination therapy separately, sequentially, simultaneously or chronologically staggered (e.g. as combined unit dosage forms, as separate unit dosage forms or an adjacent discrete unit dosage forms, as fixed or nonfixed combinations, as kit-of-parts or as admixtures) with one or more standard therapeutics, in particular art-known chemotherapeutic or target specific anti-cancer agents, such as those mentioned above.

Thus, a further aspect of the present invention is a combination or pharmaceutical composition comprising a first active ingredient, which is a compound according to this invention or a pharmaceutically acceptable salt or solvate thereof, a second active ingredient, which is an art-known standard therapeutic for the medical conditions as described herein, and optionally a pharmacologically acceptable carrier, diluent and/or excipient for sequential, separate, simultaneous or chronologically staggered use in therapy in any order, e.g. to treat, prevent or ameliorate in a patient the medical conditions as described herein. In this context, the present invention further relates to a combination comprising a first active ingredient, which is at least one compound according to this invention, and a second active ingredient, which is at least one art-known standard therapeutic for the medical conditions as described herein, for separate, sequential, simultaneous or chronologically staggered use in therapy, such as e.g. in therapy of those diseases mentioned herein.

The term “combination” according to this invention may be present as a fixed combination, a non-fixed combination or a kit-of-parts. A “fixed combination” is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A “kit-of-parts” is defined as a combination wherein the said first active ingredient and the said second active ingredient are present in more than one unit. One example of a “kit-of-parts” is a combination wherein the said first active ingredient and the said second active ingredient are present separately. The components of the kit-of-parts may be administered separately, sequentially, simultaneously or chronologically staggered.

The first and second active ingredient of a combination or kit-of-parts according to this invention may be provided as separate formulations (i.e. independently of one another), which are subsequently brought together for simultaneous, sequential, separate or chronologically staggered use in combination therapy; or packaged and presented together as separate components of a combination pack for simultaneous, sequential, separate or chronologically staggered use in combination therapy. The type of pharmaceutical formulation of the first and second active ingredient of a combination or kit-of-parts according to this invention can be similar, i.e. both ingredients are formulated in separate tablets or capsules, or can be different, i.e. suited for different administration forms, such as e.g. one active ingredient is formulated as tablet or capsule and the other is formulated for e.g. intravenous administration. The amounts of the first and second active ingredients of the combinations, compositions or kits according to this invention may together comprise a therapeutically effective amount for the treatment, prophylaxis or amelioration of a medical condition as described herein

A further aspect of the present invention is a method for treating cotherapeutically the medical conditions as described herein, in a patient in need of such treatment comprising administering separately, sequentially, simultaneously, fixed or non-fixed a therapeutically effective and tolerable amount of one or more of the compounds according to the present invention and a therapeutically effective and tolerable amount of one or more art-known therapeutic agents for the medical conditions as described herein, to said patient.

References and claims to the use of a compound of the Formula (I) or a pharmaceutically acceptable salt or solvate thereof in the manufacture of a medicament for the treatment of a disease or medical condition in their general and specific forms likewise refer to the corresponding methods of treating said disease or medical condition, said method comprising administering a therapeutically effective and tolerable amount of a compound of the Formula (I) or a pharmaceutically acceptable salt or solvate thereof to a subject in need thereof, compositions comprising a compound of the Formula (I) or a pharmaceutically acceptable salt or solvate thereof for the treatment of said disease or medical condition, a compound of the Formula (I) or a pharmaceutically acceptable salt or solvate thereof for use in the treatment of said disease or medical condition, and vice versa.

For the production of the pharmaceutical compositions, the compounds of the invention (=active compounds) are particularly mixed with suitable pharmaceutical auxiliaries and further processed to give suitable pharmaceutical formulations. Suitable pharmaceutical formulations are, for example, powders, emulsions, suspensions, sprays, oils, ointments, fatty ointments, creams, pastes, gels or solutions. The pharmaceutical compositions according to the invention are prepared by processes known per se.

The dosage of the active compounds is carried out in the customary order of magnitude. Topical application forms (such as ointments) thus contain the active compounds in a concentration of, for example, 0.1 to 99%. The customary dose in the case of systemic therapy (p.o.) is usually between 0.3 and 30 mg/kg per day, (i.v.) is usually between 0.3 and 30 mg kg/h. The choice of the optimal dosage regime and duration of medication, particularly the optimal dose and manner of administration of the active compounds necessary in each case can be determined by a person skilled in the art on the basis of his/her expert knowledge.

The class of compounds of the present invention is useful for the development of medicaments suitable for the treatment of autoimmune or viral diseases and chronic inflammation or, more generally, for the treatment of diseases where the inhibition of DHODH is beneficial. The compounds of the present invention are also useful for the treatment of diseases such as rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation or arthropathy. More specifically, the disease is selected graft versus host and host versus graft reactions, rheumatoid arthritis, multiple sclerosis, amyotrophic lateral sclerosis, lupus erythematosus, inflammatory bowel disease, cancer, COVID-19, influenza, ulcerative colitis, Crohn's disease, primary sclerosing cholangitis and psoriasis.

The class of compounds of the present invention is useful for the treatment of viral diseases, especially acute viral infections selected from Coronavirus infections, COVID-19, SARS, flu/influenza (and avian influenza), HIV/Aids, chickenpox (Varicella), cytomegalovirus, Dengue Fever, German measles (Rubella), hand-foot-mouth disease, hantavirus infections, all forms of hepatitis, Lassa fever, Marburg virus infections, measles, meningitis, MERS-CoV, mumps, norovirus infections, herpes simplex virus infections, smallpox, rotavirus infections, Ebola virus, poliovirus infections, rhinovirus infections, parainflunenzavirus infections, RSV infections, HCMV infections and bannavirus infections. Most preferred as COVID-19, flu/influenza and rhinovirus infections, most preferred is COVID-19. It is understood, that also mutated forms of the virus (e.g. of SARS-CoV-2) are covered.

Combination or Alternation Therapy

The compounds or their pharmaceutically acceptable salts as described herein can be administered on top of the current standard of care for patients, or in combination or alternation with any other compound or therapy that the healthcare provider deems beneficial for the patient. The combination and/or alternation therapy can be therapeutic, adjunctive or palliative.

Especially preferred is a combination or alternation therapy for the treatment of anti-viral infections, especially Covid-19:

It has been observed that high levels of the cytokine interleukin-6 (IL-6) are a precursor to respiratory failure and death in COVID-19 patients. To treat this surge of an immune response, which may constitute a cytokine storm, patients can be administered an IL-6-targeting monoclonal antibody, pharmaceutical inhibitor or protein degrader such as a bispecific compound that binds to IL-6 and also to a protein that mediates degradation. Examples of antibodies include tocilizumab, sarilumab, siltuximab, olokizumab and clazakizumab. In one embodiment, a compound of Formula (I) or a pharmaceutically acceptable salt thereof is administered in combination or in alternation with tocilizumab or sarilumab. Additional nonlimiting examples of immunosuppressant drugs used to treat the overreacting immune system include Janus kinase inhibitors (tofacitinib, baricitinib, filgotinib); calcineurin inhibitors (cyclosporine), tacrolimus, mTOR inhibitors (sirolimus, everolimus) and IMDH inhibitors (azathioprine). Additional antibodies and biologics include abatacept, adalimumab, anakinra, certolizumab, etanercept, golimumab, infliximab, ixekizumab, natalizumab, rituximab, secukinumab, tocilizumab, ustekinumab, vedolizumab, basiliximab and daclizumab.

IL-1 blocks the production of IL-6 and other proinflammatory cytokines. COVID patients are also sometimes treated with anti-IL-1 therapy to reduce a hyperinflammatory response, for example, an intravenous administration of anakinra. Anti-IL-1 therapy generally may be for example, a targeting monoclonal antibody, pharmaceutical inhibitor or protein degrader such as a bispecific compound that binds to IL-1 and also to a protein that mediates degradation.

Patients with COVID often develop viral pneumonia, which can lead to bacterial pneumonia. Patients with severe COVID-19 can also be affected by sepsis or “septic shock”. Treatment for bacterial pneumonia secondary to COVID or for sepsis includes the administration of antibiotics, for example a macrolide antibiotic, including azithromycin, clarithromycin, erythromycin, or roxithromycin. Additional antibiotics include amoxicillin, doxycycline, cephalexin, ciprofloxacin, clindamycin, metronidazole, sulfamethoxazole, trimethoprim, amoxicillin, clavulanate or levofloxacin. In one embodiment, thus a compound of Formula (I) or a pharmaceutically acceptable salt thereof is administered in combination or in alternation with an antibiotic, for example, azithromycin. Some of these antibiotics such as azithromycin have independent anti-inflammatory properties. Such drugs may be used both as anti-inflammatory agents for COVID patients and have a treatment effect on secondary bacterial infections.

A unique challenge in treating patients infected with COVID-19 is the relatively long-term need for sedation if patients require mechanical ventilation which might last up to or greater than 5, 10 or even 14 days. For ongoing pain during this treatment, analgesics can be added sequentially and for ongoing anxiety, sedatives can be added sequentially. Non-limiting examples of analgesics include acetaminophen, ketamine and PRN opioids (hydromorphone, fentanyl, and morphine). Non-limiting examples of sedatives include melatonin, atypical antipsychotics with sedative-predominant properties (olanzapine, quetiapine), propofol or dexmedetomidine, haloperidol and phenobarbital. In one embodiment, a compound of Formula (I) or a pharmaceutically acceptable salt, a solvate, a solvate of a salt, a hydrate or a polymorph thereof is administered in combination or in alternation with a pain reliever, such as acetaminophen, ketamine, hydromorphone, fentanyl, or morphine. In one embodiment, a compound of Formula (I) a pharmaceutically acceptable salt, a solvate, a solvate of a salt, a hydrate or a polymorph thereof is administered in combination or in alternation with a sedative, such as melatonin, olanzapine, quetiapine, propofol, dexmedetomidine, haloperidol or phenobarbital.

In one embodiment, a compound of the present invention is used in an effective amount in combination with a protease inhibitor such as PF-07304814, PF-00835231, PF-07321332 (nirmatrelvir), lopinavir or ritonavir. In one more special embodiment, protease inhibitor is PF-07321332 (nirmatrelvir).

In one embodiment, a compound of the present invention is used in an effective amount in combination with a RNA replication modulator such as N4-hydroxycytidine or a prodrug thereof may also be administered. In one special embodiment, the RNA replication modulator is a N4-hydroxycytidine prodrug as described in WO 2019/113462. In one more special embodiment, the RNA replication modulator is molnupiravir.

In one embodiment, a compound of the present invention is used in an effective amount in combination with halofuginol or an enantiomer, tautomer, solvate or pharmaceutically acceptable salt thereof.

In one embodiment, a compound of the present invention is used in an effective amount in combination with dipyridamole or a solvate or pharmaceutically acceptable salt thereof.

In one embodiment, a compound of the present invention is used in an effective amount in combination with gemcitabine or a solvate or pharmaceutically acceptable salt thereof.

In one embodiment, a compound of the present invention is used in an effective amount in combination with ΔT-527 (RO7496998) or a solvate or pharmaceutically acceptable salt thereof.

Additional drugs that may be used in the treatment of a COVID patient include, but are not limited to aspirin, colchicine, dimethyl fumarate, acalabrutinib, favipiravir, fingolimod, methylprednisolone, bevacizumab, tocilizumab, umifenovir, losartan and the monoclonal antibody combination of REGN3048 and REGN3051 or ribavirin. Any of these drugs or vaccines can be used in combination or alternation with an active compound provided herein to treat a viral infection susceptible to such.

In one embodiment, a compound of the present invention is used in an effective amount in combination with anti-coronavirus vaccine therapy, including but not limited to mRNA-1273 (Moderna), AZD-1222 (AstraZeneca and University of Oxford), BNT162b2 (BioNTech), CoronaVac (Sinovac), NVX-CoV 2372 (NovoVax), SCB-2019 (Sanofi and GSK), ZyCoV-D (Zydus Cadila) and CoVaxin (Bharat Biotech). In another embodiment, a compound of the present invention is used in an effective amount in combination with passive antibody therapy or convalescent plasma therapy.

SARS-CoV-2 is constantly mutating, which many increase virulence and transmission rates. Drug-resistant variants of viruses may emerge after prolonged treatment with an antiviral agent. Drug resistance may occur by mutation of a gene that encodes for an enzyme used in viral replication. The efficacy of a drug against an RNA virus infection in certain cases can be prolonged, augmented or restored by administering the compound in combination or alternation with another and perhaps even two or three other, antiviral compounds that induce a different mutation or act through a different pathway, from that of the principle drug. A variant of a known virus can refer to a virus carrying one or more nucleotide mutations in the viral genome as compared to the known virus, for instance at least 1, 2, 3, 4, 5, 6, 7, 8, 9, 10, 15, 20, 30, 40, 60, 100, 200, 300 or even more nucleotide mutations. Mutations can refer to nucleotide deletion, insertion, or substitution. In some cases, a variant can have at most 50%, 40%, 30%, 20%, 10%, 5%, 4%, 3%, 2% or 1% of the viral genome different than the genome of a known virus.

Alternatively, the pharmacokinetics, biodistribution, half-life or other parameter of the drug can be altered by such combination therapy (which may include alternation therapy if considered concerted).

Examples of other therapeutic agents that may be combined with a compound of Formula (I) or a pharmaceutically acceptable salt, a solvate, a solvate of a salt, a hydrate or a polymorph thereof, either administered separately, or in the same pharmaceutical composition include, but are not limited to a:
(1) Protease inhibitor;
(2) Polymerase inhibitor (e.g. gemcitabine);
(3) Allosteric polymerase inhibitor;
(4) Interferon alfa-2a, which may be pegylated or otherwise modified, and/or ribavirin;
(5) Non-substrate-based inhibitor;
(6) Helicase inhibitor;
(7) Primase-helicase inhibitor;
(8) Antisense oligodeoxynucleotide (S-ODN);
(9) Aptamer;
(10) Nuclease-resistant ribozyme;
(11) iRNA, including microRNA and SiRNA;
(12) Antibody, partial antibody or domain antibody to the virus;
(13) Viral antigen or partial antigen that induces a host antibody response;
(14) NOD-, LRR- and pyrin domain-containing protein 3 (NLRP3);
(15) Glutamyl-prolyl-tRNA synthetase inhibitor (e.g. halofuginone);
(16) Equilibrative nucleoside transporter (ENT) inhibitor (e.g. dipyridamole);
(17) other DHODH inhibitors (e.g. brequinar, teriflunomide, leflunomide, PTC299, MEDS433, ΔG-636, ASLAN003, JNJ-74856665, RP7214, PP-001 and BAY2402234).

It will be recognized that some variation of natural isotopic abundance occurs in a synthesized compound depending upon the origin of chemical materials used in the synthesis. Thus, a preparation of vidofludimus (and other compounds according to Formula (I) specifically substituted with a deuterium) will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Comp. Biochem. Physiol. 1998; 119A:725.

The term “isotopic enrichment factor” at a particular position normally occupied by hydrogen refers to the ratio between the abundance of deuterium at the position and the natural abundance of deuterium at that position. By way of example, an isotopic enrichment factor of 3500 means that the amount of deuterium at the particular position is 3500-fold the natural abundance of deuterium, or that 52.5% of the compounds have deuterium at the particular position (i.e., 52.5% deuterium incorporation at the given position). The abundance of deuterium in the oceans of Earth is approximately one atom in 6500 hydrogen atoms (about 154 parts per million (ppm)). Deuterium thus accounts for approximately 0.015 percent (on a weight basis, 0.030 percent) of all naturally occurring hydrogen atoms in the oceans on Earth; the abundance changes slightly from one kind of natural water to another.

When a particular position in a compound of the invention (e.g., a compound represented by Formula (I) or a pharmaceutically acceptable salt and/or solvate thereof) is designated by name or structure as containing hydrogen or deuterium, it is to be understood that the position can contain hydrogen at its natural abundance or can be enriched in deuterium with an isotopic enrichment factor of, for example, at least 835 (12.5% deuterium incorporation), of at least 1670 (25% deuterium incorporation, of at least 3500 (52.5% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

When a particular position in a compound of the invention (e.g., a compound represented by Formula (I) or a pharmaceutically acceptable salt and/or solvate thereof) is designated specifically by name or structure as “H” or “hydrogen”, the position is understood to have hydrogen at its natural abundance isotopic composition.

When a particular position in a compound of the invention (e.g., a compound represented by Formula (I) or a pharmaceutically acceptable salt and/or solvate thereof) is designated specifically by name or structure as “D” or “deuterium”, the position is understood to have deuterium at an abundance that is at least 3340 times of the natural abundance of deuterium, which is 0.015% (i.e., at least 50.1% incorporation of deuterium), at least 3500 times of the natural abundance of deuterium (52.5% deuterium incorporation), at least 4500 times of the natural abundance of deuterium (67.5% deuterium incorporation), at least 5000 (75% deuterium), at least 5500 times of the natural abundance of deuterium (82.5% deuterium incorporation), at least 6000 times of the natural abundance of deuterium (90% deuterium incorporation), at least 6333.3 times of the natural abundance of deuterium (95% deuterium incorporation), at least 6466.7 times of the natural abundance of deuterium (97% deuterium incorporation), at least 6600 times of the natural abundance of deuterium (99% deuterium incorporation), or at least 6633.3 times of the natural abundance of deuterium (99.5% deuterium incorporation).

The percentage of deuterium incorporation can be obtained by quantitative analysis using a number of conventional methods, such as mass spectroscopy (peak area) or by quantifying the remaining residual ¹H-NMR signals of the specific deuteration site compared to signals from internal standards or other, non-deuterated ¹H signals in the compound.

When a chemical name or structure is silent as to whether a particular position in a compound normally occupied by hydrogen is isotopically enriched, it is intended that the particular position is occupied by hydrogen at its natural abundance. By way of example, the term “phenyl” or

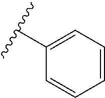 [SMILES: *c1ccccc1]

without any further designation as to isotopic enrichment indicates that all hydrogen atoms are present at natural abundance.

When ring A is a partially saturated cycle, the double bond in ring A is located in the depicted position:

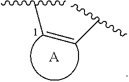

In case ring A is a 5-membered heteroaryl ring, then the double bond is within a delocated π-system and can exist in mesomeric forms. An example are the following thiophene mesomeric forms:

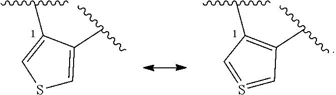 [SMILES: *c1cscc1* ; *C1=C(*)C=S=C1]

Furthermore, the compounds of the present invention are partly subject to tautomerism. For example, if a heteroaromatic group containing a nitrogen atom in the ring is substituted with a hydroxy group on the carbon atom adjacent to the nitrogen atom, the following tautomerism can appear:

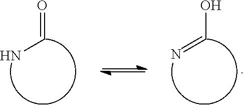

The term “1,4-orientation” (as mentioned for ring B) denotes the specific relative position of the two substituents on the same ring and means that on a ring the substituents have at least one possibility, where 4 atoms are between the two substituents in the ring attached to the ring system:

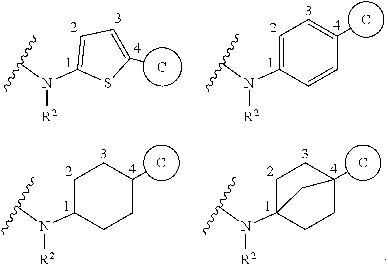

The term “compound,” when referring to any compound of this disclosure, including a compound represented by Formula (I) or a pharmaceutically acceptable salt and/or solvate thereof, refers to a collection of molecules having an identical chemical structure, except that there may be isotopic variation among the constituent hydrogen atoms of the molecules. The relative amount of isotopic variation in a compound of this invention will depend upon a number of factors including the isotopic purity of deuterated reagents used to make the compound and the efficiency of incorporation of deuterium in the various synthesis steps used to prepare the compound.

“D” and “d” both refer to deuterium. “H” refers to hydrogen.

“Substituted with deuterium” refers to the replacement of one or more hydrogen atoms with a corresponding number of deuterium atoms.

Any formula or structure given herein, is also intended to represent deuterated compounds comprising in addition further isotopically labelled atoms. Examples of additional isotopes that can be incorporated into compounds of the disclosure include further isotopes of hydrogen (i.e. tritium or ³H), as well as isotopes of carbon, nitrogen, oxygen, phosphorous, fluorine and chlorine, such as, but not limited to ¹¹C, ¹³C, ¹⁴C, ¹⁵N, ¹⁸F, ³¹P ³²P, ³⁵S, ³⁶Cl, and ¹²⁵I. The disclosure further comprises various isotopically labelled compounds into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or radioactive treatment of patients.

Halogen is selected from fluorine, chlorine, bromine and iodine, more preferably fluorine or chlorine and most preferably fluorine.

In the context of the present invention “C₁₋₄-alkyl” means a preferably saturated hydrocarbon chain having 1 to 4 carbon atoms which may be straight chained or branched. Examples thereof include methyl, ethyl, propyl, isopropyl, n-butyl, isobutyl and tert-butyl. Preferred is C₁₋₃-alkyl, such as methyl, ethyl, propyl and isopropyl, most preferred is methyl. The term “alkyl” by itself or as a part of another substituent, e.g. halo-C₁₋₄-alkyl, unless otherwise noted, is also meant to include those derivatives of alkyl defined in more detail below as “unsaturated alkyl”. An unsaturated alkyl group is one having one or more double bonds or triple bonds. Preferred unsaturated alkyl substituents are vinyl, 2-propenyl or prop-2-yn-1-yl.

In the context of the present invention the term “C₁₋₄-alkyl having one or more hydrogen atoms in alkyl optionally replaced by deuterium” encompasses, but is not limited to the following residues: -CD₃, —CH₂D, -CHD₂, CD₃CH₂(CH₂)ₙ—, CD₃CH₂(CHD)ₙ-, CD₃CH₂(CD₂)ₙ-, CH₂DCH₂(CH₂)ₙ-, CH₂DCH₂(CHD)ₙ-, CH₂DCH₂(CD₂)ₙ-, CHD₂CH₂(CH₂)ₙ-, CHD₂CH₂(CHD)ₙ-, CHD₂CH₂(CD₂)ₙ-, CD₃CHD(CH₂)ₙ-, CD₃CHD(CHD)ₙ-, CD₃CHD(CD₂)ₙ-, CH₂DCHD(CH₂)ₙ-, CH₂DCHD(CHD)ₙ-, CH₂DCHD(CD₂)ₙ-, CHD₂CHD(CH₂)ₙ-, CHD₂CHD(CHD)ₙ-, CHD₂CHD(CD₂)ₙ-, CH₃CHD(CH₂)ₙ-, CH₃CHD(CHD)ₙ-, CH₃CHD(CD₂)ₙ-, CD₃CD₂(CH₂)ₙ-, CD₃CD₂(CHD)ₙ-, CD₃CD₂(CD₂)ₙ-, CH₂DCD₂(CH₂)ₙ-, CH₂DCD₂(CHD)ₙ-, CH₂DCD₂(CD₂)ₙ-, CHD₂CD₂(CH₂)ₙ-, CHD₂CD₂(CHD)ₙ-, CHD₂CD₂(CD₂)ₙ-, CH₃CD₂(CH₂)ₙ-, CH₃CD₂(CHD)ₙ-, CH₃CD₂(CD₂)ₙ-, wherein n is an integer from 0 to 2, and CH₃CH₂(CHD)ₘ-, CH₃CH₂(CD₂)ₘ-, wherein m is an integer from 1 to 2, as well as -CD(CD₃)₂, —CH(CD₃)₂ and —C(CD₃)₃. Preferred C₁-2-alkyl containing deuterium are -CD₃ and -CD₃CD₂, most preferred is -CD₃.

A “C₀₋₆-alkylene” means that the respective group is divalent and connects the attached residue with the remaining part of the molecule. Moreover, in the context of the present invention, “C₀-alkylene” is meant to represent a bond, whereas C₁-alkylene means a methylene linker, C₂-alkylene means an ethylene linker or a methyl-substituted methylene linker and so on. In the context of the present invention, a C₀₋₆-alkylene preferably represents a bond, a methylene, an ethylene group or a propylene group. The term “alkylene”, unless otherwise noted, is also meant to include an unsaturated divalent chain, if appropriate (i.e. possible for “C₂₋₆-alkylene”). A representative example for an unsaturated C₄-alkylene is —CH₂—CH═CH—CH₂—.

The term “fluoro-C₁₋₄-alkyl” or “O-fluoro-C₁₋₄-alkyl”, respectively, means that one or more hydrogen atoms in the alkyl chain are replaced by one or more fluoro atoms. Preferred are CHF₂, CF₃, CH₂CF₃ and CF₂CF₃. A more preferred example thereof is the formation of a —CF₃ group.

Similar applies to “halo-C₁₋₄-alkyl” or “O-halo-C₁₋₄-alkyl”, which means that one or more hydrogen atoms in the alkyl chain are replaced by one or more halogen atoms, independently selected from fluoro, chloro, bromo and iodo.

In the context of the present invention the term “fluoro-C₁₋₄-alkyl having one or more hydrogen atoms in alkyl optionally replaced by deuterium” means, that if the fluoro-C₁₋₄-alkyl contains one or more hydrogen atom(s), one or more hydrogen(s) can be replaced by fluorine(s), yielding the same as described above for the term “C₁₋₄-alkyl having one or more hydrogen atoms in alkyl optionally replaced by deuterium”. It is understood, that fluoro-C₁₋₄-alkyl can also be completely fluorinated. Preferred are fluoro-C₁₋₂-alkyl containing deuterium such as CDF₂, CD₂CF₃ and CD₂CF₂D. Most preferred is CDF₂. A “3- to 10-membered cycloalkyl” group means a saturated or partially unsaturated mono-, bi-, spiro- or multicyclic ring system comprising 3 to 10 carbon atoms, wherein each of the atoms forming the ring system (i.e. skeletal atoms) is a carbon atom. Examples include cyclopropyl, cyclobutyl, cyclopentyl, cyclohexyl, cyclohexenyl, bicyclo[2.2.2]octyl, bicyclo[3.2.1]octanyl, spiro[3.3]heptyl, bicyclo[2.2.1]heptyl, adamantyl and pentacyclo[4.2.0.0^{2,5}.0^{3,8}.0^{4,7}]octyl. Consequently, a 3- to 6-membered cycloalkyl group means a saturated or partially unsaturated mono- bi-, or spirocyclic ring system comprising 3 to 6 carbon atoms whereas a 5- to 8-membered cycloalkyl group means a saturated or partially unsaturated mono-, bi-, or spirocyclic ring system comprising 5 to 8 carbon atoms.

The term “3- to 6-membered cycloalkyl” encompasses, but is not limited to cyclopropyl, cyclobutyl, cyclopentyl, cyclohexyl, bicyclo[1.1.1]pentyl, bicyclo[2.1.0]pentyl and spiro[2.3]hexanyl. More preferred is cyclopropyl or cyclobutyl.

The term “C₃₋₄-cycloalkyl” having one or more hydrogen atoms in alkyl optionally replaced by deuterium” encompasses, but is not limited to the following residues:

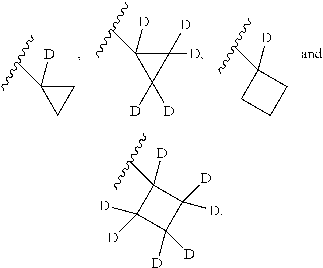

A cycloalkyl or heterocyclyl group can be connected straight or spirocyclic, e.g. when cyclohexane is substituted with the heterocycloalkyl group oxetane, the following structures are possible:

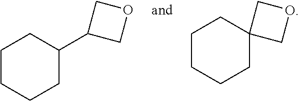 [SMILES: C1CCC(C2COC2)CC1 ; C1CCC2(CC1)COC2]

A “3- to 10-membered heterocycloalkyl containing 1 to 4 heteroatoms independently selected from N, O and S” group means a saturated or partially unsaturated 3 to 10 membered carbon mono-, bi-, spiro- or multicyclic ring wherein 1, 2, 3 or 4 carbon atoms are replaced by 1, 2, 3 or 4 heteroatoms, respectively, wherein the heteroatoms are independently selected from N, O or S. The sulfur heteroatom in the ring can also be oxidized to S═O or SO₂. The carbon atom in the ring can also be oxidized to C═O. Examples thereof include epoxidyl, oxetanyl, pyrrolidinyl, tetrahydrofuranyl, piperidinyl, piperazinyl tetrahydropyranyl, 1,4-dioxanyl, morpholinyl, 4-quinuclidinyl, 1,4-dihydropyridinyl and 6-azabicyclo[3.2.1]octanyl. The heterocycloalkyl group can be connected with the remaining part of the molecule via a carbon, nitrogen (e.g. in morpholine or piperidine) or sulfur atom. An example for a S-linked heterocycloalkyl is the cyclic sulfonimidamide

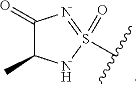 [SMILES: *S1(=O)=NC(=O)[C@H](C)N1]

The term “3- to 6-membered heterocycloalkyl” encompasses, but is not limited to epoxidyl, oxetanyl, pyrrolidinyl, tetrahydrofuranyl, piperidinyl, piperazinyl, 2-oxaspiro[3.3]heptyl, tetrahydropyranyl, 1,4-dioxanyl, morpholinyl and the like.

A “6- or 10-membered aryl” is phenyl or naphthyl.

A “5- to 10-membered heteroaryl containing 1 to 6 heteroatoms independently selected from N, O and S” means a 5- to 10-membered mono- or bicyclic heteroaromatic ring system (within the application also referred to as heteroaryl) containing up to 6 heteroatoms independently selected from N, O and S. Examples of monocyclic heteroaromatic rings include pyrrolyl, imidazolyl, furanyl, thiophenyl, pyridinyl, pyrimidinyl, pyrazinyl, pyrazolyl, oxazolyl, isoxazolyl, triazolyl, oxadiazolyl and thiadiazolyl. It further means a bicyclic ring system wherein the heteroatom(s) may be present in one or both rings including the bridgehead atoms. Examples thereof include quinolinyl, isoquinolinyl, quinoxalinyl, benzimidazolyl, benzisoxazolyl, benzofuranyl, benzoxazolyl, indolyl, indolizinyl 1,5-naphthyridinyl, 1,7-naphthyridinyl and pyrazolo[1,5-a]pyrimidinyl. The nitrogen or sulphur atom of the heteroaryl system may also be optionally oxidized to the corresponding N-oxide, S-oxide or S,S-dioxide. “5-membered heteroaryl” means a monocyclic aromatic ring system containing up to 3 heteroatoms independently selected from N, O and S. Examples of monocyclic heteroaromatic rings include pyrrolyl, imidazolyl, furanyl, thiophenyl and oxazolyl. The sulfur heteroatom in the ring can also be oxidized to S═O or SO₂.

A 5-membered heterocyclopentenyl group means a partially unsaturated 5-membered carbon monocyclic ring wherein 1 or 2 carbon atoms are replaced by 1 or 2 heteroatoms, respectively, wherein the heteroatoms are independently selected from N, O and S. Examples thereof include 2,3-dihydrofuranyl, 2,5-dihydrofuranyl, 2,5-dihydrothiophenyl or 2,5-dihydro-1H-pyrrole. The sulfur heteroatom in the ring can also be oxidized to S═O or SO₂.

The compounds of the invention may, depending on their structure, exist in tautomeric or stereoisomeric forms (enantiomers, diastereomers). The invention therefore also encompasses the tautomers, enantiomers or diastereomers and respective mixtures thereof. The stereoisomerically uniform constituents can be isolated in a known manner from such mixtures of enantiomers and/or diastereomers. The term “diastereomer” means stereoisomers that are not mirror images of one another and are non-superimposable on one another. The term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%. The compounds of the present invention can be in the form of a prodrug compound. “Prodrug” means a derivative that is converted into a compound according to the present invention by a reaction with an enzyme, gastric acid or the like under a physiological condition in the living body, e.g. by oxidation, reduction, hydrolysis or the like, each of which is carried out enzymatically. Other examples of the prodrug are compounds, wherein the carboxylic acid in a compound of the present invention is, for example, converted into an alkyl-, aryl-, arylalkylene-, amino-, choline-, acyloxyalkyl-, 1-((alkoxycarbonyl)oxy)-2-alkyl, or linolenoyl-ester. Exemplary structures for prodrugs of carboxylic acids are

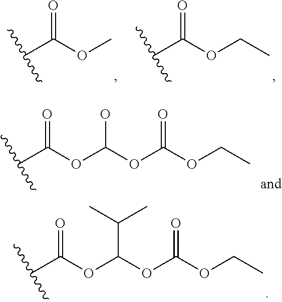

The term “pharmaceutically acceptable salts” refers to salts prepared from pharmaceutically acceptable non-toxic bases, including inorganic bases and organic bases. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The respective salts can be obtained by customary methods which are known to the person skilled in the art like, for example, by contacting these with an organic or inorganic base in a solvent or dispersant, or by cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts.

Further the compounds of the present disclosure may be present in the form of solvates, such as those which include as solvate water, or pharmaceutically acceptable solvates, such as alcohols, in particular ethanol. A stoichiometric or non-stoichiometric amount of solvent is bound by non-covalent intermolecular forces. When the solvent is water, the “solvate” is a “hydrate.” It is understood, that a “pharmaceutically acceptable salts” can in addition optionally contain a “solvate”.

The term “polymorph” as used herein refers to a crystalline form of a compound or a salt, hydrate, or solvate thereof, in a particular crystal packing arrangement. All polymorphs have the same elemental composition. The term “crystalline” as used herein, refers to a solid state form which consists of orderly arrangement of structural units. Different crystalline forms of the same compound, or a salt, hydrate, or solvate thereof, arise from different packing of the molecules in the solid state, which results in different crystal symmetries and/or unit cell parameter. Different crystalline forms usually have different X-ray diffraction patterns, infrared spectra, melting points, density, hardness, crystal shape, optical and electrical properties, stability, and solubility.

The term “effective amount” is meant to include the amount of a compound that, when administered, is sufficient to prevent development of, or alleviate to some extent, one or more of the symptoms of a disorder, disease, or condition being treated. The term “effective amount” also refers to the amount of a compound that is sufficient to elicit the biological or medical response of a cell, tissue, system, animal, or human, which is being sought by a researcher, veterinarian, medical doctor, or clinician.

As used herein, the term “subject” refers to any member of the animal kingdom including humans. In some embodiments, “subject” refers to humans, at any stage of development. In some embodiments, “subject” refers to a human patient. In some embodiments, “subject” refers to non-human animals. In some embodiments, the non-human animal is a mammal (e.g. a rodent, a mouse, a rat, a rabbit, a monkey, a dog, a cat, a sheep, cattle, a primate or a pig). In some embodiments, subjects include, but are not limited to, mammals, birds, reptiles, amphibians, fish or worms. In some embodiments, a subject may be a transgenic animal, genetically-engineered animal or a clone.

It has unexpectedly been found that deuterated compounds as detailed herein show higher microsomal stability and improved pharmacokinetic behavior in rat and mice. The following example section shows further details.

### Experimental Part

The compounds of the present invention can be prepared as outlined in WO2003/006425 and WO2004/056797 (and references cited therein) by using appropriate deuterated building blocks or via hydrogen-deuterium exchange (e.g. Synthesis 2019; 51:1319 or Angew. Chem. Int. Ed. 2018; 57:3022).

### Abbreviations

DBU 1,8-diazabicyclo[5.4.0]undec-7-ene
DMSO dimethylsulfoxide
dppf 1,1′-bis(diphenylphosphino)ferrocene
EA ethyl acetate
FCC flash chromatography on silica gel
PE petroleum ether
rt room temperature (20±4° C.)

### Experimental Section

### Preparative Example P1

### Step 1: 5-Bromo-1-fluoro-3-(methoxy-d3)-2-nitrobenzene (P1a)

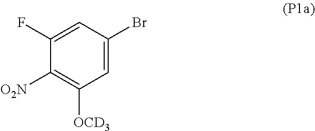

5-Bromo-1,3-difluoro-2-nitrobenzene can be treated with CD₃OD in KOH similar as described in WO2018/059314 to afford target compound P1a.

### Step 2: 4-Bromo-2-fluoro-6-(methoxy-d3)aniline (P1)

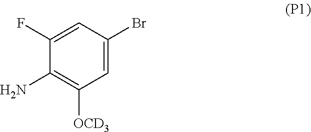

Compound P1a can be treated with hydrazine hydrate and Raney nickel as catalyst similar as described in WO2018/059314 to afford target compound P1.

### Preparative Example P2: 1-(3λ⁶-Propoxy-d9)-3-bromobenzene (P2)

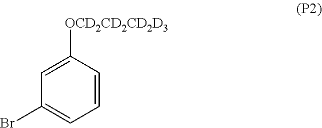

Compound P2 can be prepared by reacting 1-iodo-3λ⁶-propane-1,1,2,2,3,3,3,3,3-d9 with 3-bromophenol and potassium carbonate in DMF.

### Preparative Example P3: 2,6-Difluoro-4-(morpholino-d8)aniline (P3)

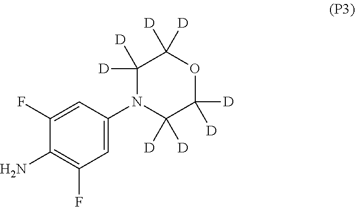 [SMILES: [2H]C1([2H])OC([2H])([2H])C([2H])([2H])N(c2cc(F)c(N)c(F)c2)C1([2H])[2H]]

By reacting tert-butyl (4-bromo-2,6-difluorophenyl)carbamate with morpholine-2,2,3,3,5,5,6,6-d8 using palladium diacetate, 2-dicyclohexylphosphino-2′,6′-dimethoxybiphenyl and potassium tert-butoxide in toluene for 14 h at 60° C. similar as described in WO2008/018426, target compound P3 can be prepared after deprotecting the Boc-group with 4N HCl in dioxane and aqueous workup under basic conditions.

### Preparative Example P4

### Step 1: 1-(1,3-dioxoisoindolin-2-yl) 4-methyl bicyclo[2.2.2]octane-1,4-dicarboxylate (P4a)

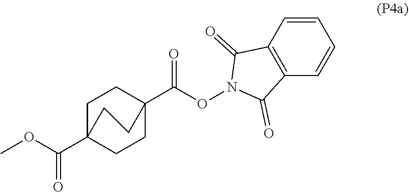 [SMILES: COC(=O)C12CCC(C(=O)ON3C(=O)c4ccccc4C3=O)(CC1)CC2]

4-(Methoxycarbonyl)bicyclo[2.2.2]octane-1-carboxylic acid was coupled with phthalimide using dicyclohexylcarbodiimide and 4-(dimethylamino)pyridine as catalyst in CH₂Cl₂ at rt.

### Step 2: Methyl 4-(3-methoxyphenyl)bicyclo[2.2.2]octane-1-carboxylate (P4b)

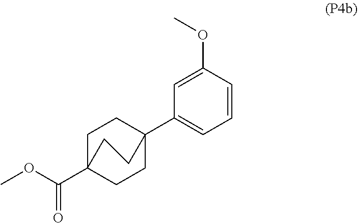 [SMILES: COC(=O)C12CCC(c3cccc(OC)c3)(CC1)CC2]

Compound P4a was coupled with bis(3-methoxyphenyl)zinc using 1,2-bis(diphenylphosphino)benzene and iron(III) acetylacetonate (Fe(acac)₃) as catalyst similar as outlined J. Am. Chem. Soc. 2016; 138:11132 to afford target compound P4b.

### Step 3: Methyl 4-(3-hydroxyphenyl)bicyclo[2.2.2]octane-1-carboxylate (P4c)

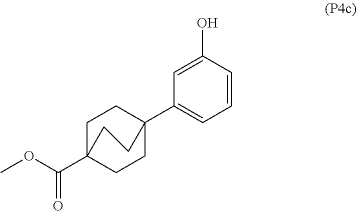 [SMILES: COC(=O)C12CCC(c3cccc(O)c3)(CC1)CC2]

Compound P4b was treated with BBr₃ in CH₂Cl₂ at −78° C. to rt to afford target compound P4c.

### Step 4: Methyl 4-(3-(methoxy-d3)phenyl)bicyclo[2.2.2]octane-1-carboxylate (P4d)

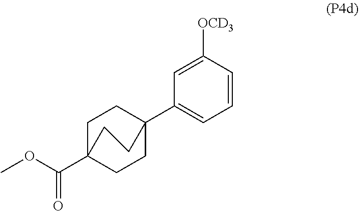

Compound P4c was alkylated with CD3I similar as described in Example 2, step 2 to give target compound P4d.

### Step 5: 4-(3-(Methoxy-d3)phenyl)bicyclo[2.2.2]octane-1-carboxylic acid (P4e)

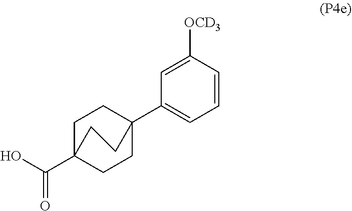

Compound P4d was saponified to afford target compound P4e.

### Step 6: Tert-Butyl (4-(3-(methoxy-d3)phenyl)bicyclo[2.2.2]octan-1-yl)carbamate (P4f)

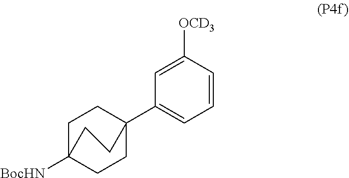

Compound P4e was treated with diphenylphosphoryl azide, Boc₂O and NEt₃ in tert-butanol under reflux for 16 h similar as outlined in WO2016/045587 to give target compound P4f.

### Step 7: 4-(3-(Methoxy-d3)phenyl)bicyclo[2.2.2]octan-1-amine (P4)

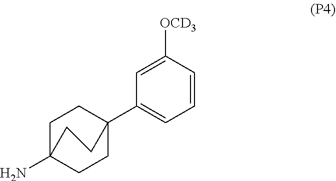

Compound P4e was treated with 4N HCl in dioxan to afford P4 after aqueous workup under basic conditions.

### Example 1

### Step 1: 2-Fluoro-4-(4,4,5,5-tetramethyl-1,3,2-dioxaborolan-2-yl)aniline (1a)

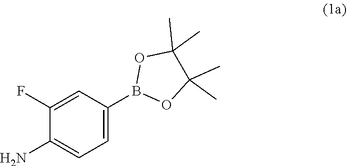 [SMILES: CC1(C)OB(c2ccc(N)c(F)c2)OC1(C)C]

To a solution of 4-bromo-2-fluoroaniline (4.00 g, 21.1 mmol) in 1,4-dioxane (30 mL) was added bis(pinacolato)diboron (5.38 g, 21.2 mmol), KOAc (6.23 g, 63.5 mmol) and Pd(dppf)Cl₂ (776 mg, 1.1 mmol). Then the mixture was heated at 90° C. for 1 h, cooled to rt, filtered, concentrated and purified by FCC (PE:EA=8:1) to give compound 1a as a white solid.

### Step 2: 3-Fluoro-3′-(methoxy-d3)-[1,1′-biphenyl]-4-amine (1b)

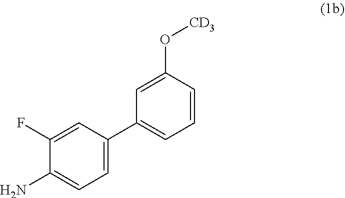 [SMILES: [2H]C([2H])([2H])Oc1cccc(-c2ccc(N)c(F)c2)c1]

To a solution of compound 1a (800 mg, 3.37 mmol) in 1,4-dioxane (10 mL) and H₂O (1 mL) was added 1-bromo-3-(methoxy-d3)benzene (638 mg, 3.36 mmol), Na₂CO₃ (1.07 g, 10.1 mmol) and Pd(dppf)Cl₂ (124 mg, 0.17 mmol) and then the mixture was heated at 90° C. for 2 h, cooled to rt, filtered, concentrated and purified by FCC (PE:EA=10:1) to give compound 1b as an oil.

### Step 3: 2-((3-Fluoro-3′-(methoxy-d3)-[1,1′-biphenyl]-4-yl)carbamoyl)cyclopent-1-ene-1-carboxylic Acid (1)

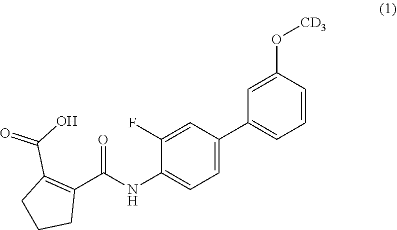 [SMILES: [2H]C([2H])([2H])Oc1cccc(-c2ccc(NC(=O)C3=C(C(=O)O)CCC3)c(F)c2)c1]

A solution of compound 1b (120 mg, 545 μmol) and 1-cyclopentene-1,2-dicarboxylic anhydride (74 mg, 540 μmol) in DCM (2.5 mL) was heated at 40° C. for 4 h. The mixture was filtered and the filter cake washed with MeCN (2×2 mL). The solid was dried in vacuum to afford compound 1 as a pale yellow solid. ¹H-NMR (400 MHz, DMSO-d6) δ 13.04 (br s, 1H), 10.58 (s, 1H), 8.07 (t, J=8.4 Hz, 1H), 7.63 (d, J=12.4 Hz, 1H), 7.53 (d, J=8.4 Hz, 1H), 7.37 (t, J=8.0 Hz, 1H), 7.27-7.23 (m, 2H), 6.94 (dd, J=8.0, 2.0 Hz, 1H), 2.80 (br s, 2H), 2.69 (br s, 2H), 1.93-1.85 (m, 2H). LCMS (ESI): m/z 359.0 (M+H)⁺.

### Example 1/1 to 1/13

The following Examples were prepared similar as described for Example 1 above using the appropriate building block as shown below.

| # | building block | structure | analytical data |
|---|---|---|---|
| 1/1 | 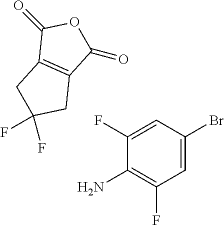 | 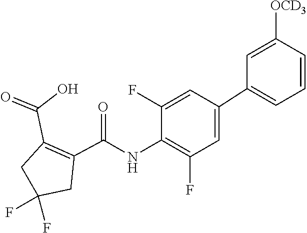 | ¹H-NMR (400 MHz, CD₃OD) δ 7.39-7.33 (m, 3H), 7.20 (d, J = 8.0 Hz, 1H), 7.16 (t, J = 2.0 Hz, 1H), 6.98-6.95 (m, 1H), 3.45- 3.27 (m, 4H). LCMS (ESI): m/z 413.0 (M + H)⁺ |
| 1/2 | 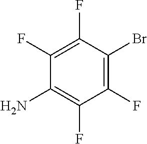 [SMILES: Nc1c(F)c(F)c(Br)c(F)c1F] | 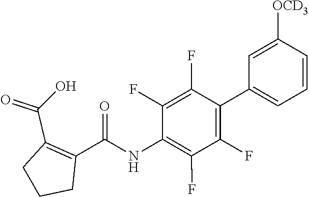 | ¹H-NMR (400 MHz, DMSO-d6) δ 12.88 (br s, 1H), 10.57 (s, 1H), 7.47 (t, J = 8.0 Hz, 1H), 7.12- 7.08 (m, 3H), 2.80 (t, J = 7.2 Hz, 2H), 2.68 (t, J = 7.2 Hz, 2H), 1.99-1.91 (m, 2H). LCMS (ESI): m/z 413.1 (M + H)⁺ |
| 1/3 | 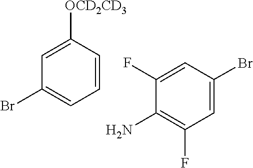 | 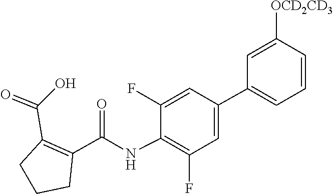 | ¹H-NMR (400 MHz, DMSO-d6) δ 12.95 (br s, 1H), 10.12 (s, 1H), 7.54 (t, J = 9.2 Hz, 2H), 7.38 (t, J = 8.0 Hz, 1H), 7.30-7.26 (m, 2H), 6.97 (dd, J = 2.8, 8.0 Hz, 1H), 2.80 (t, J = 7.2 Hz, 2H), 2.68 (t, J = 7.2 Hz, 2H), 1.96- 1.89 (m, 2H). LCMS (ESI): m/z 393.3 (M + H)⁺ |
| 1/4 | 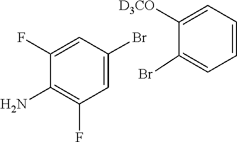 | 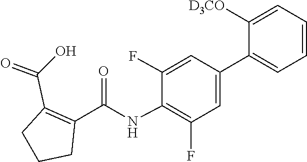 | ¹H-NMR (400 MHz, DMSO-d6) δ 10.09 (s, 1H), 7.42-7.39 (m, 2H), 7.28 (d, J = 8.8 Hz, 2H), 7.14 (d, J = 8.0 Hz, 1H), 7.07-7.02 (m, 1H), 2.82-2.78 (m, 2H), 2.70-2.65 (m, 2H), 1.97-1.88 (m, 2H). LCMS (ESI): m/z 377.3 (M + H)⁺ |
| 1/5 | 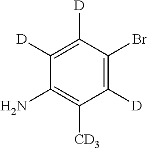 [SMILES: [2H]c1c([2H])c(Br)c([2H])c(C([2H])([2H])[2H])c1N] | 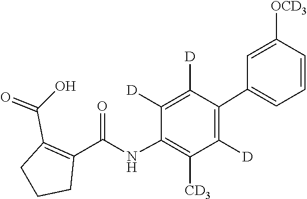 | ¹H-NMR (400 MHz, DMSO-d6) δ 13.14 (br s, 1H), 9.91 (s, 1H), 7.36 (t, J = 7.8 Hz, 1H), 7.38 (d, J = 8.0 Hz, 1H), 7.17 (t, J = 2.0 Hz, 1H), 6.93-6.89 (m, 1H), 2.82 (t, J = 7.4 Hz, 2H), 2.69 (t, J = 7.6 Hz, 2H), 1.96-1.88 (m, 2H). LCMS (ESI): m/z 361.1 (M + H)⁺ |
| 1/6 | 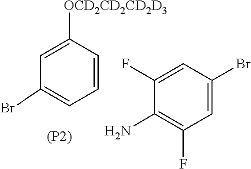 | 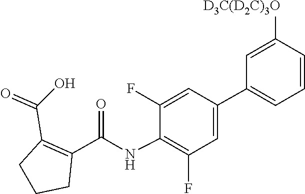 | ¹H-NMR (400 MHz, DMSO-d6) δ 12.96 (br s, 1H), 10.13 (s, 1H), 7.55 (d, J = 9.2 Hz, 2H), 7.38 (t, J = 7.8 Hz, 1H), 7.31-7.27 (m, 2H), 6.97 (dd, J = 1.8, 7.8 Hz, 1H), 2.82-2.78 (m, 2H), 2.70- 2.65 (m, 2H), 1.97-1.88 (m, 2H). LCMS (ESI): m/z 425.3 (M + H)⁺ |
| 1/7 | 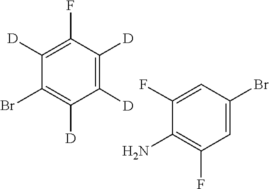 | 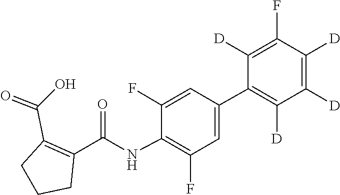 [SMILES: [2H]c1c([2H])c(F)c([2H])c(-c2cc(F)c(NC(=O)C3=C(C(=O)O)CCC3)c(F)c2)c1[2H]] | ¹H-NMR (400 MHz, CD₃OD) δ 7.38 (d, J = 9.5 Hz, 2H), 2.96- 2.91 (m, 2H), 2.87-2.82 (m, 2H), 2.04-1.96 (m, 2H). LCMS (ESI): m/z 366.1 (M + H)⁺ |
| 1/8 | 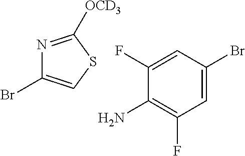 | 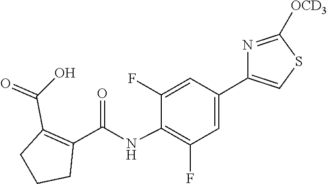 | ¹H-NMR (400 MHz, CD₃OD) δ 7.58-7.53 (m, 2H), 7.34 (s, 1H), 2.96-2.90 (m, 2H), 2.87-2.81 (m, 2H), 2.04-1.96 (m, 2H). LCMS (ESI): m/z 384.1 (M + H)⁺ |
| 1/9 | 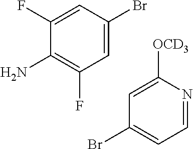 | 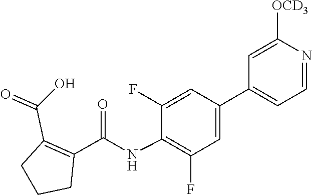 | ¹H-NMR (500 MHz, DMSO-d6) δ 12.90 (br s, 1H), 10.21 (s, 1H), 8.25 (d, J = 5.5 Hz, 1H), 7.69 (d, J = 8.5 Hz, 2H), 7.40 (d, J = 5.5 Hz, 1H), 7.23 (s, 1H), 2.81-2.77 (m, 2H), 2.69-2.65 (m, 2H), 1.96-1.89 (m, 2H). LCMS (ESI): m/z 378.1 (M + H)⁺ |
| 1/10 | 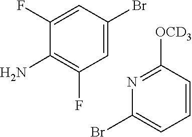 | 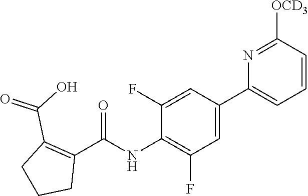 | ¹H-NMR (500 MHz, DMSO-d6) δ 12.98 (br s, 1H), 10.19 (s, 1H), 7.91 (d, J = 9.5 Hz, 2H), 7.83 (t, J = 7.8 Hz, 1H), 7.69 (d, J = 7.5 Hz, 1H), 6.85 (d, J = 8.5 Hz, 1H), 2.82-2.77 (m, 2H), 2.69-265 (m, 2H), 1.96-1.89 (m, 2H). LCMS (ESI): m/z 378.2 (M + H)⁺ |
| 1/11 | 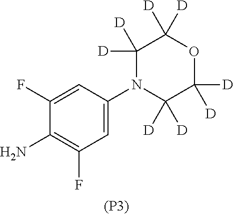 | 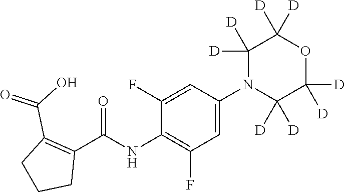 [SMILES: [2H]C1([2H])OC([2H])([2H])C([2H])([2H])N(c2cc(F)c(NC(=O)C3=C(C(=O)O)CCC3)c(F)c2)C1([2H])[2H]] | ¹H-NMR (400 MHz, CD₃OD) δ 6.64-6.59 (m, 2H), 2.96-2.89 (m, 2H), 2.87-2.79 (m, 2H), 2.03-1.94 (m, 2H). LCMS (ESI): m/z 361.2 (M + H)⁺ |
| 1/12 | 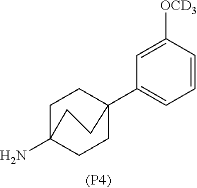 | 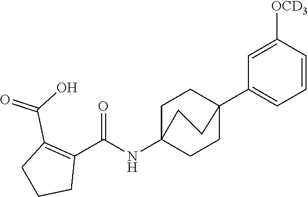 | ¹H-NMR (400 MHz, DMSO-d6) δ 8.83 (br s, 1H), 7.18 (t, J = 8.0 Hz, 1H), 6.89 (dd, J = 1.2, 8.8 Hz, 1H), 6.83 (t, J = 2.2 Hz, 1H), 6.73-6.71 (m, 1H), 2.65-2.57 (m, 4H), 1.97-1.93 (m, 6H), 1.85- 1.80 (m, 6H), 1.78-1.67 (m, 2H). LCMS (ESI): m/z 373.3 (M + H)⁺ |
| 1/13 | 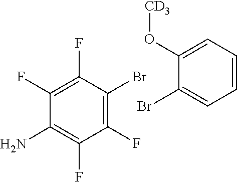 | 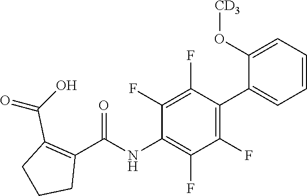 [SMILES: [2H]C([2H])([2H])Oc1ccccc1-c1c(F)c(F)c(NC(=O)C2=C(C(=O)O)CCC2)c(F)c1F] | ¹H-NMR (400 MHz, DMSO-d6) δ 12.84 (br s, 1H), 10.63 (br s, 1H), 7.55-7.50 (m, 1H), 7.39 (d, J = 6.4 Hz, 1H), 7.21 (d, J = 8.4 Hz, 1H), 7.10 (t, J = 7.2 Hz, 1H), 2.80 (t, J = 7.4 Hz, 2H), 2.68 (t, J = 7.4 Hz, 2H), 1.99-1.91 (m, 2H). LCMS (ESI): m/z 413.0 (M + H)⁺ |

### Example 2

### Step 1: 3-Bromophen-2,4,6-d3-ol (2a)

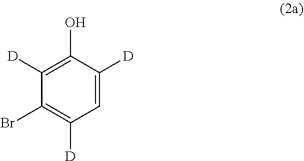 [SMILES: [2H]c1cc([2H])c(Br)c([2H])c1O]

A solution of 1-bromo-3-(methoxy-d3)benzene (800 mg, 4.21 mmol) in 20 mL of DCl (35% in D₂0) was heated in an autoclave at 105° C. for 2 days, cooled and diluted with Et₂O. The organic layer was separated, dried over Na₂SO₄, concentrated and purified by FCC (PE:EA=100:1 to 1:100) to afford compound 2a as an oil.

### Step 2: 1-Bromo-3-(methoxy-d3)benzene-2,4,6-d3 (2b)

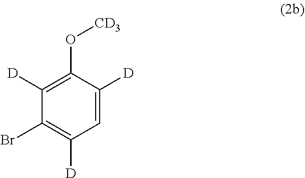 [SMILES: [2H]c1cc([2H])c(OC([2H])([2H])[2H])c([2H])c1Br]

To a solution of 2a (300 mg, 1.70 mmol) in MeCN (10 mL) was added iodomethane-d3 (0.13 mL, 2.1 mmol) and K₂CO₃ (472 mg, 3.41 mmol). The mixture was heated at 65° C. for 5 h, cooled to rt, filtered, concentrated and purified by FCC (PE:EA=20:1) to give compound 2b as an oil.

### Step 3: 3-Fluoro-3′-(methoxy-d3)-[1,1′-biphenyl]-2′,4′,6′-d3-4-amine (2c)

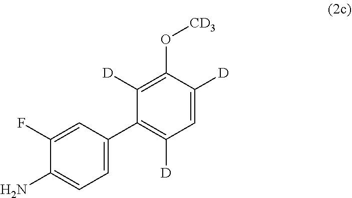 [SMILES: [2H]c1cc([2H])c(-c2ccc(N)c(F)c2)c([2H])c1OC([2H])([2H])[2H]]

To a solution of compound 1a (237 mg, 1.00 mmol) in 1,4-dioxane (5 mL) and H₂O (0.5 mL) was added compound 2b (192 mg, 994 μmol), Na₂CO₃ (0.32 g, 3.0 mmol) and Pd(dppf)Cl₂ (36 mg, 49 μmol) and then the mixture was heated at 90° C. for 2 h, cooled to rt, filtered, concentrated and purified by FCC (PE:EA=10:1) to give compound 2c as an oil.

### Step 4: 2-((3-Fluoro-3′-(methoxy-d3)-[1,1′-biphenyl]-4-yl-2′,4′,6′-d3)carbamoyl)cyclopent-1-ene-1-carboxylic Acid (2)

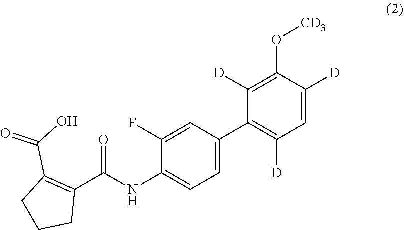 [SMILES: [2H]c1cc([2H])c(-c2ccc(NC(=O)C3=C(C(=O)O)CCC3)c(F)c2)c([2H])c1OC([2H])([2H])[2H]]

A solution of compound 2c (100 mg, 0.45 mmol) and 1-cyclopentene-1,2-dicarboxylic anhydride (62 mg, 0.45 mmol) in DCM (2.5 mL) was heated at 40° C. for 4 h. The mixture was filtered and the filter cake washed with MeCN (2×2 mL). The solid was dried in vacuum to afford compound 1b as a yellow solid. ¹H-NMR (500 MHz, DMSO-d6) δ 13.04 (br s, 1H), 10.57 (s, 1H), 8.07 (t, J=7.8 Hz, 1H), 7.63 (d, J=12.5 Hz, 1H), 7.53 (d, J=8.0 Hz, 1H), 7.37 (s, 1H), 2.80 (br s, 2H), 2.69 (br s, 2H), 1.93-1.85 (m, 2H). LCMS (ESI): m/z 362.0 (M+H)⁺.

### Example 2/1 to 2/2

The following Examples were prepared similar as described for Example 1 and 2 above using the appropriate building blocks as shown below.

| # | building block | structure | analytical data |
|---|---|---|---|
| 2/1 | 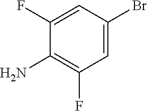 | 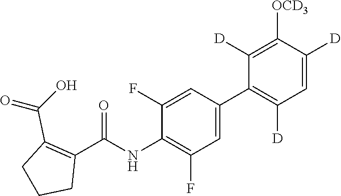 | ¹H-NMR (400 MHz, DMSO-d6) δ 12.94 (br s, 1H), 10.15 (s, 1H), 7.55 (d, J = 9.2 Hz, 2H), 7.39 (s, 1H), 2.83-2.77 (m, 2H), 2.70- 2.64 (m, 2H), 1.97-1.89 (m, 2H). LCMS (ESI): m/z 380.2 (M + H)⁺ |
| 2/2 | 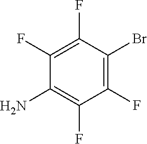 [SMILES: Nc1c(F)c(F)c(Br)c(F)c1F] | 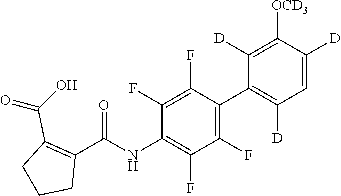 | ¹H-NMR (400 MHz, DMSO-d6) δ 12.86 (br s, 1H), 10.63 (br s, 1H), 7.46 (s, 1H), 2.82-2.76 (m, 2H), 2.70-2.64 (m, 2H), 1.98-1.91 (m, 2H). LCMS (ESI): m/z 380.2 (M + H)⁺ |

### Example 3

### Step 1: Ethyl 2-(3-bromophenoxy-2,4,6-d3)-2,2-difluoroacetate (3a)

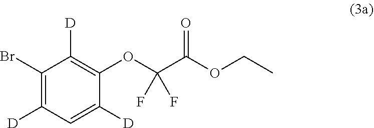 [SMILES: [2H]c1cc([2H])c(OC(F)(F)C(=O)OCC)c([2H])c1Br]

To a suspension of compound 2a (2.0 g, 11.4 mmol) and DBU (4.3 g, 28.2 mmol) in DMF (38 mL) was slowly added ethyl 2-bromo-2,2-difluoroacetate (5.8 g, 28.6 mmol) at rt. The mixture was stirred at rt for 16 h under a nitrogen atmosphere, poured into water (50 mL) and extracted with EA (3×100 mL). The combined organic layer was dried over Na₂SO₄, concentrated and purified by FCC (PE:EA=10:1) to give compound 3a as a colorless oil.

### Step 2: 2-(3-Bromophenoxy-2,4,6-d3)-2,2-difluoroacetic acid (3b)

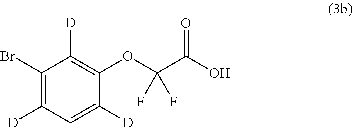 [SMILES: [2H]c1cc([2H])c(OC(F)(F)C(=O)O)c([2H])c1Br]

To a solution of compound 3a (2.8 g, 9.4 mmol) in MeOH (20 mL) and THF (5 mL) was added 3M aq. NaOH (5 mL). The mixture was stirred at rt for 30 min, acidified to pH 1, concentrated and purified by reversed-phase chromatography (C18) (0.1% NH₄HCO₃ in H₂O/MeCN=9:1 to 0:1 as gradient) to give compound 3b as an oil. LCMS (ESI): m/z 268.1 (M−H)⁻.

### Step 3: 1-Bromo-3-(trifluoromethoxy)benzene-2,4,6-d3 (3c)

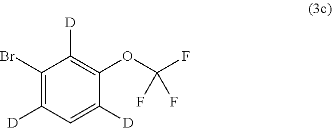 [SMILES: [2H]c1cc([2H])c(OC(F)(F)F)c([2H])c1Br]

To a solution of compound 3b (2.4 g, 8.9 mmol) in CDCl₃ (25 mL) was added XeF₂ (1.5 g, 8.8 mmol). The mixture was stirred at rt for 10 min, concentrated and purified by FCC (PE:EA=40:1) to give compound 3c as a colorless oil.

### Step 4: (3-(Trifluoromethoxy)phenyl-2,4,6-d3)boronic Acid (3d)

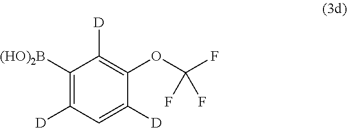 [SMILES: [2H]c1cc([2H])c(B(O)O)c([2H])c1OC(F)(F)F]

To a solution of compound 3c (1.3 g, 5.3 mmol) in dry THF (25 mL) was added n-BuLi (2.5 M, 2.1 mL, 5.3 mmol) at −78° C. The mixture was stirred for 30 min then a solution of triisopropyl borate (1.5 g, 8.0 mmol) in dry THF (5 mL) was added dropwise with stirring and cooling to keep the temperature at about −78° C. After addition, the mixture was to stirred for 30 min at this temperature, then allowed to warm to reach rt over a 1 h period. 2N HCl (3.1 mL) was added with stirring and the resulting mixture was concentrated and purified by reversed-phase chromatography (C18) (0.1% TFA in H₂O/MeCN=9:1 to 0:1 as gradient) to give compound 3d as a white solid. LCMS (ESI): m/z 208.2 (M−H)⁻.

### Step 5: 2,3,5,6-Tetrafluoro-3′-(trifluoromethoxy)-[1,1′-biphenyl]-2′,4′,6′-d3-4-amine (3e)

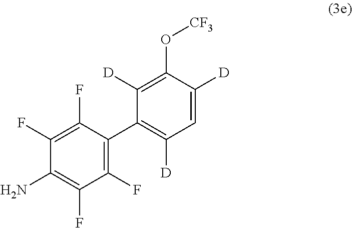 [SMILES: [2H]c1cc([2H])c(-c2c(F)c(F)c(N)c(F)c2F)c([2H])c1OC(F)(F)F]

To a solution of compound 3d (150 mg, 0.72 mmol) in 1,2-dimethoxyethane (3 mL) and H₂O (0.6 mL) was added 4-bromo-2,3,5,6-tetrafluoroaniline (174 mg, 0.71 mmol), Cs₂CO₃ (702 mg, 2.15 mmol) and Pd(PPh₃)₄ (46 mg, 40 μmol). The mixture was heated at 90° C. for 3 h, cooled and diluted with EA (20 mL). The organic layer was separated, dried over Na₂SO₄, concentrated and purified by FCC (PE:EA=8:1) to give compound 3e as a colorless oil. LCMS (ESI): m/z 329.2 (M+H)⁺.

### Step 6: 2-((2,3,5,6-Tetrafluoro-3′-(trifluoromethoxy)-[1,1′-biphenyl]-4-yl-2′,4′,6′-d3)carbamoyl)cyclopent-1-ene-1-carboxylic Acid (3)

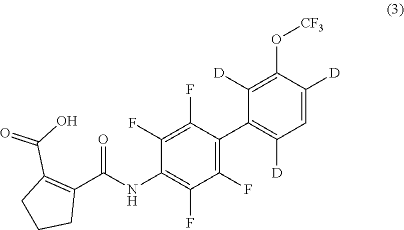 [SMILES: [2H]c1cc([2H])c(-c2c(F)c(F)c(NC(=O)C3=C(C(=O)O)CCC3)c(F)c2F)c([2H])c1OC(F)(F)F]

A solution of compound 3e (100 mg, 0.30 mmol) and 1-cyclopentene-1,2-dicarboxylic anhydride (50 mg, 0.36 mmol) in acetic acid (3 mL) was heated at 110° C. for 4 h, concentrated and purified by reversed-phase chromatography (C18) (0.1% NH₄HCO₃ in H₂O/MeCN=9:1 to 0:1 as gradient) to afford compound 3 as a white solid. ¹H-NMR (500 MHz, CD₃OD) δ 7.63 (s, 1H), 2.94-2.89 (m, 2H), 2.87-2.83 (m, 2H), 2.03-1.97. LCMS (ESI): m/z 467.1 (M+H)⁺, 489.2 (M+Na)⁺.

### Example 4

### Step 1: Methyl 3-(chlorocarbonyl)thiophene-2-carboxylate (4a)

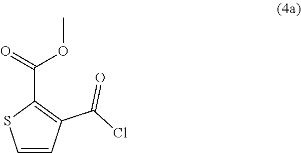

To a solution of 2-(methoxycarbonyl)thiophene-3-carboxylic acid (200 mg, 1.07 mmol) in dry DCM (8 mL) was added SOCl₂ (152 mg, 1.28 mmol). The reaction mixture was stirred at rt for 2 h and concentrated to give compound 4a as a yellow solid, which was used to next step without further purification.

### Step 2: 3-((2,3,5,6-Tetrafluoro-3′-(trifluoromethoxy)-[1,1′-biphenyl]-4-yl-2′,4′,6′-d3)carbamoyl)thiophene-2-carboxylic Acid (4)

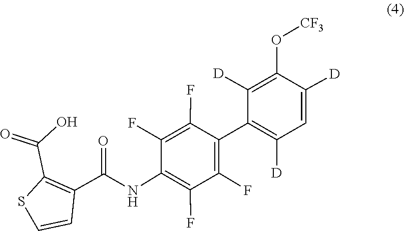 [SMILES: [2H]c1cc([2H])c(-c2c(F)c(F)c(NC(=O)c3ccsc3C(=O)O)c(F)c2F)c([2H])c1OC(F)(F)F]

To a solution of compound 3e (100 mg, 0.30 mmol) in dry THF (2 mL) was added NaH (60%, 30 mg, 0.75 mmol) at 0° C. The mixture was stirred for 1 h at this temperature, then a solution of compound 4a (150 mg, 0.73 mmol) in dry THF (1 mL) was added dropwise at a 0° C. After addition, the mixture was to stirred for 30 min at this temperature, then allowed to warm to rt and stirred for 1 h. Then water (1 mL) was added and stirring was continued for 1 h. Then the resulting mixture was concentrated and purified by reversed-phase chromatography (C18) (0.1% TFA in H₂O/MeCN=9:1 to 0:1 as gradient) to give compound 4 as a white solid. ¹H-NMR (400 MHz, DMSO-d6) δ 12.11 (br s, 1H), 11.35 (s, 1H), 7.94 (d, J=5.2 Hz, 1H), 7.71 (s, 1H), 7.44 (d, J=5.2 Hz, 1H). LCMS (ESI): m/z 483.1 (M+H)⁺, 505.1 (M+Na)³⁰.

### Example 4/1

The following Example was prepared similar as described for Example 4 above using the appropriate building block as shown below.

| # | building block | structure | analytical data |
|---|---|---|---|
| 4/1 | 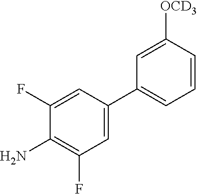 | 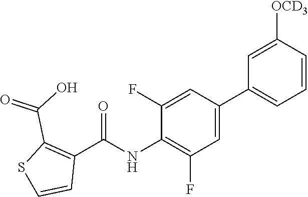 | ¹H-NMR (400 MHz, CD₃OD) δ 7.70 (d, J = 5.2 Hz, 1H), 7.47 (d, J = 5.2 Hz, 1H), 7.40-7.32 (m, 3H), 7.22-7.17 (m, 2H), 6.96 (dd, J = 2.0, 8.4 Hz, 1H). LCMS (ESI): m/z 393.1 (M + H)⁺ |

### Example 5

### Step 1: 1-Bromo-3-(difluoromethoxy-d)benzene (5a)

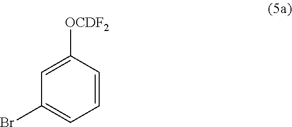

To a solution of 3-bromophenol (560 mg, 3.25 mmol) in dry THF (10 mL) was added NaH (1.3 g, 60% w/w, 33 mmol) at 0° C. and the mixture was stirred at 0° C. for 30 min, then D₂O (6.5 mL) was added dropwise at 0° C. for 10 min. After addition diethyl (bromodifluoromethyl)phosphonate (1.7 g, 6.5 mmol) the mixture was stirred at rt for 30 min. The mixture was extracted with EA (3×20 mL). The combined organic layers were washed with brine (100 mL), dried over Na₂SO₄, concentrated and purified by FCC (PE:EA=40:1) to give compound 5a as a colorless oil.

### Step 2: 3′-(Difluoromethoxy-d)-3-fluoro-[1,1′-biphenyl]-4-amine (5b)

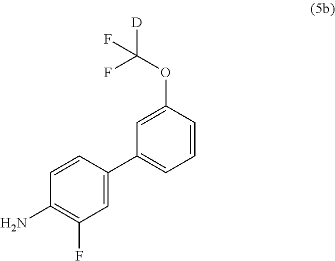 [SMILES: [2H]C(F)(F)Oc1cccc(-c2ccc(N)c(F)c2)c1]

To a solution of compound 5a (250 mg, 1.12 mmol) in 1,4-dioxane (6 mL) and H₂O (0.6 mL) was added compound 1a (265 mg, 1.12 mmol), Na₂CO₃ (356 mg, 3.36 mmol) and Pd(dppf)Cl₂ (41 mg, 0.06 mmol). The mixture was heated at 90° C. for 2 h and cooled. The organic layer was separated, concentrated and purified by FCC (PE:EA=10:1) to give compound 5b as a colorless oil.

### Step 3: 2-((3′-(Difluoromethoxy-d)-3-fluoro-[1,1′-biphenyl]-4-yl)carbamoyl)cyclopent-1-ene-1-carboxylic Acid (5)

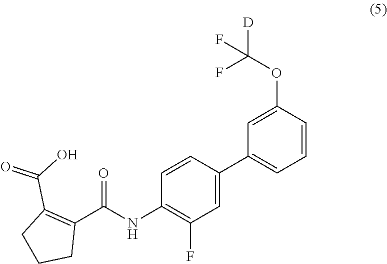 [SMILES: [2H]C(F)(F)Oc1cccc(-c2ccc(NC(=O)C3=C(C(=O)O)CCC3)c(F)c2)c1]

To a solution of compound 5b (70 mg, 0.28 mmol) in DCM (2.5 mL) was added 1-cyclopentene-1,2-dicarboxylic anhydride (39 mg, 0.28 mmol) and then the mixture was heated at 40° C. for 4 h. The mixture was cooled to rt, filtered and the filter cake washed with MeCN (2×2 mL). The solid was dried in vacuum to afford compound 5 as a light yellow solid. ¹H-NMR (500 MHz, DMSO-d6) δ 13.04 (br s, 1H), 10.70 (s, 1H), 8.12 (t, J=8.0 Hz, 1H), 7.68 (d, J=12.5 Hz, 1H), 7.60-7.50 (m, 4H), 7.18 (d, J=7.0, 1H), 2.80 (br s, 2H), 2.70 (br s, 2H), 1.92-1.86 (q, J=2.5 Hz, 2H). LCMS (ESI): m/z 393.3 (M+H)⁺.

### Example 6

### Step 1: 4-Bromo-2-fluorobenzen-6-d-amine (6a)

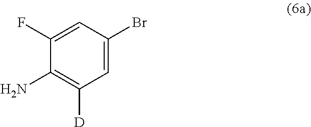

A solution of 4-bromo-2-fluoroaniline (2.0 g, 10.6 mmol) in 15 mL of DCl (35% in D₂0) in an autoclave was heated at 105° C. for 7 days. The solution was cooled to rt, adjusted with 6N NaOH to pH=8 and extracted with EA (3×20 mL). The combined organic layers were washed with brine (100 mL), dried over Na₂SO₄, concentrated and purified by FCC (PE:EA=10:1) to afford the compound 6a as an oil.

### Step 2: 2-Fluoro-4-(4,4,5,5-tetramethyl-1,3,2-dioxaborolan-2-yl)benzen-6-d-amine (6b)

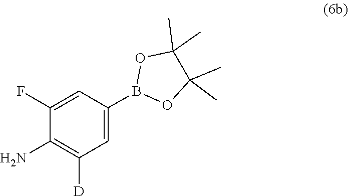 [SMILES: [2H]c1cc(B2OC(C)(C)C(C)(C)O2)cc(F)c1N]

To a solution of compound 6a (1.0 g, 5.3 mmol) in 1,4-dioxane (12 mL) was added bis(pinacolato)diboron (1.3 g, 5.3 mmol), KOAc (1.56 g, 15.9 mmol) and Pd(dppf)Cl₂ (190 mg, 0.26 mmol). The mixture was heated at 90° C. for 1 h, cooled to rt, filtered, concentrated and purified by FCC (PE:EA=10:1) to give compound 6b as a white solid.

### Step 3: 3-Fluoro-3′-(methoxy-d3)-[1,1′-biphenyl]-5-d-4-amine (6c)

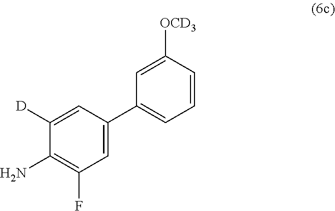

To a solution of compound 6b (380 mg, 1.60 mmol) in 1,4-dioxane (5 mL) and H₂O (0.5 mL) was added 1-bromo-3-(methoxy-d3)benzene (302 mg, 1.60 mmol), Na₂CO₃ (0.51 g, 4.8 mmol) and Pd(dppf)Cl₂ (58 mg, 0.08 mmol). The mixture was stirred at 90° C. for 2 h and then cooled to rt. The organic layer of was separated, concentrated and purified by FCC (PE:EA=10:1) to give compound 6c as an oil.

### Step 4: 2-((3-Fluoro-3′-(methoxy-d3)-[1,1′-biphenyl]-4-yl-5-d)carbamoyl)cyclopent-1-ene-1-carboxylic Acid (6)

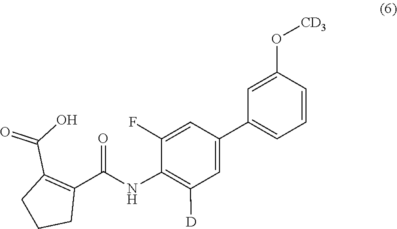 [SMILES: [2H]c1cc(-c2cccc(OC([2H])([2H])[2H])c2)cc(F)c1NC(=O)C1=C(C(=O)O)CCC1]

A solution of compound 6c (80 mg, 0.36 mmol) and 1-cyclopentene-1,2-dicarboxylic anhydride (50 mg, 0.36 mmol) in DCM (2.5 mL) was stirred at 40° C. for 4 h, cooled to rt and filtered. The filter cake washed with MeCN (2×2 mL). The solid was dried in vacuum to afford compound 6 as a yellow solid. ¹H-NMR (500 MHz, DMSO-d6) δ 13.04 (br s, 1H), 10.58 (s, 1H), 7.63 (dd, J=12.5, 2.0 Hz, 1H), 7.53 (d, J=1.5 Hz, 1H), 7.37 (t, J=8.0 Hz, 1H), 7.26 (d, J=8.5 Hz, 1H), 7.23-7.22 (m, 1H), 6.94 (dd, J=8.3, 2.3 Hz, 1H), 2.80 (t, J=7.3 Hz, 2H), 2.70 (t, J=7.3 Hz, 2H), 1.89 (p, J=7.6 Hz, 2H). LCMS (ESI): m/z 360.3 (M+H)⁺.

### Example 7: 2-((3-Fluoro-3′-hydroxy-[1,1′-biphenyl]-4-yl-2′,4′,5,6′-d4)carbamoyl)cyclopent-1-ene-1-carboxylic Acid (7)

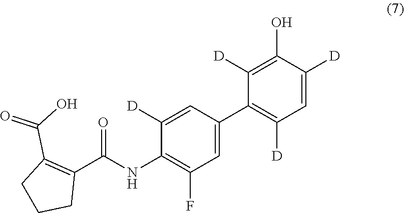 [SMILES: [2H]c1cc([2H])c(-c2cc([2H])c(NC(=O)C3=C(C(=O)O)CCC3)c(F)c2)c([2H])c1O]

By applying the route as outlined above by using the appropriate building blocks the target compound was obtained. ¹H-NMR (500 MHz, DMSO-d6) δ 13.03 (br s, 1H), 10.54 (s, 1H), 9.54 (s, 1H), 7.51 (dd, J=12.3, 1.8 Hz, 1H), 7.44 (d, J=1.5 Hz, 1H), 7.25 (s, 1H), 2.79 (t, J=7.0 Hz, 2H), 2.68 (t, J=7.0 Hz, 2H), 1.89 (p, J=7.0 Hz, 2H). LCMS (ESI): m/z 346.3 (M+H)⁺.

### Example 8: 2-((3-Fluoro-3′-hydroxy-[1,1′-biphenyl]-4-yl-2′,4′,6′-d3)carbamoyl)cyclopent-1-ene-1-carboxylic Acid (8)

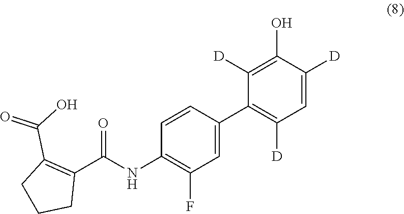 [SMILES: [2H]c1cc([2H])c(-c2ccc(NC(=O)C3=C(C(=O)O)CCC3)c(F)c2)c([2H])c1O]

By applying the route as outlined above by using the appropriate building blocks the target compound was obtained. ¹H-NMR (500 MHz, DMSO-d6) δ 10.68 (br s, 1H), 9.55 (s, 1H), 8.06 (dd, J=8.0, 9.0 Hz, 1H), 7.50 (d, J=12.0 Hz, 1H), 7.44 (d, J=8.0 Hz, 1H), 7.25 (s, 1H), 2.79-2.78 (m, 2H), 2.69-2.68 (m, 2H), 1.92-1.86 (m, 2H). LCMS (ESI): m/z 345.3 (M+H)⁺.

### Example 9

### Step 1: 2,6-Difluoro-4-(4,4,5,5-tetramethyl-1,3,2-dioxaborolan-2-yl)aniline (9a)

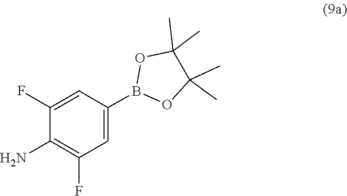 [SMILES: CC1(C)OB(c2cc(F)c(N)c(F)c2)OC1(C)C]

To a solution of 4-bromo-2,6-difluoroaniline (10 g, 48 mmol) in 1,4-dioxane (100 mL) was added bis(pinacolato)diboron (12.8 g, 50.4 mmol), CH₃COOK (14.1 g, 144 mmol) and Pd(dppf)Cl₂ (1.0 g, 2.40 mmol). The mixture was stirred at 90° C. under N₂ for 2 h, cooled to rt, concentrated and purified by FCC (PE:EA=10:1) to give compound 9a as a yellow solid.

### Step 2: 3,5-Difluoro-3′-(methoxy-d3)-[1,1′-biphenyl]-4-amine (9b)

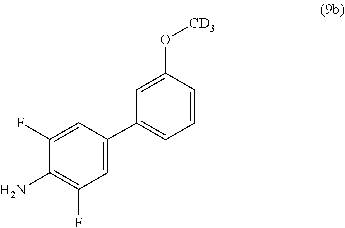 [SMILES: [2H]C([2H])([2H])Oc1cccc(-c2cc(F)c(N)c(F)c2)c1]

To a solution of compound 9a (4.5 g, 13.3 mmol) in 1,4-dioxane (50 mL) and H₂O (5 mL) was added 1-bromo-3-(methoxy-d3)benzene (3.34 g, 13.3 mmol), Na₂CO₃ (5.61 g, 39.4 mmol) and Pd(dppf)Cl₂ (400 mg, 0.67 mmol). The mixture was stirred at 90° C. under N₂ for 2 h, cooled to rt, concentrated and purified by FCC (PE:EA=10:1) to give compound 9b as a yellow solid. LCMS (ESI): m/z 239.1 (M+H)⁺.

### Step 3: 2-((3,5-Difluoro-3′-(methoxy-d3)-[1,1′-biphenyl]-4-yl)carbamoyl)cyclopent-1-ene-1-carboxylic Acid (9)

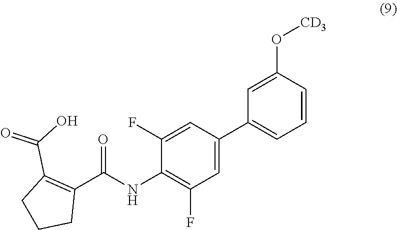 [SMILES: [2H]C([2H])([2H])Oc1cccc(-c2cc(F)c(NC(=O)C3=C(C(=O)O)CCC3)c(F)c2)c1]

To a solution of compound 9b (3.40 g, 14.3 mmol) in DCM (20 mL) were added 1-cyclopentene-1,2-dicarboxylic anhydride (1.90 g, 14.3 mmol) and then the mixture was stirred at rt for 2 h. The mixture was filtered and the filter cake washed with MeCN. The solid was dried in vacuum to afford compound 9 as a white solid. ¹H-NMR (500 MHz, DMSO-d6) δ 12.95 (br s, 1H), 10.13 (s, 1H), 7.55 (d, J=8.0 Hz, 2H), 7.39 (t, J=7.8 Hz, 1H), 7.32-7.28 (m, 2H), 6.99 (dd, J=1.8, 8.3 Hz, 1H), 2.81-2.79 (m, 2H), 2.69-2.66 (m, 2H), 1.97-1.89 (m, 2H). LCMS (ESI): m/z 377.3 (M+H)⁺.

### Example 10: 4-((3,5-Difluoro-3′-(methoxy-d3)-[1,1′-biphenyl]-4-yl)carbamoyl)-2,5-dihydro-thiophene-3-carboxylic Acid (10)

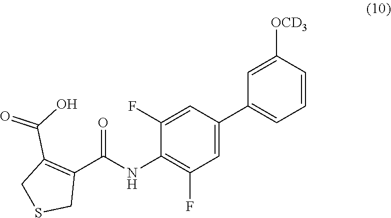

By reacting 4,6-dihydro-1H,3H-thieno[3,4-c]furan-1,3-dione (synthesis and coupling described in Bioorg. Med. Chem. Lett. 2005; 15:4854) similar as described above, the target molecule 10 was obtained. ¹H-NMR (400 MHz, DMSO-d6) δ 13.01 (br s, 1H), 10.20 (s, 1H), 7.54 (d, J=9.2 Hz, 2H), 7.39 (t, J=7.8 Hz, 1H), 7.32-2.28 (m, 2H), 6.99 (dd, J=2.4, 8.0 Hz, 1H), 4.15-4.11 (m, 2H), 4.03-4.00 (m, 2H). LCMS (ESI): m/z 395.2 (M+H)⁺.

### Example 11

### Step 1: 4,6-Dihydro-1H,3H-furo[3,4-c]furan-1,3-dione

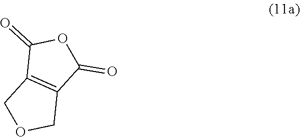

To a solution of 4,6-dihydro-1H,3H-thieno[3,4-c]furan-1,3-dione (400 mg, 2.23 mmol) (synthesis described in Bioorg. Med. Chem. Lett. 2005; 15:4854) in toluene (5 mL) was added AcCl (385 mg, 4.92 mmol) and then the mixture was stirred at 110° C. for 4 h, cooled to rt and concentrated under vacuum to afford compound 11a as a yellow solid, which was used for the next step without purification. LCMS (ESI): m/z=140.1 (M+H)⁺.

### Step 2: 4-((3,5-Difluoro-3′-(methoxy-d3)-[1,1′-biphenyl]-4-yl)carbamoyl)-2,5-dihydrofuran-3-carboxylic Acid (11)

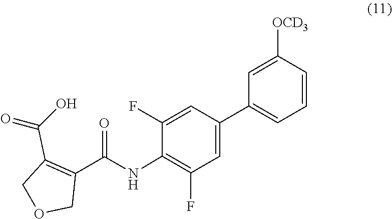

Compound 11a was reacted similar as described above to yield the target molecule 11 as a white solid. ¹H-NMR (500 MHz, DMSO-d6) δ 10.89 (br s, 1H), 7.58 (d, J=9.5 Hz, 2H), 7.39 (t, J=7.8 Hz, 1H), 7.33-2.28 (m, 2H), 6.99 (dd, J=2.3, 8.3 Hz, 1H), 4.97 (t, J=5.3 Hz, 2H), 4.89 (t, J=5.0 Hz, 2H), 3.43 (br s, 1H). LCMS (ESI): m/z 379.2 (M+H)⁺.

### Example 12 (Inverse Coupling Procedure)

### Step 1: 3-Fluoro-5-(3-(methoxy-d3)phenyl)pyridin-2-amine (12a)

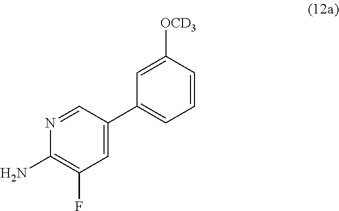

To a solution of 5-bromo-3-fluoropyridin-2-amine (400 mg, 2.09 mmol) in 1,4-dioxane (5 mL) and H₂O (0.5 mL) was added (3-(methoxy-d3)phenyl)boronic acid (389 mg, 2.51 mmol), Cs₂CO₃ (2.4 g, 6.27 mmol) and Pd(dppf)Cl₂ (40 mg, 0.11 mmol). The mixture was stirred at 90° C. under N₂ for 2 h and cooled to rt. The organic layer was separated, concentrated and purified by FCC (PE:EA=10:1) to give compound 12a as a yellow solid. LCMS (ESI): m/z 222.0 (M+H)⁺.

### Step 2: 2-((3-Fluoro-5-(3-(methoxy-d3)phenyl)pyridin-2-yl)carbamoyl)cyclopent-1-ene-1-carboxylic Acid (12)

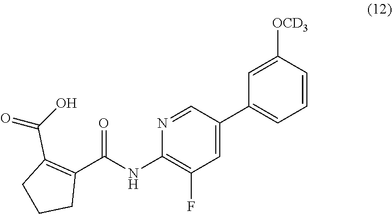

By reacting compound 12a as described in Example 6, step 4, the target molecule 12 was obtained. ¹H-NMR (500 MHz, MeOD) δ 8.50 (br s, (H), 7.99 (d, J=9.0 Hz, 8H), 7.41 (t, J=8.0 Hz, 1H), 7.25-7.21 (m, 2H), 6.99 (dd, J=8.5, 2.0 Hz, 1H), 2.97-2.93 (i, 2H), 2.86-2.83 (m, 2H), 2.03-1.97 (m, 2H). LCMS (ESI): m/z 360.1 (M+H)⁺.

### Example 12/1 to 12/6

The following Examples were prepared similar as described for Example 12 above using the appropriate building blocks as shown below.

| # | building block | structure | analytical data |
|---|---|---|---|
| 12/1 | 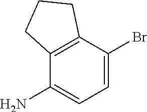 [SMILES: Nc1ccc(Br)c2c1CCC2] | 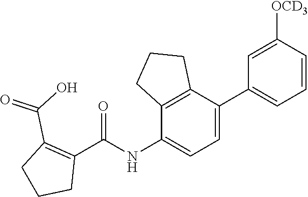 | ¹H-NMR (500 MHz, DMSO-d6) δ 13.11 (br s, 1H), 10.11 (s, 1H), 7.59 (d, J = 8.0 Hz, 1H), 7.34 (t, J = 7.8 Hz, 1H), 7.18 (d, J = 8.0 Hz, 1H), 7.01 (d, J = 8.0 Hz, 1H), 6.96 (t, J = 1.8 Hz, 1H), 6.90 (dd, J = 8.0, 2.0 Hz, 1H), 2.96-2.87 (m, 4H), 2.83-2.80 (m, 2H), 2.72-2.68 (m, 2H), 2.00-1.86 (m, 4H). LCMS (ESI): m/z 381.3 (M + H)⁺ |
| 12/2 | 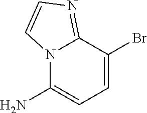 [SMILES: Nc1ccc(Br)c2nccn12] | 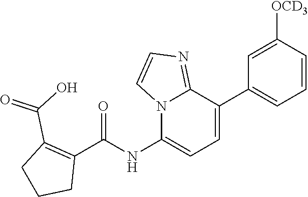 | ¹H-NMR (400 MHz, CD₃OD) δ 8.65 (d, J = 1.6 Hz, 1H), 8.10 (d, J = 2.0 Hz, 1H), 8.04 (d, J = 9.2 Hz, 1H), 7.98 (d, J = 9.6 Hz, 1H), 7.45 (t, J = 7.8 Hz, 1H), 7.08-6.99 (m, 3H), 2.76 (t, J = 7.6 Hz, 2H), 2.68 (t, J = 7.4 Hz, 2H), 2.03-1.95 (m, 2H). LCMS (ESI): m/z 381.2 (M + H)⁺ |
| 12/3 | 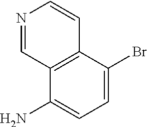 [SMILES: Nc1ccc(Br)c2ccncc12] | 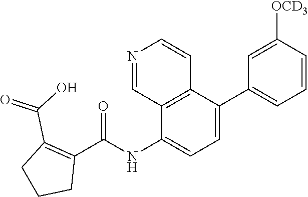 | ¹H-NMR (500 MHz, DMSO-d6) δ 10.85 (s, 1H), 9.69 (s, 1H), 8.56 (d, J = 4.0 Hz, 1H), 7.97-7.88 (m, 3H), 7.49 (t, J = 8.0 Hz, 1H), 7.10-7.06 (m, 3H), 2.90 (br s, 2H), 2.73 (br s, 2H), 2.02-1.96 (m, 2H). LCMS (ESI): m/z 392.2 (M + H)⁺ |
| 12/4 | 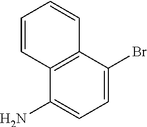 [SMILES: Nc1ccc(Br)c2ccccc12] | 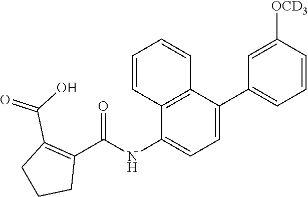 | ¹H-NMR (500 MHz, DMSO-d6) δ 13.11 (br s, 1H), 10.49 (s, 1H), 8.23 (d, J = 7.5 Hz, 1H), 7.85 (d, J = 8.5 Hz, 1H), 7.72 (d, J = 7.5 Hz, 1H), 7.57-7.43 (m, 4H), 7.05-7.00 (m, 3H), 2.92-2.89 (m, 2H), 2.75-2.71 (m, 2H), 2.00-1.93 (m, 2H). LCMS (ESI): m/z 391.2 (M + H)⁺ |
| 12/5 | 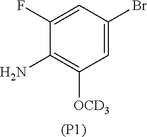 | 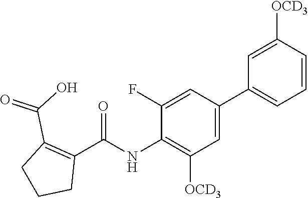 | ¹H-NMR (400 MHz, DMSO-d6) δ 13.48 (br s, 1H), 9.92 (s, 1H), 7.39 (d, J = 8.0 Hz, 1H), 7.31 (d, J = 8.0 Hz, 1H), 7.27 (d, J = 6.4 Hz, 1H), 7.21-7.16 (m, 2H), 6.98 (dd, J = 2.2, 8.2 Hz, 1H), 2.84-2.80 (m, 2H), 2.73-2.68 (m, 2H), 1.93-1.85 (m, 2H). LCMS (ESI): m/z 392.1 (M + H)⁺ |
| 12/6 | 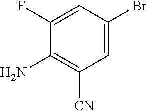 [SMILES: N#Cc1cc(Br)cc(F)c1N] | 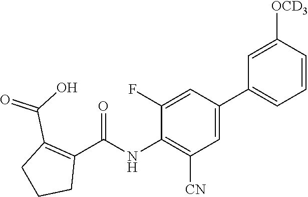 | LCMS (ESI): m/z 384.1 (M + H)⁺ |

### Example 13

### Step 1: Di-Tert-Butyl (3,5-difluoro-3′-(methoxy-d3)-[1,1′-biphenyl]-4-yl)iminodicarbonate (13a)

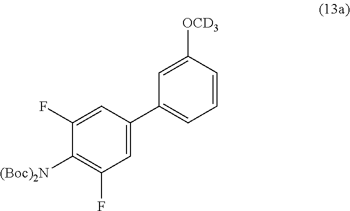

By treating compound 9b with di-tert-butyl dicarbonate in DMF and with DMAP as catalyst similar as described in WO2008/018426, the target compound 13a was prepared.

### Step 2: tert-Butyl (3,5-difluoro-3′-(methoxy-d3)-[1,1′-biphenyl]-4-yl)carbamate (13b)

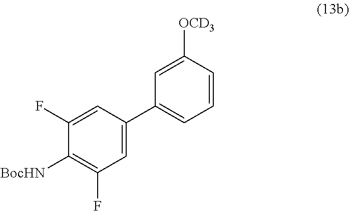

By treating compound 13a with trifluoroacetic acid in CH₂Cl₂ at 0° C. similar as described in Chem. Commun. 2018; 54:4589, the target compound 13b was prepared.

### Step 3: Tert-Butyl (3,5-difluoro-3′-(methoxy-d3)-[1,1′-biphenyl]-4-yl)(methyl)carbamate (13c)

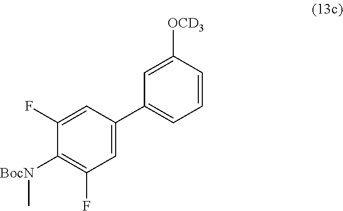

By treating compound 13b with lithium bis(trimethylsilyl)amide and Mel similar as described in J. μm. Chem. Soc. 2002; 124:8206, the target compound 13c was prepared.

### Step 4: 3,5-Difluoro-3′-(methoxy-d3)-N-methyl-[1,1′-biphenyl]-4-amine (13d)

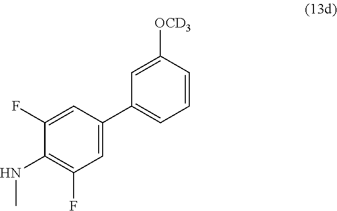

By deprotecting compound 13c with 4N HCl in dioxane the target compound 13d was prepared after workup under basic conditions.

### Step 5: 2-((3,5-Difluoro-3′-(methoxy-d3)-[1,1′-biphenyl]-4-yl)(methyl)carbamoyl)cyclopent-1-ene-1-carboxylic Acid (13)

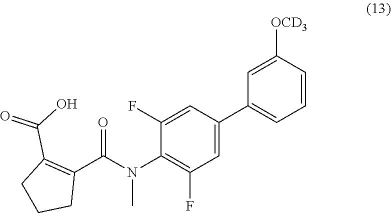

By coupling compound 13d with 1-cyclopentene-1,2-dicarboxylic anhydride similar as described above, the target compound 13 was prepared. LCMS (ESI): m/z 391.1 (M+H)⁺.

### Example 14

### Step 1: Methyl (3,5-difluoro-3′-(methoxy-d3)-[1,1′-biphenyl]-4-yl)glycinate (14a)

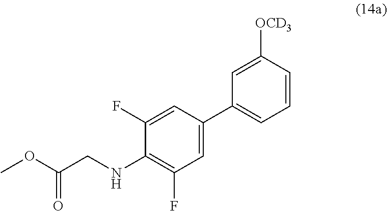

By treating compound 9b with methyl 2-bromoacetate, the target compound 14a can be prepared.

### Step 2: 2-((3,5-Difluoro-3′-(methoxy-d3)-[1,1′-biphenyl]-4-yl)(2-methoxy-2-oxoethyl)carbamoyl)cyclopent-1-ene-1-carboxylic Acid (14)

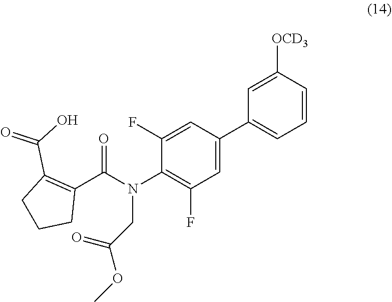

By coupling compound 14a with 1-cyclopentene-1,2-dicarboxylic anhydride similar as described above, the target compound 14 can be prepared.

### Example 15: 2-((3,5-Difluoro-3′-(methoxy-d3)-[1,1′-biphenyl]-4-yl)(2-hydroxyethyl)carbamoyl) cyclopent-1-ene-1-carboxylic acid (15)

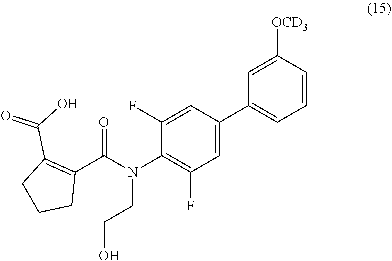

By reducing compound 14a with e.g. lithium borohydride, the target compound 15 can be prepared.

### Example 99: 2-((3-Fluoro-3′-(methoxy-d3)-[1,1′-biphenyl]-4-yl)carbamoyl)cyclopent-1-ene-1-carboxylic-3,3,4,4,5,5-d6 Acid

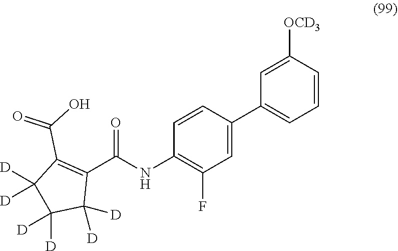

By bis-esterification of hexanedioic-d8 acid (CAS number: 52089-65-3) with MeOH, dimethyl hexanedioate-d8 can be obtained. This bis-ester can be cyclized as described in WO2009/140279 to afford methyl 2-hydroxycyclopent-1-ene-1-carboxylate-3,3,4,4,5,5-d6, of which the corresponding triflate can be prepared. By Palladium-catalysed reaction with sodium formate as described in Heterocycles 2009; 77:179 mono-acid cyclopent-1-ene-1,2-dicarboxylic-d6 acid can be obtained, which can be coupled with compound 1b to afford the target molecule.

### Example 100/1 to 100/13

The following Examples can be prepared similar as described for the Examples above using the appropriate building blocks as shown below.

| # | building block | structure |
|---|---|---|
| 100/1 | 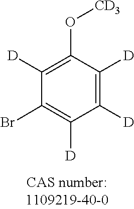 [SMILES: [2H]c1c([2H])c(Br)c([2H])c(OC([2H])([2H])[2H])c1[2H]] | 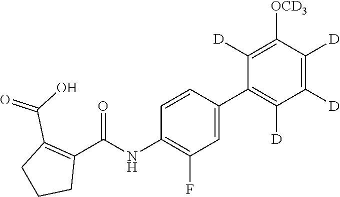 |
| 100/2 | 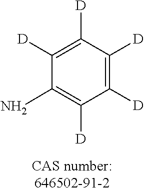 [SMILES: [2H]c1c([2H])c([2H])c(N)c([2H])c1[2H]] | 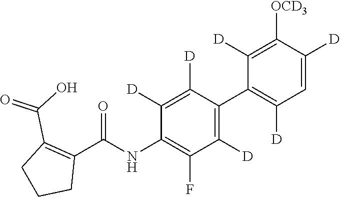 |
| 100/3 | 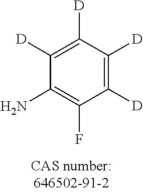 [SMILES: [2H]c1c([2H])c([2H])c(F)c(N)c1[2H]] | 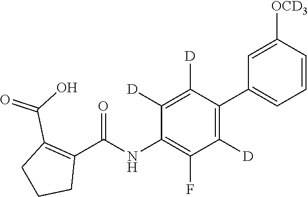 |
| 100/4 | 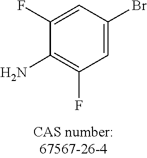 | 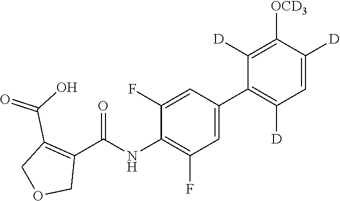 |
| 100/5 | 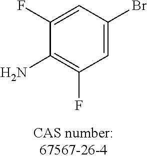 | 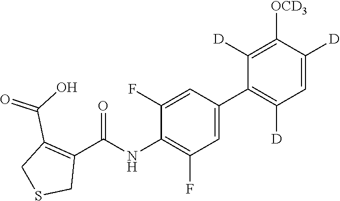 |
| 100/6 | 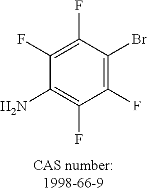 [SMILES: Nc1c(F)c(F)c(Br)c(F)c1F] | 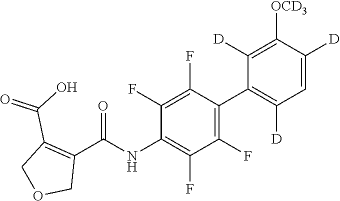 |
| 100/7 | 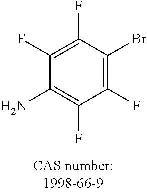 [SMILES: Nc1c(F)c(F)c(Br)c(F)c1F] | 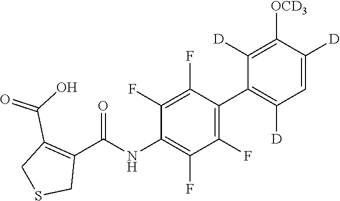 |
| 100/8 | 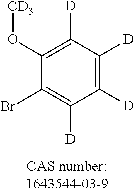 [SMILES: [2H]c1c([2H])c([2H])c(OC([2H])([2H])[2H])c(Br)c1[2H]] | 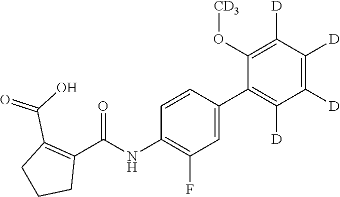 [SMILES: [2H]c1c([2H])c([2H])c(-c2ccc(NC(=O)C3=C(C(=O)O)CCC3)c(F)c2)c(OC([2H])([2H])[2H])c1[2H]] |
| 100/9 | 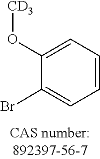 [SMILES: [2H]C([2H])([2H])Oc1ccccc1Br] | 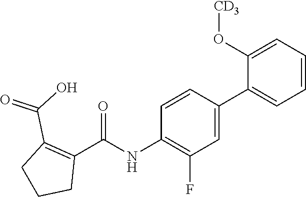 [SMILES: [2H]C([2H])([2H])Oc1ccccc1-c1ccc(NC(=O)C2=C(C(=O)O)CCC2)c(F)c1] |
| 100/10 | 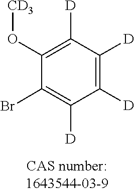 [SMILES: [2H]c1c([2H])c([2H])c(OC([2H])([2H])[2H])c(Br)c1[2H]] | 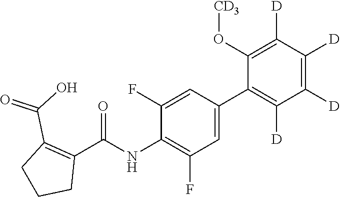 [SMILES: [2H]c1c([2H])c([2H])c(-c2cc(F)c(NC(=O)C3=C(C(=O)O)CCC3)c(F)c2)c(OC([2H])([2H])[2H])c1[2H]] |
| 100/11 | 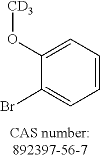 [SMILES: [2H]C([2H])([2H])Oc1ccccc1Br] | 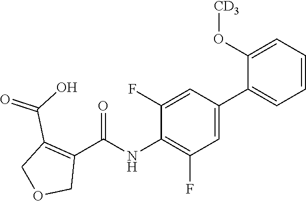 [SMILES: [2H]C([2H])([2H])Oc1ccccc1-c1cc(F)c(NC(=O)C2=C(C(=O)O)COC2)c(F)c1] |
| 100/12 | 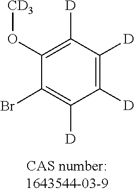 [SMILES: [2H]c1c([2H])c([2H])c(OC([2H])([2H])[2H])c(Br)c1[2H]] | 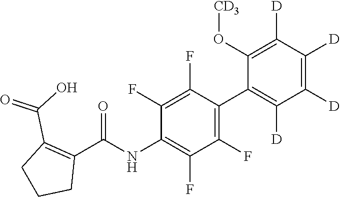 [SMILES: [2H]c1c([2H])c([2H])c(-c2c(F)c(F)c(NC(=O)C3=C(C(=O)O)CCC3)c(F)c2F)c(OC([2H])([2H])[2H])c1[2H]] |
| 100/13 | 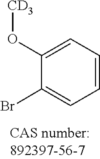 [SMILES: [2H]C([2H])([2H])Oc1ccccc1Br] | 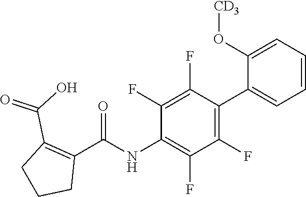 [SMILES: [2H]C([2H])([2H])Oc1ccccc1-c1c(F)c(F)c(NC(=O)C2=C(C(=O)O)CCC2)c(F)c1F] |

### Example 101/1 to 101/16

The following Examples can be prepared similar as described for the Examples above using the appropriate building blocks as shown below.

| # | building block | structure |
|---|---|---|
| 101/1 | 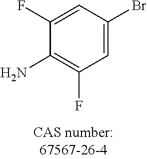 | 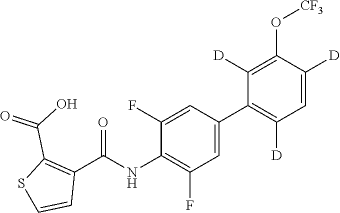 [SMILES: [2H]c1cc([2H])c(-c2cc(F)c(NC(=O)c3ccsc3C(=O)O)c(F)c2)c([2H])c1OC(F)(F)F] |
| 101/2 | alkylation of compound 2a | 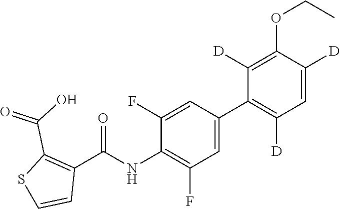 [SMILES: [2H]c1cc([2H])c(-c2cc(F)c(NC(=O)c3ccsc3C(=O)O)c(F)c2)c([2H])c1OCC] |
| 101/3 | alkylation of compound 2a | 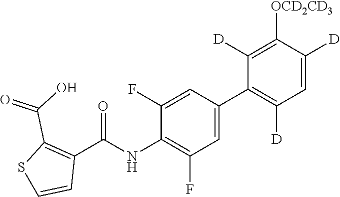 |
| 101/4 | 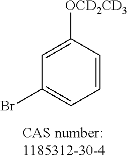 | 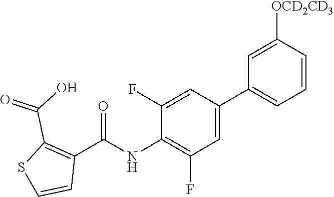 |
| 101/5 | alkylation of compound 2a | 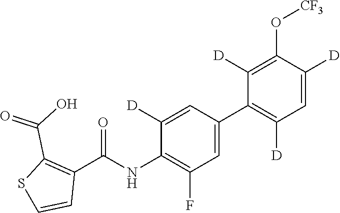 [SMILES: [2H]c1cc(-c2c([2H])cc([2H])c(OC(F)(F)F)c2[2H])cc(F)c1NC(=O)c1ccsc1C(=O)O] |
| 101/6 | compound 6b | 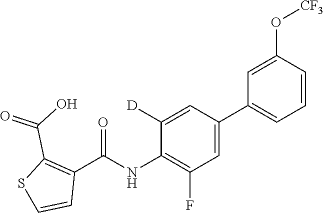 [SMILES: [2H]c1cc(-c2cccc(OC(F)(F)F)c2)cc(F)c1NC(=O)c1ccsc1C(=O)O] |
| 101/7 | 1-bromo-3- (methoxy- d3)benzene | 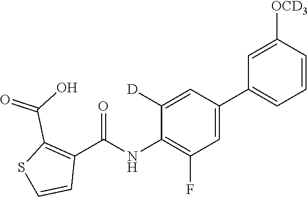 |
| 101/8 | 1-bromo-3- (methoxy- d3)benzene | 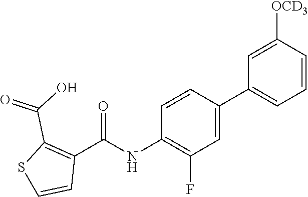 |
| 101/9 | alkylation of compound 2a | 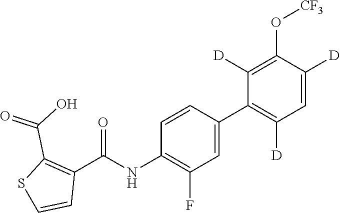 [SMILES: [2H]c1cc([2H])c(-c2ccc(NC(=O)c3ccsc3C(=O)O)c(F)c2)c([2H])c1OC(F)(F)F] |
| 101/10 | 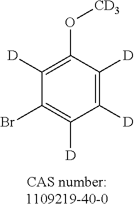 [SMILES: [2H]c1c([2H])c(Br)c([2H])c(OC([2H])([2H])[2H])c1[2H]] | 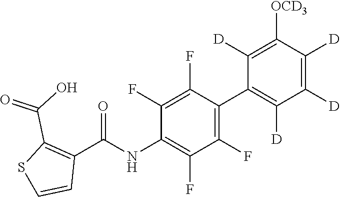 |
| 101/11 | compound 5a | 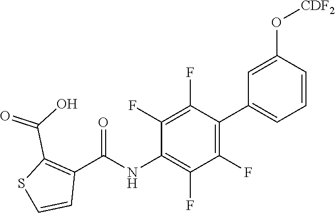 |
| 101/12 | alkylation of compound 2a | 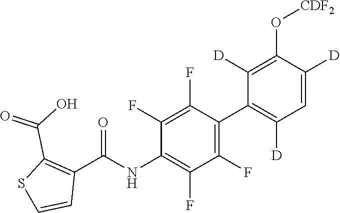 |
| 101/13 | 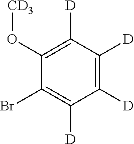 [SMILES: [2H]c1c([2H])c([2H])c(OC([2H])([2H])[2H])c(Br)c1[2H]] | 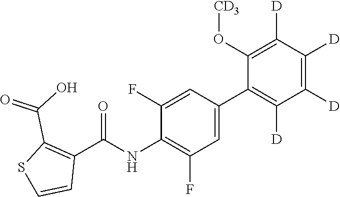 [SMILES: [2H]c1c([2H])c([2H])c(-c2cc(F)c(NC(=O)c3ccsc3C(=O)O)c(F)c2)c(OC([2H])([2H])[2H])c1[2H]] |
| 101/14 | 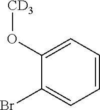 [SMILES: [2H]C([2H])([2H])Oc1ccccc1Br] | 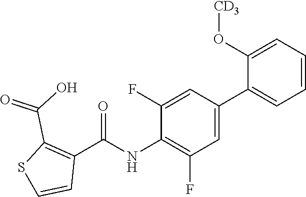 [SMILES: [2H]C([2H])([2H])Oc1ccccc1-c1cc(F)c(NC(=O)c2ccsc2C(=O)O)c(F)c1] |
| 101/15 | 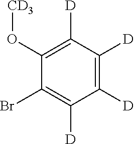 [SMILES: [2H]c1c([2H])c([2H])c(OC([2H])([2H])[2H])c(Br)c1[2H]] | 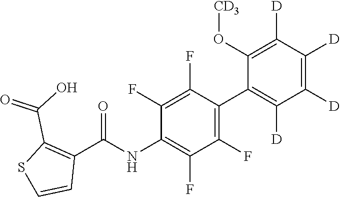 [SMILES: [2H]c1c([2H])c([2H])c(-c2c(F)c(F)c(NC(=O)c3ccsc3C(=O)O)c(F)c2F)c(OC([2H])([2H])[2H])c1[2H]] |
| 101/16 | 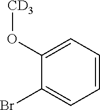 [SMILES: [2H]C([2H])([2H])Oc1ccccc1Br] | 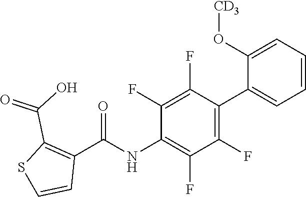 [SMILES: [2H]C([2H])([2H])Oc1ccccc1-c1c(F)c(F)c(NC(=O)c2ccsc2C(=O)O)c(F)c1F] |

### Example 200: Human DHODH Inhibition Assay

The in vitro inhibition of hDHODH was measured using an N-terminally truncated recombinant hDHODH enzyme as described in J. Med. Chem. 2006; 49:1239. Briefly, the hDHODH concentration was adjusted in a way that an average slope of approximately 0.2 AU/min served as the positive control (e.g. without inhibitor). The standard assay mixture contained 60 μM 2,6-dichloroindophenol, 50 μM decylubiquinone and 100 μM dihydroorotate. The hDHODH enzyme with or without at least six different concentrations of the compounds was added and measurements were performed in 50 mM TrisHCl, 150 mM KCl and 0.1% Triton X-100 at pH 8.0 and at 30° C. The reaction was started by adding dihydroorotate and measuring the absorption at 600 nm for 2 min. For the determination of the IC₅₀ values, each data point was recorded in triplicate. For the determination of the inhibitory constant Kᵢ, the K_{M} values for DHO and decylubichinon were determined. Afterwards, the compounds were diluted in a dilution series depending on their IC₅₀ values in DMSO. The dilution was: 0×IC₅₀, ¼×IC₅₀, ½×IC₅₀, 1×IC₅₀, 2×IC₅₀, 4×IC₅₀. In addition, the substrate concentration for DHO and decylubichinon were varied ¼×K_{M}, ½×K_{M}, 1×K_{M}, 2×K_{M}, 4×K_{M} in further dilution series with separate measurement of DHO and decylubiquinone. Each data point was recorded in duplicate.

The Kᵢ values for examples of the present invention were in the range of the non-deuterated matched pair (Example C26 from WO2003/006425):

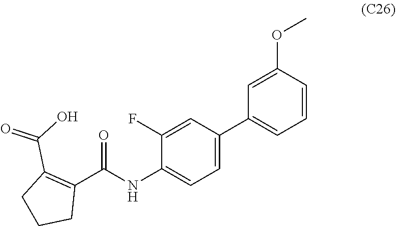 [SMILES: COc1cccc(-c2ccc(NC(=O)C3=C(C(=O)O)CCC3)c(F)c2)c1]

| | Kᵢ (DHO) | Kᵢ (decylubichinon) |
|---|---|---|
| Example # | [nM] | [nM] |
| C26 | 592 | 245 |
| 1 | 521 | 234 |
| 2 | 524 | 273 |
| 4 | 3.7 | 1.2 |
| 5 | 1820 | 839 |
| 6 | 570 | 257 |

| Example # | IC₅₀ range |
|---|---|
| C26 | ++ |
| 1/2 | +++ |
| 1/3 | +++ |
| 1/4 | ++ |
| 1/6 | +++ |
| 1/7 | ++ |
| 1/8 | 0 |
| 1/9 | ++ |
| 1/10 | ++ |
| 1/11 | 0 |
| 2/1 | +++ |
| 2/2 | +++ |
| 3 | +++ |
| 8 | + |
| 9 | 0 |
| 10 | +++ |
| 11 | ++ |
| 12 | + |
| 12/1 | +++ |
| 12/3 | 0 |
| 12/4 | + |

IC₅₀ ranges for the human DHODH assay as described herein: +++: <100 nM; ++: 100 nM to <1 μM; +: 1 μM to <10 μM; 0: ≥10 μM.

As shown above, the DHODH inhibition of deuterated analogs (i.e. 1, 2 and 6) compared to the non-deuterated matched pair (Example C26 from WO2003/006425) is not affected. Same applies for Example 4, for which the non-deuterated matched pair has a reported IC₅₀ from 7 nM (Bioorg. Med. Chem. Lett. 2005; 15:4854).

### Example 201: Microsomal Stability

Example 1 and 2 and the non-deuterated matched pair (Example C26 from WO2003/006425) were incubated using three different batches of pooled male rat liver microsomes (RLM) and human liver microsomes (HLM), respectively, for a period of 60 min. The conversion to the metabolite was monitored by HPLC-MS/MS. Verapamil served as positive control. The intrinsic clearance was calculated from the measured remaining compound values (in duplicate) at 0, 10, 30 and 60 minutes. The data points for 60 minutes are as follows:

| | | | | human liver microsomes batch | | |
|---|---|---|---|---|---|---|
| | | | | 38296 | 1210270 | 1210079 |
| Clᵢₙₜ | rat liver microsomes batch | | | mixed gender | male 10- | female 10- |
| (μl/min/mg | 4085002 | 9078001 | 1910100 | 150-donor-pool | donor-pool | donor-pool |
| protein) | (Corning) | (Corning) | (Xenotech) | (Corning) | (Xenotech) | (Xenotech) |
| Example C26 | 8.48 | 9.81 | 11.23 | 2.27 | 12.61 | 6.58 |
| Example 1 | 13.45 | 8.43 | 9.75 | 0.96 | 7.69 | 5.02 |
| Example 2 | 3.17 | 5.48 | 7.77 | 0.74 | 7.67 | 5.80 |
| Verapamil | 200.4 | 222.9 | 238.2 | 173.5 | 158.8 | 124.0 |

As exemplified with Example 1 and 2, by deuteration the intrinsic clearance in compounds of the present invention can be reduced in rat and human microsomes compared to the non-deuterated matched pair. A reduced intrinsic clearance is beneficial, since it prolonged the residence time of the drug in the body.

Example 1 and 2 and the non-deuterated matched pair (Example C26 from WO2003/006425) were incubated using three same different batches of rat (RLM) and human liver microsomes (HLM) for a period of 60 min (in duplicate i.e. 1^{st} and 2^{nd} measurement). The conversion from parent to de-methylated metabolite was monitored and quantified by HPLC-MS/MS (peak areas of mass peak) to yield the percentage of de-Me metabolite related to the initial parent (% of initial parent). With this data, the average with standard deviation (SD) was calculated.

| | Species | | Parent | de-Me metabolite | % of | Mean % | |
|---|---|---|---|---|---|---|---|
| | RLM | | (peak area) | (peak area) | initial | of initial | |
| Test Item | batch | | 0 min | 60 min | parent | parent | SD |
| Example | 4085002 | 1^{st} | 70407854 | 6464897 | 9.18 | 9.6 | 2.1 |
| C26 | | 2^{nd} | 66458354 | 5565295 | 8.37 | | |
| | 9078001 | 1^{st} | 76921121 | 5826014 | 7.57 | | |
| | | 2^{nd} | 75265781 | 5834629 | 7.75 | | |
| | 1910100 | 1^{st} | 77652247 | 9489759 | 12.22 | | |
| | | 2^{nd} | 73068386 | 9306164 | 12.74 | | |
| Example | 4085002 | 1^{st} | 68383402 | 3802248 | 5.56 | 6.2 | 2.5 |
| 1 | | 2^{nd} | 103585969 | 3438750 | 3.32 | | |
| | 9078001 | 1^{st} | 74865289 | 3583020 | 4.79 | | |
| | | 2^{nd} | 84814771 | 3520018 | 4.15 | | |
| | 1910100 | 1^{st} | 71968334 | 7056012 | 9.80 | | |
| | | 2^{nd} | 73004097 | 6959900 | 9.53 | | |
| Example | 4085002 | 1^{st} | 67670601 | 3329296 | 4.92 | 6.2 | 1.8 |
| 2 | | 2^{nd} | 60106721 | 3196275 | 5.32 | | |
| | 9078001 | 1^{st} | 65611921 | 3100045 | 4.72 | | |
| | | 2^{nd} | 64106098 | 3214860 | 5.01 | | |
| | 1910100 | 1^{st} | 74941907 | 6492165 | 8.66 | | |
| | | 2^{nd} | 72219324 | 6353644 | 8.80 | | |

As exemplified with Example 1 and 2, by selective deuteration the cleavage of the methoxy group to form the hydroxy metabolite can be reduced in rat microsomes compared to the non-deuterated matched pair (Example C26).

| | Species | | Parent | de-Me metabolite | % of | Mean % | |
|---|---|---|---|---|---|---|---|
| | HLM | | (peak area) | (peak area) | initial | of initial | |
| Test Item | batch | | 0 min | 60 min | parent | parent | SD |
| Example | 38296 | 1^{st} | 71669595 | 5684466 | 7.93 | 6.3 | 1.3 |
| C26 | | 2^{nd} | 72276431 | 5453339 | 7.55 | | |
| | 1210270 | 1^{st} | 63777868 | 4351799 | 6.82 | | |
| | | 2^{nd} | 68505784 | 4047804 | 5.91 | | |
| | 1210079 | 1^{st} | 62073486 | 2823678 | 4.55 | | |
| | | 2^{nd} | 58533090 | 2831883 | 4.84 | | |
| Example | 38296 | 1^{st} | 70932995 | 3066405 | 4.32 | 3.6 | 0.7 |
| 1 | | 2^{nd} | 71680594 | 2853522 | 3.98 | | |
| | 1210270 | 1^{st} | 60462120 | 2365635 | 3.91 | | |
| | | 2^{nd} | 55159734 | 2301305 | 4.17 | | |
| | 1210079 | 1^{st} | 54904219 | 1444490 | 2.63 | | |
| | | 2^{nd} | 58389962 | 1583025 | 2.71 | | |
| Example | 38296 | 1^{st} | 70019222 | 2766098 | 3.95 | 3.2 | 0.6 |
| 2 | | 2^{nd} | 70286951 | 2628411 | 3.74 | | |
| | 1210270 | 1^{st} | 56288300 | 1908629 | 3.39 | | |
| | | 2^{nd} | 56222644 | 1965386 | 3.50 | | |
| | 1210079 | 1^{st} | 57353703 | 1316183 | 2.29 | | |
| | | 2^{nd} | 55909814 | 1331182 | 2.38 | | |

As exemplified with Example 1 and 2, by selective deuteration the cleavage of the methoxy group to form the hydroxy metabolite can be reduced also in human microsomes compared to the non-deuterated matched pair (Example C26).

### Example 202: Rat Pharmacokinetics

The pharmacokinetics of the deuterated compounds of the present invention was evaluated in 3 male and 3 female rats (strain Han Wistar, 8 week old) after oral or intravenous cassette dosing to assess the oral bioavailability. Rats are provided with a catheter in the jugular vein (2-3 days prior to blood sampling). At each designated time point (0, 1, 2, 4, 8 and 24 h after dosing), 100 μL blood were collected into Li-heparin tubes, stored on ice until centrifugation (10 minutes at 3000 g, 4° C.) and plasma was prepared within 45 min after collection, frozen at −20° C. and stored at this temperature until processed for LC-MS analysis. The obtained data is as follows:

| Gender | male | | female | |
|---|---|---|---|---|
| Test item | Comparative example C26 | | | |
| Dose route | po | iv | po | iv |
| Vehicle | PEG 300 | | | |
| Dosage (mg/kg) | 5 | 1 | 5 | 1 |
| Volume (ml/kg) | 5 | 2 | 5 | 2 |
| Cmax (ng/ml) | 14936.0 | — | 21334.3 | — |
| C0 (ng/ml) | — | 5787 | — | 8130 |
| tmax (h) | 2.0 | — | 2.0 | — |
| Cz (ng/ml) | 1540 | 421 | 3620 | 939 |
| tz (h) | 24 | 24 | 24 | 24 |
| t1/2z (h) | 6.4 | 7.3 | 9.1 | 9.7 |
| AUC0-tz(ng*h/ml) | 200498 | 43239 | 253138 | 68625 |
| AUC0-∞(ng*h/ml) | 205788 | 47852 | 297424 | 82937 |
| Vz/f (ml/kg) | 7 | 9 | 16 | 16 |
| CL/f (ml/(h*kg)) | 223 | 225 | 222 | 168 |
| % AUCextra | 25 | 22 | 17 | 12 |
| Bioavailability (%) | 92.7 | — | 73.8 | — |
| Gender | male | | female | |
| Test item | Example 1 | | | |
| Dose route | po | iv | po | iv |
| Vehicle | PEG 300 | | | |
| Dosage (mg/kg) | 5 | 1 | 5 | 1 |
| Volume (ml/kg) | 5 | 2 | 5 | 2 |
| Cmax (ng/ml) | 14588 | — | 19697 | — |
| C0 (ng/ml) | — | 5541 | — | 7012 |
| tmax (h) | 2.0 | — | 2.0 | — |
| Cz (ng/ml) | 1525 | 437 | 3536 | 943 |
| tz (h) | 24 | 24 | 24 | 24 |
| t1/2z (h) | 6.6 | 7.9 | 9.1 | 9.9 |
| AUC0-tz(ng*h/ml) | 183255 | 42623 | 249133 | 66205 |
| AUC0-∞(ng*h/ml) | 209643 | 47879 | 291424 | 80697 |
| Vz/f (ml/kg) | 8 | 10 | 16 | 17 |
| CL/f (ml/(h*kg)) | 229 | 242 | 226 | 176 |
| % AUCextra | 24 | 22 | 17 | 13 |
| Bioavailability (%) | 86.0 | — | 75.3 | — |
| Gender | male | | female | |
| Test item | Example 2 | | | |
| Dose route | po | iv | po | iv |
| Vehicle | PEG 300 | | | |
| Dosage (mg/kg) | 5 | 1 | 5 | 1 |
| Volume (ml/kg) | 5 | 2 | 5 | 2 |
| Cmax (ng/ml) | 16050 | — | 21276 | — |
| C0 (ng/ml) | — | 5309 | — | 7983 |
| tmax (h) | 2.0 | — | 2.0 | — |
| Cz (ng/ml) | 1657 | 422 | 3892 | 915 |
| tz (h) | 24 | 24 | 24 | 24 |
| t1/2z (h) | 6.6 | 7.6 | 9.3 | 10.0 |
| AUC0-tz(ng*h/ml) | 201953 | 40393 | 268689 | 64698 |
| AUC0-∞(ng*h/ml) | 232828 | 45215 | 319543 | 79125 |
| Vz/f (ml/kg) | 8 | 10 | 17 | 17 |
| CL/f (ml/(h*kg)) | 206 | 247 | 210 | 181 |
| % AUCextra | 22 | 23 | 16 | 13 |
| Bioavailability (%) | 100.0 | — | 83.1 | — |

The non-deuterated compound vidofludimus (Comparative example C26) itself has already a quite good bioavailability. By selective deuteration (Example 2), this bioavailability can be further improved, which can be attributed to the diminished metabolism.

### Example 203: Mouse Pharmacokinetics

The pharmacokinetics of the compounds of the present invention was evaluated in 3 male and 3 female mice (CC7BL/6J, 8 week old) after oral cassette dosing. Dose was 5 mg/kg, application volume was 5 mL/kg and vehicle was 5% Solutol, 95% NaCl solution (at 0.9% saline concentration). At each designated time point (0, 0.25, 0.5, 1, 2, 4, 8 h after dosing), 20 μL whole blood were collected from the tail vein into Li-heparin tubes, frozen on dry ice within 1-2 minutes of sampling and stored at −20° C. until processed for LC-MS analysis. The obtained data is as follows:

| Example # | Cₘₐₓ | t_{1/2} | AUC |
|---|---|---|---|
| Example C26 | ♂ 789 ng/mL | ♂ 1.7 h | ♂ 1620 ng*h/mL |
| | ♀ 745 ng/mL | ♀ 1.7 h | ♀ 1970 ng*h/mL |
| Example 1 | ♂ 4110 ng/mL | ♂ 1.7 h | ♂ 5110 ng*h/mL |
| | ♀ 3440 ng/mL | ♀ 1.6 h | ♀ 5740 ng*h/mL |

Abbreviations: ♂ = male, ♀ = female, Cₘₐₓ = peak plasma concentration, t_{1/2} = elimination half-life, AUC = area under the curve (integral of the concentration-time curve from 0 to 8 h)

Again, the non-deuterated Comparative example C26 has lower Cₘₐₓ and AUC values, which can be dramatically improved by selective deuteration (Example 1).

### Example 204: Antiviral Activity on SARS-CoV-2

The assay for viral replication (YFP) and the cell viability assay has been described in general in Pathogens 2021; 10:1076 and applied to compounds of the present invention furnished the following results:

| Example # | EC₅₀ range | CC₅₀ range |
|---|---|---|
| 1 | +++ | >100 |
| 1/2 | +++ | >100 |
| 1/3 | +++ | >100 |
| 1/4 | +++ | >100 |
| 1/6 | +++ | >100 |
| 1/7 | +++ | >100 |
| 1/8 | + | >100 |
| 1/9 | + | >100 |
| 1/10 | ++ | >100 |
| 3 | +++ | >100 |
| 8 | + | >100 |
| 9 | ++ | >100 |
| 10 | ++ | >100 |
| 11 | 0 | >100 |
| 12 | +++ | 76 |
| 12/1 | +++ | >100 |
| 12/3 | + | >100 |
| 12/4 | ++ | >100 |

EC₅₀ ranges for the SARS-CoV-2 assay as described herein: +++: < 10 μM; ++: 10 μM to <25 μM; +: 25 μM to <50 μM; 0: ≥50 μM.

### Example 205: Synergistic Antiviral Activity on SARS-CoV-2 with a Nucleoside Analogue

The synergistic potential of Example 9 together with the nucleoside analogue EIDD-1931 (CAS: 3258-02-4) was assessed.

The method of combinatorial drug assessment by a viral replication inhibition assay has been published in Pathogens 2021; 10:1076. Caco-2 cells were cultivated in 96-well plates at 25000 cells/well, infected with SARS-CoV-2 d6-YFP at an MOI of 0.003 and treated with Example 9, EIDD-1931 or a combination of the drugs, starting at the respective 4×EC₅₀ concentrations of the single compounds. Viral replication was determined as 30 h post infection (p.i.) by quantitative fluorescence detection of virus-driven YFP expression in the fixed cells. Inhibitory profiles of viral replication measured through virus-encoded YFP reporter expression are presented in a bar chart of quadruplicate determinations (mean±SD). The combinatorial drug assessment was calculated by using the CompuSyn algorithm as described in Int. J. Mol. Sci. 2021; 22:575.

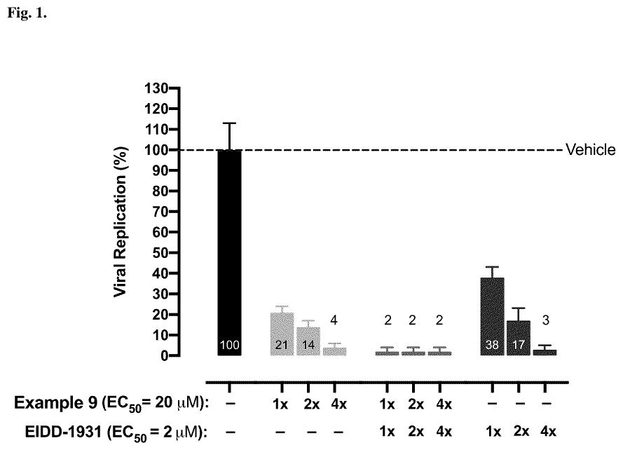

A representative experiment is shown in FIG. 1. Compound of Example 9 shows synergistic antiviral effects on SARS-CoV-2 when combined with nucleoside analogue EIDD-1931 (CAS: 3258-02-4).

1. A compound according to Formula (I): 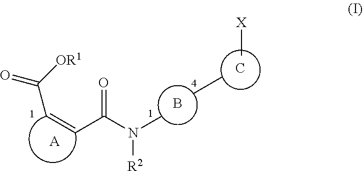
or an enantiomer, diastereomer, tautomer, prodrug, solvate, or pharmaceutically acceptable salt thereof, wherein
A is selected from a 5-membered heteroaryl, cyclopentenyl and heterocyclopentenyl, having one or more hydrogen atoms optionally replaced by deuterium,
said A is unsubstituted or substituted with 1 to 5 substituents independently selected from the group consisting of halogen, CN, NO₂, oxo, OH, C₁₋₄-alkyl, O—C₁₋₄-alkyl, fluoro-C₁₋₄-alkyl and O-fluoro-C₁₋₄-alkyl, CO₂H and SO₃H, having one or more hydrogen atoms in alkyl optionally replaced by deuterium;
B is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S,
wherein cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR²⁷, C₀₋₆-alkylene-(3- to 6-membered cycloalkyl), C₀₋₆-alkylene-(3- to 6-membered heterocycloalkyl), C₀₋₆-alkylene-S(═O)ₙ(═NR²⁹)ₘR²⁷, C₀₋₆-alkylene-NR²⁷S(═O)ₓ(═NR²⁹)_{y}R²⁷, C₀₋₆-alkylene-S(═O)ₓ(═NR²⁹)_{y}NR²⁷R²⁸, C₀₋₆-alkylene-NR²⁷ S(═O)ₓ(═NR²⁹)_{y}NR²⁷R²⁸, C₀₋₆-alkylene-CO₂R²⁷, C₀₋₆-alkylene-O—COR²⁷, C₀₋₆-alkylene-CONR²⁷R²⁸, C₀₋₆-alkylene-NR²⁷—COR²⁷, C₀₋₆-alkylene-NR²⁷—CONR²⁷R²⁸, C-₀₋₆-alkylene-O—CONR²⁷R²⁸, C₀₋₆-alkylene-NR²⁷—CO₂R²⁷, C₀₋₆-alkylene-NR²⁷R²⁸,
wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl;
and wherein optionally two adjacent substituents in the aryl or heteroaryl moiety form a 5- to 8-membered partially unsaturated cycle optionally containing 1 to 3 heteroatoms independently selected from O, S or N, wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
and wherein the residue —NR² on ring B is in a 1,4-orientation with respect to ring C, ring B or its substituents having one or more hydrogen atoms optionally replaced by deuterium;
C is selected from the group consisting of 5- to 10-membered cycloalkyl, 4- to 10-membered heterocycloalkyl containing 1 to 4 heteroatoms independently selected from N, O and S, 6- or 10-membered aryl and 5- to 10-membered heteroaryl containing 1 to 6 heteroatoms independently selected from N, O and S,
wherein cycloalkyl, heterocycloalkyl, aryl and heteroaryl are unsubstituted or substituted with 1 to 4 substituents independently selected from the group consisting of halogen, —CN, —NO₂, oxo, C₁₋₄-alkyl, C₀₋₆-alkylene-OR³¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR³³)ₘR³¹, C₀₋₆-alkylene-NR³¹S(═O)ₓ(═NR³³)_{y}R³¹, C₀₋₆-alkylene-S(═O)ₓ(═NR³³)_{y}NR³¹R³², C₀₋₆-alkylene-NR³¹ S(═O)ₓ(═NR³³)_{y}NR³¹R³², C₀₋₆-alkylene-CO₂R³¹, C₀₋₆-alkylene-O—COR³¹, C₀₋₆-alkylene-CONR³¹R³², C₀₋₆-alkylene-NR³¹—COR³¹, C₀₋₆-alkylene-NR³¹—CONR³¹R³², C₀₋₆-alkylene-O—CONR³¹R³², C₀₋₆-alkylene-NR³¹—CO₂R³¹, C₀₋₆-alkylene-NR³¹R³²,
wherein alkyl, alkylene, 3- to 6-membered cycloalkyl and 3- to 6-membered heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl;
and wherein optionally two adjacent substituents in the aryl or heteroaryl moiety form a 5- to 8-membered partially unsaturated cycle optionally containing 1 to 3 heteroatoms independently selected from O, S or N,
wherein this additional cycle is optionally substituted with 1 to 4 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
ring C or its substituents having one or more hydrogen atoms optionally replaced by deuterium;
X is selected from H, D, halogen, —CN, —NO₂, C₁₋₆-alkyl, —O—C₁₋₆-alkyl, O-halo-C₁₋₆-alkyl, C₀₋₆-alkylene-OR⁴¹, C₀₋₆-alkylene-(3- to 6-membered cycloalkyl), C₀₋₆-alkylene-(3- to 6-membered heterocycloalkyl), C₀₋₆-alkylene-S(═O)ₙ(═NR⁴³)ₘR⁴¹, C₀₋₆-alkylene-NR⁴¹S(═O)ₓ(═NR⁴³)_{y}R⁴¹, C₀₋₆-alkylene-S(═O)ₓ(═NR⁴³)_{y}NR⁴¹R⁴², C₀₋₆-alkylene-NR⁴¹S(═O)ₓ(═NR⁴³)_{y}NR⁴¹R⁴², C₀₋₆-alkylene-CO₂R⁴¹, C₀₋₆-alkylene-O—COR⁴¹, C₀₋₆-alkylene-CONR⁴¹R⁴², C₀₋₆-alkylene-NR⁴¹—COR⁴¹, C₀₋₆-alkylene-NR⁴¹—CONR⁴¹R⁴², C₀₋₆-alkylene-O—CONR⁴¹R⁴², C₀₋₆-alkylene-NR⁴¹—CO₂R⁴¹, C₀₋₆-alkylene-NR⁴¹R⁴², wherein heterocycloalkyl comprises 1, 2, 3 or 4 heteroatoms independently selected from N, O, or S,
wherein alkyl, alkylene, cycloalkyl and heterocycloalkyl is unsubstituted or substituted with 1 to 6 substituents independently selected from halogen, —CN, oxo, —OH, C₁₋₄-alkyl, halo-C₁₋₄-alkyl, —O—C₁₋₄-alkyl and —O-halo-C₁₋₄-alkyl,
X or its substituents having one or more hydrogen atoms optionally replaced by deuterium;
R¹ is selected from H and D;
R² is selected from H and C₁₋₆-alkyl,
wherein alkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R² or its substituents having one or more hydrogen atoms optionally replaced by deuterium;
R²⁷, R²⁸, R³¹, R³², R⁴¹, R⁴² are independently selected from H, C₁₋₆-alkyl, 3- to 6-membered cycloalkyl or 3- to 6-membered heterocycloalkyl,
wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R²⁷ and/or R²⁸ and/or R³¹ and/or R³² and/or R⁴¹ and/or R⁴² or its substituents, respectively, having one or more hydrogen atoms optionally replaced by deuterium;
or R²⁷ and R²⁸, R³¹ and R³², R⁴¹ and R⁴², respectively, when taken together with the nitrogen to which they are attached complete a 3- to 6-membered cycle containing carbon atoms and optionally containing 1 or 2 heteroatoms selected from O, S or N; and
wherein this cycle is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl,
R²⁷ and/or R²⁸ and/or R³¹ and/or R³² and/or R⁴¹ and/or R⁴² or its substituents, respectively, having one or more hydrogen atoms optionally replaced by deuterium;
R²⁹, R³³, R⁴³ are independently selected from H, —CN, —NO₂, C₁₋₆-alkyl, —CO—O—C₁₋₆-alkyl, 3- to 6-membered cycloalkyl or 3- to 6-membered heterocycloalkyl,
wherein alkyl, cycloalkyl or heterocycloalkyl is unsubstituted or substituted with 1 to 3 substituents independently selected from halogen, —CN, C₁₋₄-alkyl, halo-C₁₋₄-alkyl, 3- to 6-membered cycloalkyl, halo-(3- to 6-membered cycloalkyl), 3- to 6-membered heterocycloalkyl, halo-(3- to 6-membered heterocycloalkyl), —OH, oxo, —O—C₁₋₄-alkyl and —O-halo-C₁₋₄-alkyl, wherein heterocycloalkyl comprises 1, 2, 3 or 4 heteroatoms independently selected from N, O, or S,
R²⁹ and/or R³³ and/or R⁴³ or its substituents, respectively, having one or more hydrogen atoms optionally replaced by deuterium;
n, m, x, y are independently selected from 0 to 2;
with the proviso that the sum of integer m and n for the residue linked to the same sulfur atom is independently selected from 0 to 2;
with the proviso that the sum of integer x and y for the residue linked to the same sulfur atom is independently selected from 1 or 2;
provided that at least one hydrogen in A, B, C, R², R²⁷, R²⁸, R²⁹, R³¹, R³², R³³, R⁴¹, R⁴², R⁴³ and/or X is replaced by deuterium;
provided, that the level of deuterium incorporation at each substituent designated as deuterium is at least 52.5%.

2. A compound of Formula (I) according to claim 1, or a solvate or pharmaceutically acceptable salt thereof, wherein
R¹ is H and R² is H.

3. A compound of Formula (I) according to claim 1, wherein 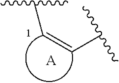
is selected from

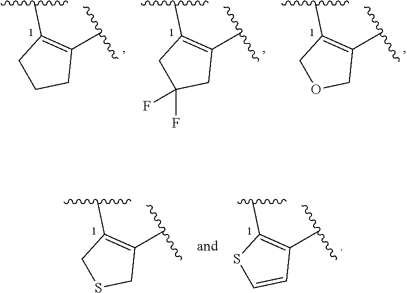 [SMILES: *C1=C(*)CCC1 ; *C1=C(*)CC(F)(F)C1 ; *C1=C(*)COC1 ; *C1=C(*)CSC1 ; *c1ccsc1*]

4. A compound of Formula (I) according to claim 1, wherein
—NR²B is selected from

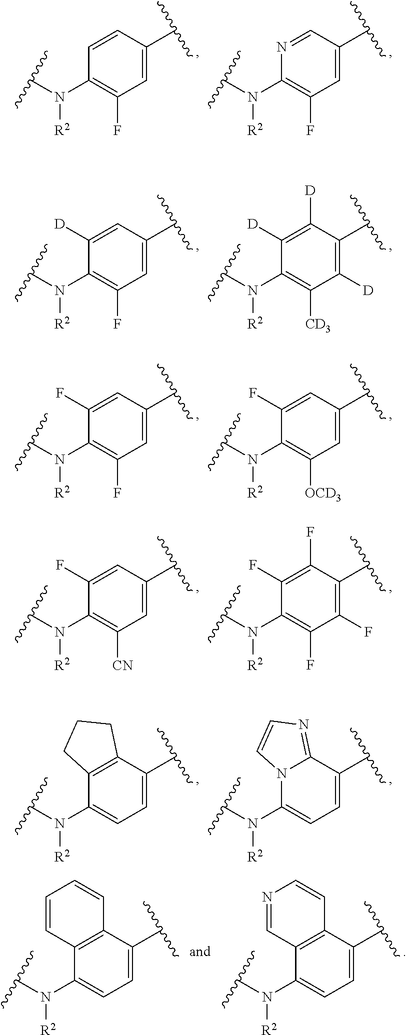

5. A compound of Formula (I) according to claim 1, wherein
C is phenyl, pyridyl or thiazolyl,
wherein phenyl, pyridyl or thiazolyl is unsubstituted or substituted with 1 to 4 substituents independently selected from the group consisting of D and F; and
X is selected from D, F, Cl, —CN, OH, C₁₋₄-alkyl, O—C₁₋₄-alkyl, fluoro-C₁₋₄-alkyl, O-fluoro-C₁₋₄-alkyl, having one or more hydrogen atoms optionally replaced by deuterium.

6. A compound of Formula (I) according to claim 1, wherein 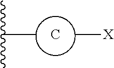
is selected from

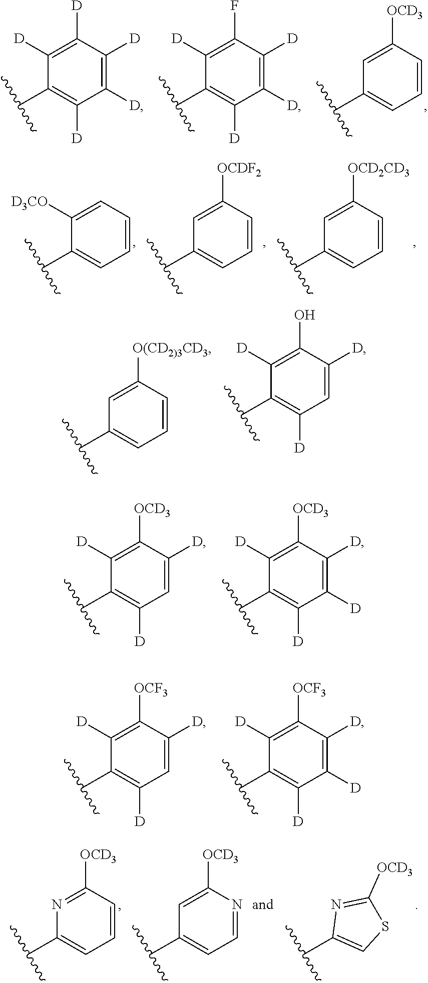

7. A compound of Formula (I) according to claim 1, wherein 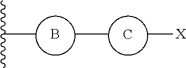
is selected from

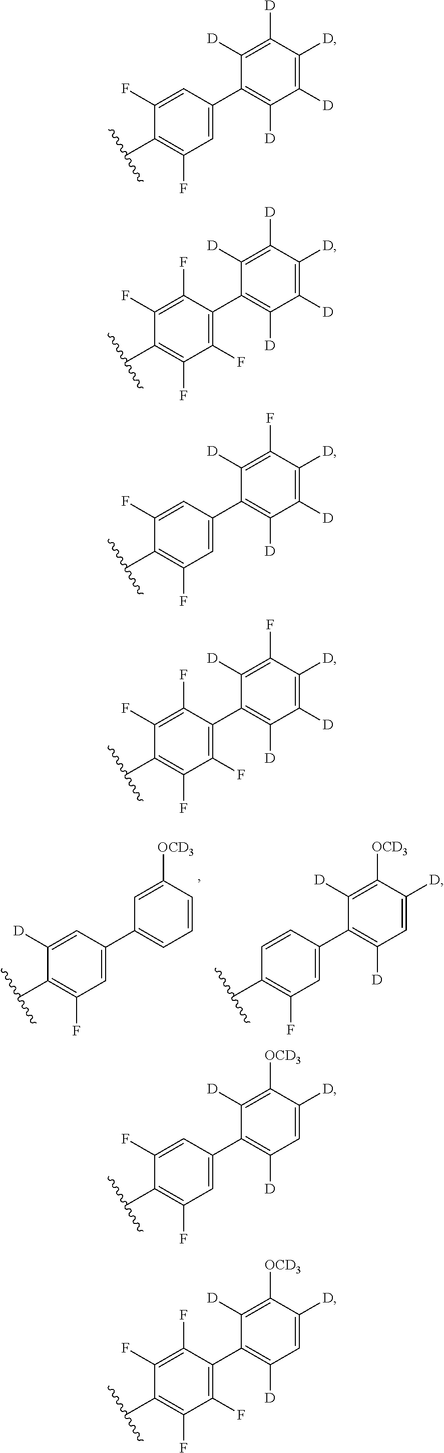

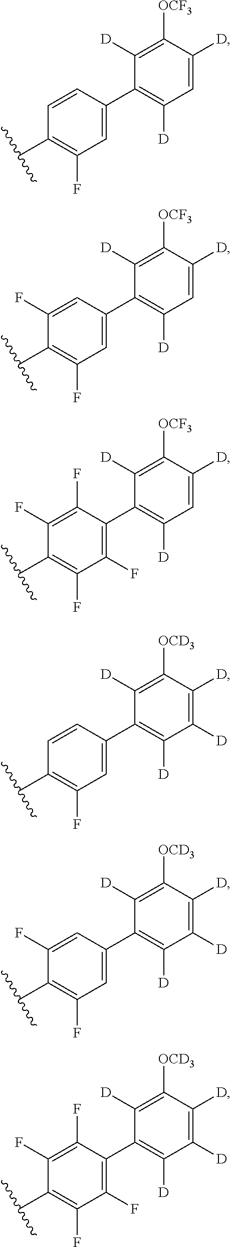

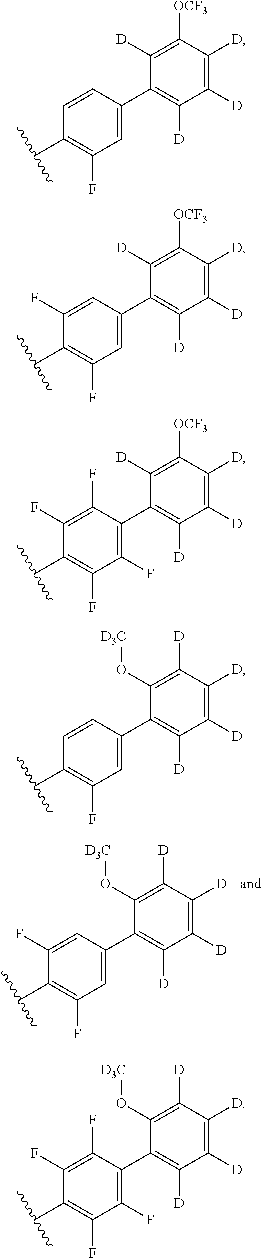

8. A compound of Formula (I) according to claim 1, wherein
R¹ is H and R¹ is H; 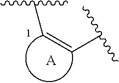
is selected from 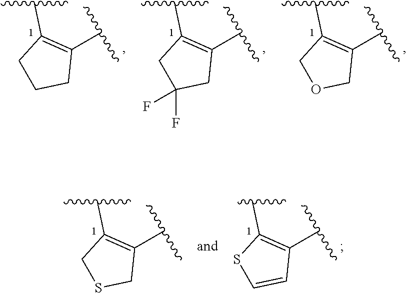 [SMILES: *C1=C(*)CCC1 ; *C1=C(*)CC(F)(F)C1 ; *C1=C(*)COC1 ; *C1=C(*)CSC1 ; *c1ccsc1*]
—NR²B is selected from 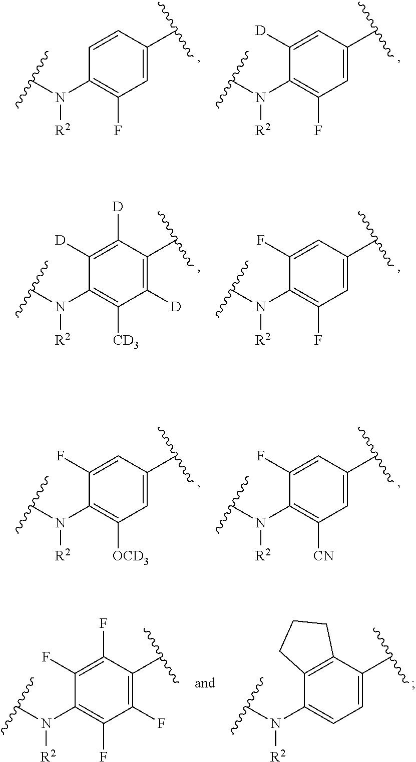
and 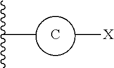
is selected from

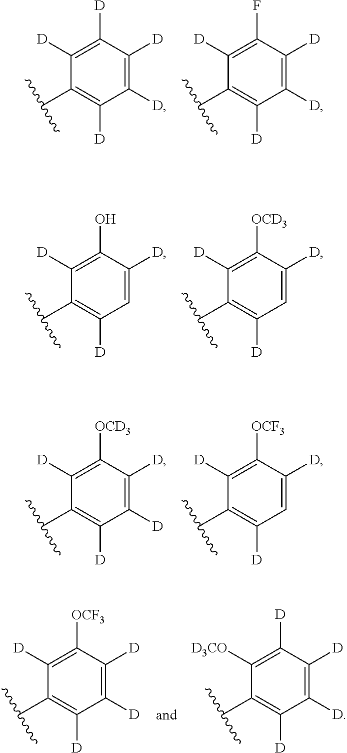

9. A compound of Formula (I) according to claim 1, which is selected from 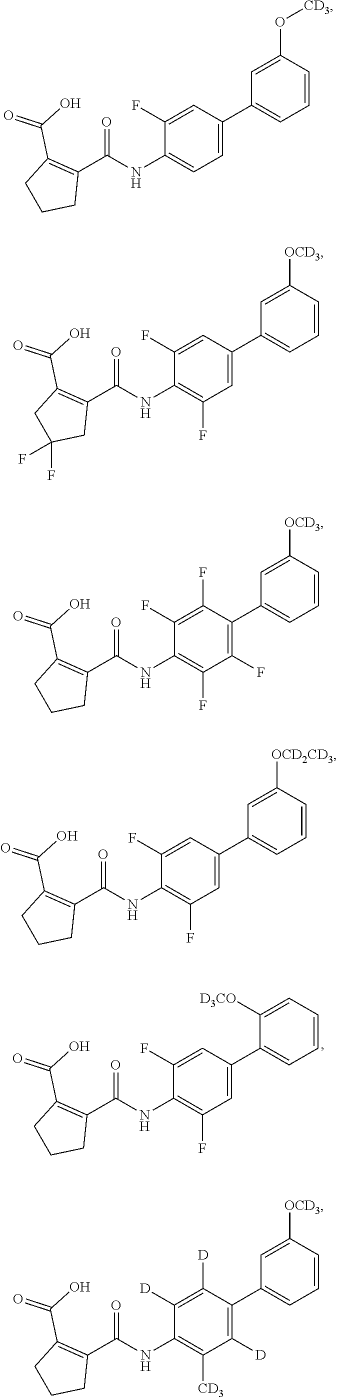 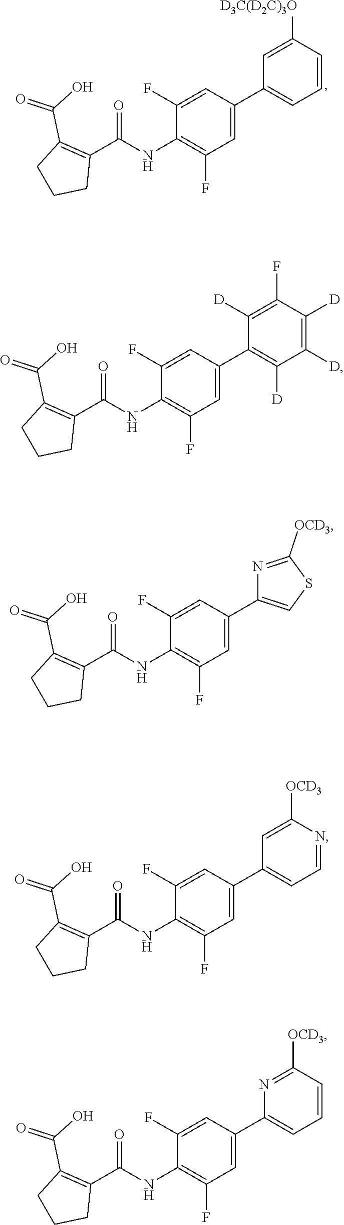 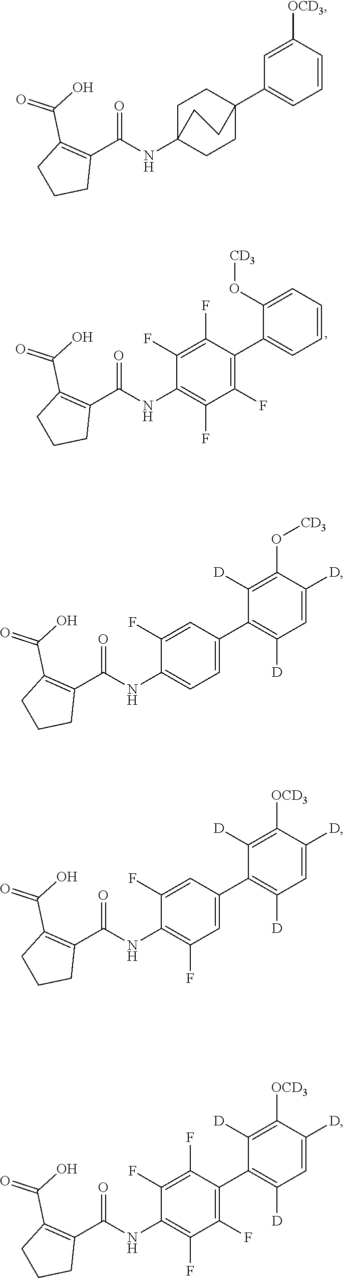 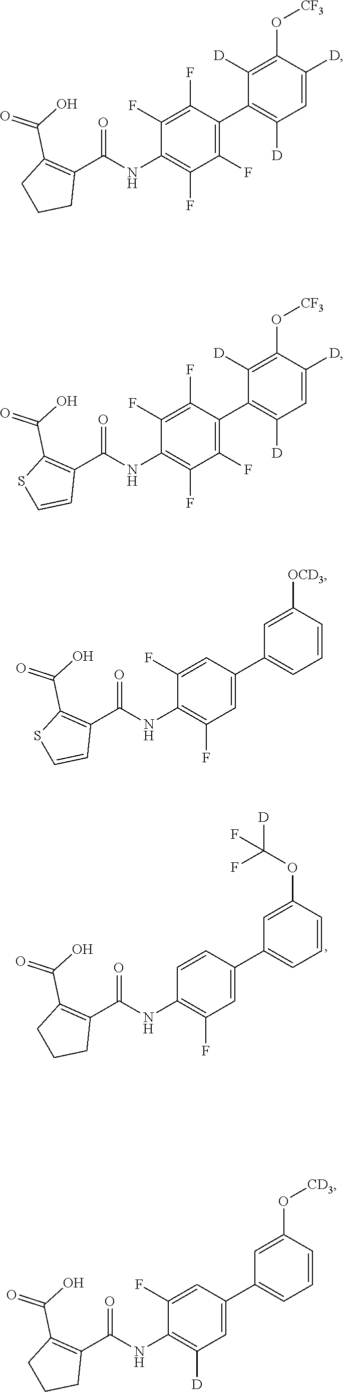 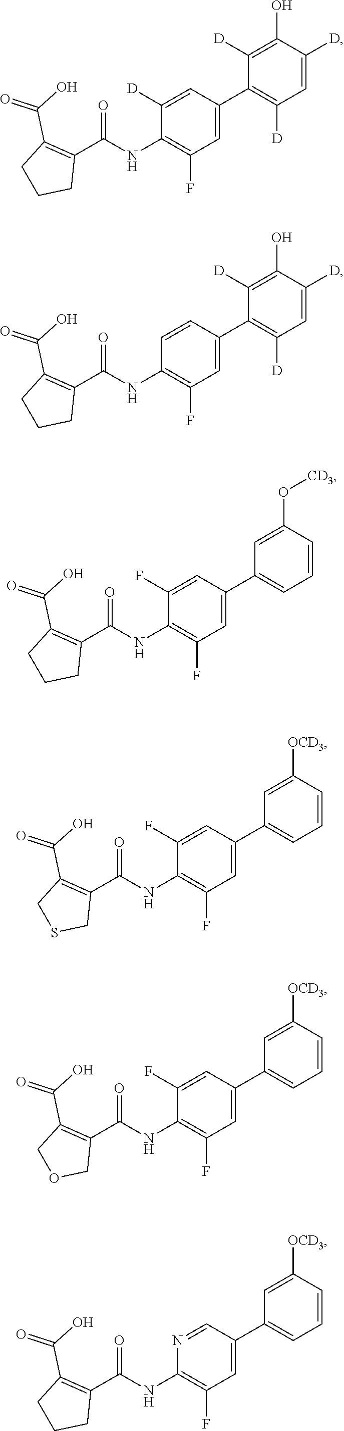 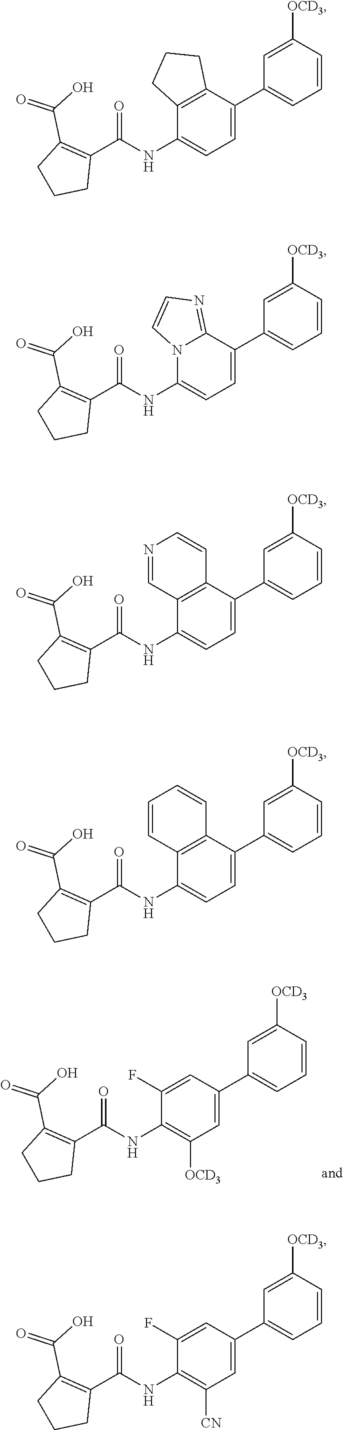
or a solvate or pharmaceutically acceptable salt thereof.

10. A method of treating a disease in a subject in need thereof, comprising administering to the subject a compound according to claim 1.

11. A method of preventing a disease in a subject in need thereof, comprising administering to the subject a compound according to claim 1.

12. The method according to claim 10, wherein the disease is selected from rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation, and arthropathy.

13. The method according to claim 10, wherein the disease is selected from graft versus host and host versus graft reactions, rheumatoid arthritis, multiple sclerosis, amyotrophic lateral sclerosis, lupus erythematosus, inflammatory bowel disease, cancer, COVID-19, influenza, ulcerative colitis, Crohn's disease, primary sclerosing cholangitis, and psoriasis.

14. A pharmaceutical composition comprising a compound according to claim 1 and a pharmaceutically acceptable carrier or excipient.

15. The pharmaceutical composition of claim 14, further comprising one or more therapeutic agents selected from antiviral agents, anti-inflammatory agents, immunosuppressive and/or immunomodulatory agents, steroids, non-steroidal anti-inflammatory agents, antihistamines, analgesics, and suitable mixtures thereof.

16. A compound of Formula (I), according to claim 1, which is 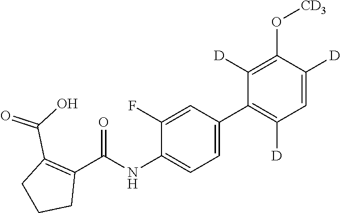 [SMILES: [2H]c1cc([2H])c(-c2ccc(NC(=O)C3=C(C(=O)O)CCC3)c(F)c2)c([2H])c1OC([2H])([2H])[2H]]
or a solvate or pharmaceutically acceptable salt thereof.

17. The method according to claim 11, wherein the disease is selected from rheumatism, acute immunological disorders, autoimmune diseases, diseases caused by malignant cell proliferation, inflammatory diseases, diseases that are caused by protozoal infestations in humans and animals, diseases that are caused by viral infections and Pneumocystis carinii, fibrosis, uveitis, rhinitis, asthma, transplantation, and arthropathy.

18. The method according to claim 11, wherein the disease is selected from graft versus host and host versus graft reactions, rheumatoid arthritis, multiple sclerosis, amyotrophic lateral sclerosis, lupus erythematosus, inflammatory bowel disease, cancer, COVID-19, influenza, ulcerative colitis, Crohn's disease, primary sclerosing cholangitis, and psoriasis.
